(19) United States
(12) Patent Application Publication
(10) Pub. No.: US 2023/0201274 A1
(43) Pub. Date: Jun. 29, 2023

(54) SHELF- STABLE AMMONIA OXIDIZING MICROORGANISM PREPARATIONS

(71) Applicant: AOBIOME LLC, Cambridge, MA (US)

(72) Inventor: Carson Alexis Goddard, Weston, MA (US)

(21) Appl. No.: 17/926,094

(22) PCT Filed: May 21, 2021
(86) PCT No.: PCT/US2021/033754

§ 371 Date: Nov. 17, 2022

Related U.S. Application Data
(60) Provisional application No. 63/028,364, filed on May 21, 2020.

(51) Int. Cl. A61K 35/74; A61K 8/99; A61K 9/06; A61K 35/17

(52) U.S. Cl. CPC A61K 35/74; A61K 8/99; A61K 9/06; A61K 35/17; A61K 2800/10; A61K 2800/87

(57) ABSTRACT

Methods of distributing a preparation including providing the preparation comprising ammonia oxidizing microorganisms (AOM), wherein, during distribution, the preparation is exposed to a temperature greater than about 4° C. are disclosed. Methods of treating a subject comprising administering the preparation wherein less than 70% of the AOM are viable are also disclosed. Methods of treating a subject comprising administering the preparation at room temperature are also disclosed. Methods of treating Autosomal dominant hyper- IgE syndrome (AD-HIES) with preparations comprising AOM are disclosed. Methods of treating immunodysregula-tion polyendocrinopathy enteropathy-X-linked (IPEX) with preparations comprising AOM are disclosed. Methods of producing shelf-stable products from the preparation are also disclosed. Shelf- stable preparations of AOM are also disclosed.

### FIELD OF THE TECHNOLOGY

Aspects relate generally to the microbiome and, more specifically, to the restoration of ammonia oxidizing microorganisms in relation to the microbiome.

### BACKGROUND

Bacteria and other microorganisms are ubiquitous in the environment. The discovery of pathogenic bacteria and the germ theory of disease have had a tremendous effect on health and disease states. Microorganisms are a normal part of the environment of all living things and may be beneficial. In the gut, for example, bacteria are not pathogenic under normal conditions, and in fact improve health by rendering the normal intestinal contents less hospitable for disease causing organisms.

### SUMMARY

In accordance with one aspect, there is provided a method of distributing a preparation. The method may include providing a preparation comprising live ammonia oxidizing microorganisms (AOM). In some embodiments, during distribution, the packaged end use container is exposed to an environment having a temperature greater than about 4° C.

In accordance with another aspect, there is provided a method of treating a subject. The method may comprise administering to the subject a therapeutically effective amount of a preparation comprising ammonia oxidizing microorganisms (AOM). In some embodiments, less than about 70%, 60%, 50%, 40%, 30%, 20%, 10%, 5%, 1%, 0.5%, or 0.1% of the ammonia oxidizing microorganisms are viable.

In accordance with another aspect, there is provided a method of treating a subject. The method may comprise administering to the subject a therapeutically effective amount of a preparation comprising ammonia oxidizing microorganisms (AOM). In some embodiments, the preparation is at a temperature greater than or about room temperature e.g., between about 20° C. - 25° C., when administered.

In accordance with another aspect, there is provided a method of treating Autosomal dominant hyper-IgE syndrome (AD-HIES) in a subject. The method may comprise administering to the subject a therapeutically effective amount of a preparation comprising ammonia oxidizing microorganisms (AOM).

In accordance with another aspect, there is provided a method of treating immunodysregulation polyendocrinopathy enteropathy-X-linked (IPEX) in a subject. The method may comprise administering to the subject a therapeutically effective amount of a preparation comprising ammonia oxidizing microorganisms (AOM).

In accordance with another aspect, there is provided a method of producing a shelf-stable cosmetic, therapeutic, or consumer product. The method may comprise formulating a preparation comprising ammonia oxidizing microorganisms (AOM) into a powder, cream, ointment, or lotion. The method may comprise packaging the preparation into the cosmetic, therapeutic, or consumer product. In some embodiments, less than about 70%, 60%, 50%, 40%, 30%, 20%, 10%, 5%, 1%, 0.5%, or 0.1% of the ammonia oxidizing microorganisms in the cosmetic or therapeutic product are viable.

The method may comprise providing the preparation comprising live ammonia oxidizing microorganisms.

In some embodiments, less than about 70%, 60%, 50%, 40%, 30%, 20%, 10%, 5%, 1%, 0.5%, or 0.1% of the ammonia oxidizing microorganisms are viable.

In some embodiments, the preparation is exposed to an environment having a temperature greater than about 4° C.

In some embodiments, the preparation is packaged into a container.

In some embodiments, the preparation is packaged into a plurality of containers, e.g., at least 2, 3, 6, 8, 10, or 20 containers.

In some embodiments, the preparation is packaged into an end use container.

In some embodiments, the preparation is packaged into a plurality of separate end use containers, e.g., at least 2, 4, 6, 8, 10, 20, 50, or 100 end use containers.

The method may comprise supplying (or causing a designee to supply) the preparation or packaged end use container to a recipient.

In some embodiments, during distribution, the preparation or packaged end use container is exposed to an environment having a temperature greater than about 10° C.

In some embodiments, during distribution, the preparation or packaged end use container is exposed to an environment having a room temperature, e.g., between about 20° C. - 25° C.

In some embodiments, during distribution, the preparation or packaged end use container attains a temperature greater than about 4° C.

In some embodiments, during distribution, the preparation or packaged end use container attains a temperature greater than about 10° C.

In some embodiments, during distribution, the preparation or packaged end use container attains a temperature greater than room temperature, e.g., between about 20° C. - 25° C.

In some embodiments, during distribution, the preparation or packaged end use container is exposed to the environment for a period of time of at least about 1 hour, 6 hours, 12 hours, 18 hours, 1 day, 3 days, 5 days, 1 week, 2 weeks, 3 weeks, 4 weeks, 2 months, 3 months, 4 months, 5 months, 6 months, 1 year, or 5 years.

The environment may be a storage environment.

The environment may be a shipping environment, e.g., a mail or a commercial delivery shipping environment.

The environment may be a shipping environment, e.g., a cargo or a freight transport shipping environment.

In some embodiments, during distribution, the preparation or packaged end use container attains a temperature greater than about 4° C. for at least about 1 hour, 6 hours, 12 hours, 18 hours, 1 day, 3 days, 5 days, 1 week, 2 weeks, 3 weeks, 4 weeks, 2 months, 3 months, 4 months, 5 months, 6 months, 1 year, or 5 years.

In some embodiments, during distribution, the preparation or packaged end use container attains a temperature greater than about 10° C. for at least about 1 hour, 6 hours, 12 hours, 18 hours, 1 day, 3 days, 5 days, 1 week, 2 weeks, 3 weeks, 4 weeks, 2 months, 3 months, 4 months, 5 months, 6 months, 1 year, or 5 years.

In some embodiments, during distribution, the preparation or packaged end use container attains a temperature greater than about room temperature, e.g., between about 20° C. - 25° C., for at least about 1 hour, 6 hours, 12 hours, 18 hours, 1 day, 3 days, 5 days, 1 week, 2 weeks, 3 weeks, 4 weeks, 2 months, 3 months, 4 months, 5 months, 6 months, 1 year, or 5 years.

In some embodiments, supplying (or causing the designee to supply) the preparation or packaged end use container to the recipient may comprise making the packaged end use container available on an internet-based outlet.

In some embodiments, supplying (or causing the designee to supply) the preparation or packaged end use container to the recipient may comprise making the packaged end use container available at a non-internet-based outlet, e.g., a store.

In some embodiments, upon packaging, e.g., into an end use container, at least about 30%, 40%, 50%, 60%, 70%, 80%, 90%, 95%, 99%, 99.5%, or 99.9% of the ammonia oxidizing microorganisms are viable.

In some embodiments, upon packaging, e.g., into an end use container, at least about 30%, 40%, 50%, 60%, 70%, 80%, 90%, 95%, 99%, 99.5%, or 99.9% of the ammonia oxidizing microorganisms are active.

In some embodiments, after distribution, less than about 70%, 60%, 50%, 40%, 30%, 20%, 10%, 5%, 1%, 0.5%, or 0.1% of the ammonia oxidizing microorganisms are viable.

In some embodiments, after distribution, at least about 30%, 40%, 50%, 60%, 70%, 80%, 90%, 95%, 99%, 99.5%, or 99.9% of the ammonia oxidizing microorganisms are inactive.

In some embodiments, upon packaging, e.g., into an end use container, less than about 70%, 60%, 50%, 40%, 30%, 20%, 10%, 5%, 1%, 0.5%, or 0.1% of the ammonia oxidizing microorganisms are viable.

In some embodiments, upon packaging, e.g., into an end use container, less than about 70%, 60%, 50%, 40%, 30%, 20%, 10%, 5%, 1%, 0.5%, or 0.1% of the ammonia oxidizing microorganisms are inactive.

In some embodiments, at least about 30%, 40%, 50%, 60%, 70%, 80%, 90%, 95%, 99%, 99.5%, 99.9%, or substantially all of the ammonia oxidizing microorganisms are heat killed, e.g., prior to packaging.

In some embodiments, the container, e.g., end use container, comprises a polymer bottle, e.g., a spray, aerosol, or mist bottle.

In some embodiments, the container, e.g., end use container, comprises a squeezable container, e.g., squeeze bottle or tube.

In some embodiments, the container, e.g., end use container, is substantially free of a vacuum bag.

In some embodiments, the container, e.g., end use container, is not configured to inhibit or reduce retrograde flow.

In some embodiments, the container, e.g., end use container comprises polymer, e.g., polyethylene terephthalate (PET), high density polyethylene (HDPE), polypropylene, polycarbonate, polytetrafluoroethylene (teflon®), polyviylidene fluoride (PVDF), or a cellulosic, glass, aluminum, or cardboard.

In some embodiments, the container, e.g., end use container, is configured to allow passage of oxygen.

In some embodiments, the container, e.g., end use container, is configured to allow passage of at least about 5, 10, 20, 30, 40, 50, 60, 70, 80, 90, 95, 99, or 100 percent of transmission of ionizing radiation, e.g., with gamma rays, e.g., with x-rays, e.g., from an isotope, e.g., cobalt 60, or with ultraviolet, e.g., ultraviolet C (UVC) through the container.

The method may comprise treating a disease or disorder modulated by an activated immune cell in a subject.

In some embodiments, the activated immune cell is a T helper cell or a regulatory T cell.

In some embodiments, the activated immune cell is T helper type 1 (Th1), T helper type 2 (Th2), T helper type 17 (Th17), or regulatory T cell (Treg).

In some embodiments, at least about 30%, 40%, 50%, 60%, 70%, 80%, 90%, 95%, 99%, 99.5%, or 99.9% of the ammonia oxidizing microorganisms are inactive.

In some embodiments, administration provides for upregulation, activation, downregulation, or suppression of a cytokine associated with an activated immune cell, e.g., IL-5, IL-13, IL-4, IFNγ, IL-12, IL-2, IL-18, IL-17, IL-21, IL-22, IL-10, and TFG-B.

In some embodiments, the preparation is at a temperature greater than about 4° C., when administered.

In some embodiments, the preparation is at a temperature greater than about 10° C., when administered.

In some embodiments, the preparation is at a temperature greater than about room temperature e.g., between about 20° C. - 25° C., when administered.

In some embodiments, a period of time of at least about 1 day, 3 days, 5 days, 1 week, 2 weeks, 3 weeks, 4 weeks, 2 months, 3 months, 4 months, 5 months, 6 months, 1 year, or 5 years has elapsed between packaging and administration.

In some embodiments, the subject is identified as having a disease or disorder modulated by an activated immune cell.

In some embodiments, treatment comprises providing a therapeutic effect for a disease or disorder modulated by an activated immune cell.

In some embodiments, the subject is identified as having a T helper type 1 (Th1) cell mediated disease.

In some embodiments, the Th1 mediated disease is Celiac disease, multiple sclerosis, or diabetes, e.g., type 1 diabetes.

In some embodiments, treatment comprises providing a therapeutic effect for a Th1 mediated disease or a symptom thereof.

In some embodiments, the subject is identified as having a T helper type 2 (Th2) cell mediated disease or disorder.

In some embodiments, the Th2 mediated disease or disorder is associated with atopic dermatitis, allergic rhinitis, asthma, or itch.

In some embodiments, treatment comprises providing a therapeutic effect for a Th2 mediated disease or a symptom thereof.

In some embodiments, the subject is identified as having a T helper type 17 (Th17) cell mediated disease or disorder.

In some embodiments, the Th17 mediated disease or disorder is Autosomal dominant hyper-IgE syndrome (AD-HIES), rheumatoid arthritis, or irritable bowel syndrome.

In some embodiments, treatment comprises providing a therapeutic effect for a Th17 mediated disease or a symptom thereof.

In some embodiments, the subject is identified as having a regulatory T cell (Treg) cell mediated disease or disorder.

In some embodiments, the Treg mediated disease or disorder is immunodysregulation polyendocrinopathy enteropathy-X-linked (IPEX).

In some embodiments, treatment comprises providing a therapeutic effect for a Treg mediated disease or a symptom thereof.

In some embodiments, the preparation is formulated as a spray, aerosol, or mist.

In some embodiments, the preparation is formulated as a powder, cream, ointment, or lotion.

In some embodiments, the preparation comprises a thickener and/or emulsifier.

In some embodiments, the preparation has a viscosity of at least about 1 mPa·s, 10 mPa·s, 100 mPa·s, 1 Pa·s, 5 Pa·s, 10 Pa·s, or 20 Pa·s at room temperature, e.g., between about 20° C. - 25° C.

In some embodiments, the preparation comprises a talcum powder or cornstarch.

In some embodiments, the preparation comprises a component that is toxic to AOM, e.g., an antimicrobial or a preservative, e.g., a preservative listed in Annex VI.

The method may comprise combining the preparation with at least one preservative listed in Annex VI.

The method may comprise combining the preparation with at least 500 ppb of the at least one preservative listed in Annex VI.

In some embodiments, the preparation or formulation is administered topically.

In some embodiments, the preparation or formulation is administered to the body of the subject, e.g., to one or more of the face, neck, scalp, limb, hand, foot, back, buttock, torso, genitals, and chest of the subject.

In some embodiments, the preparation or formulation is administered intranasally.

The method may comprise administering the preparation or formulation to the subject orally, enterally, intranasally, parenterally, subcutaneously, ocularly, otically, or respiratorilly.

In some embodiments, the preparation comprises AOM in a buffer solution, e.g., an aqueous buffer solution.

In some embodiments, the buffer solution, e.g., aqueous buffer solution, comprises disodium phosphate and magnesium chloride, for example, 50 mM Na₂HPO₄ and 2 mM MgCl₂ in water.

In some embodiments, the buffer solution e.g., aqueous buffer solution, consisting essentially of disodium phosphate and magnesium chloride, for example, 50 mM Na₂HPO₄ and 2 mM MgCl₂ in water.

In some embodiments, the buffer solution, e.g., aqueous buffer solution, consists of disodium phosphate and magnesium chloride, for example, 50 mM Na₂HPO₄ and 2 mM MgCl₂ in water.

In some embodiments, the AOM comprise ammonia oxidizing bacteria (AOB).

In some embodiments, the AOM consist essentially of AOB.

In some embodiments, the AOM consist of AOB.

In some embodiments, the AOM comprise Nitrosomonas, Nitrosococcus, Nitrosospira, Nitrosocystis, Nitrosolobus, Nitrosovibrio, and combinations thereof.

In some embodiments, the AOM is Nitrosomonas eutropha (N. eutropha).

In some embodiments, the AOM is N.eutropha D23, having ATCC accession number PTA-121157.

In some embodiments, the AOM comprise ammonia oxidizing archaea (AOA).

Administration may provide for treatment of one or more of: headaches, cardiovascular diseases, inflammation, immune responses, autoimmune disorders, liver diseases, infections, neurological diseases, psychiatric disorders, pulmonary diseases, nitric oxide disorders, urea cycle disorders, congestion, vasodilation disorders, skin diseases, ophthalmic disorders, bowel disorders, auditory diseases, wound healing, reactions to insect bites, connective tissue disorders, and certain viral, bacterial, or fungal infections.

Administration may provide for treatment or improvement of a local effect.

Administration may provide for treatment or improvement of a systemic effect.

The method may comprise obtaining the preparation comprising ammonia oxidizing microorganisms (AOM). The method may comprise preparing a cosmetic, therapeutic, or consumer product from the preparation. The method may comprise measuring at least one of AOM metabolic activity and Th1, Th2, Th17, or Treg inhibition activity of the AOM in the preparation or product to provide an activity value. The method may comprise comparing the activity value to a range of pre-determined values corresponding to a pre-determined range of amounts of AOM metabolic activity and Th1, Th2, Th17, or Treg inhibition activity. The method may comprise determining if the activity value is a value in the range of pre-determined values. In some embodiments, if the activity value is in the range of pre-determined values, the method may comprise classifying the preparation or product as accepted. In some embodiments, if the activity value is outside the range of pre-determined values, the method may comprise classifying the preparation or product as not accepted.

In some embodiments, the method may comprise heat killing a target percentage of the ammonia oxidizing microorganisms if the preparation is not accepted.

In accordance with another aspect, there is provided a method of treating a subject, comprising administering to the subject a therapeutically effective amount of a preparation comprising ammonia oxidizing microorganisms (AOM). In some embodiments, the preparation was distributed by a method of any of the preceding claims.

In accordance with another aspect, there is provided a shelf-stable preparation. The shelf-stable preparation may comprise at least about 10³ cells/mL of ammonia oxidizing microorganisms (AOM). In some embodiments, less than about 70%, 60%, 50%, 40%, 30%, 20%, 10%, 5%, 1%, 0.5%, or 0.1% of the ammonia oxidizing microorganisms are viable. In some embodiments, the preparation has a viscosity of at least about 1 mPa·s, 10 mPa·s, 100 mPa·s, 1 Pa·s, 5 Pa·s, 10 Pa·s, or 20 Pa·s at room temperature, e.g., between about 20° C. - 25° C. In some embodiments, the preparation is formulated as a powder, cream, ointment, salve, or lotion. In some embodiments, the preparation comprises a component that is toxic to AOM, e.g., an antimicrobial or preservative, e.g., a preservative listed in Annex VI. In some embodiments, the preparation has been sterilized.

In accordance with another aspect, there is provided a shelf-stable preparation. The shelf-stable preparation may comprise at least about 10³ CFU/mL in 750-1000 mg of ammonia oxidizing microorganisms (AOM). The shelf-stable preparation may comprise at least one preservative listed in Annex VI. In particular, the shelf-stable preparation may comprise at least 500 ppb of the at least one preservative.

In accordance with another aspect, there is provided a shelf-stable preparation. The shelf-stable preparation may comprise at least about 10³ CFU/mL in 750-1000 mg of ammonia oxidizing microorganisms (AOM). The shelf-stable preparation may comprise at least one preservative listed in Annex VI. In some embodiments, the preparation if exposed to challenge with a pathogenic microorganism, will not support growth of the pathogenic microorganism.

In some embodiments, the preparation comprises at least 500 ppb of the at least one preservative listed in Annex VI.

In some embodiments, the preparation comprises a component that is toxic to AOM, e.g., an antimicrobial.

In some embodiments, the preparation comprises at least about 10³ cells/mL, 10⁴ cells/mL, 10⁵ cells/mL, or 10⁶ cells/mL.

In some embodiments, the preparation comprises at least about 10³ CFU/mL, 10⁴ CFU/mL, 10⁵ CFU/mL, or 10⁶ CFU/mL.

In some embodiments, the preparation has been sterilized.

In some embodiments, the preparation is substantially free of polyphosphate.

In some embodiments, the preparation if exposed to challenge with a population of a pathogenic microorganism, will sterilize at least 30%, 40%, 50%, 60%, 70%, 80%, 90%, 95%, or 99% of the population of the microorganism.

In some embodiments, the preparation is formulated as a liquid, e.g., spray, aerosol, or mist.

In some embodiments, the preparation is formulated as a powder, cream, ointment, salve, or lotion.

In some embodiments, the preparation comprises a thickener and/or emulsifier.

In some embodiments, the preparation has a viscosity of at least about 1 mPa·s, 10 mPa·s, 100 mPa·s, 1 Pa·s, 5 Pa·s, 10 Pa·s, or 20 Pa·s at room temperature, e.g., between about 20° C. - 25° C.

In some embodiments, the preparation comprises a talcum powder or cornstarch.

In some embodiments, the preparation is packaged in an end use container.

The end use container may indicate one or more of the following: storage and handling of the preparation, formulation of the preparation, description of contents in the preparation, viability status of the AOM, and directions for use of the preparation.

In other embodiments, the end use container does not indicate one or more of the following: storage and handling of the preparation, formulation of the preparation, description of contents in the preparation, viability status of the AOM, and directions for use of the preparation.

In some embodiments, the end use container informs the subject to apply the preparation topically.

In some embodiments, the end use container informs the subject to apply the preparation intranasally.

In some embodiments, the end use container informs the subject to apply the preparation at least one of orally, enterally, intranasally, parenterally, subcutaneously, ocularly, otically, or respiratorilly.

In some embodiments, the end use container, comprises a polymer bottle, e.g., a spray, aerosol, or mist bottle.

In some embodiments, the end use container, comprises a squeezable container, e.g., squeeze bottle or tube.

In some embodiments, wherein the end use container, is substantially free of a vacuum bag.

In some embodiments, the end use container, is not configured to inhibit or reduce retrograde flow.

In some embodiments, the end use container comprises polymer, e.g., polyethylene terephthalate (PET), high density polyethylene (HDPE), polypropylene, polycarbonate, polytetrafluoroethylene (teflon®), polyviylidene fluoride (PVDF), or a cellulosic, glass, aluminum, or cardboard.

In some embodiments, the end use container, is configured to allow passage of oxygen.

In some embodiments, the end use container, is configured to allow passage of at least about 5, 10, 20, 30, 40, 50, 60, 70, 80, 90, 95, 99, or 100 percent of transmission of ionizing radiation, e.g., with gamma rays, e.g., with x-rays, e.g., from an isotope, e.g., cobalt 60, or with ultraviolet, e.g., ultraviolet C (UVC) through the container.

The preparation may be formulated for oral, enteral (e.g., buccal, sublingual, sublabial, and rectal), parenteral (e.g., subcutaneous, intradermal, intramuscular, intravenous, and intraarticular), inhalation (e.g., fine particle dusts or mists which may be generated by means of various types of metered doses, pressurized aerosols, nebulizers or insufflators, and including intranasally or via the lungs), intranasal, eye, ear, rectal, injection, urogenital, or topical (e.g., dermal, transdermal, transmucosal, buccal, sublingual, and intraocular) administration.

The preparation may be formulated for treatment of one or more of: headaches, cardiovascular diseases, inflammation, immune responses, autoimmune disorders, liver diseases, infections, neurological diseases, psychiatric disorders, nitric oxide disorders, urea cycle disorders, congestion, vasodilation disorders, skin diseases, ophthalmic disorders, wound healing, reactions to insect bites, connective tissue disorders, and certain viral, bacterial, or fungal infections.

The preparation may be formulated for treatment of a disease or disorder modulated by an activated immune cell.

In some embodiments, the activated immune cell is a T helper cell or regulatory T cell, e.g., T helper type 1 (Th1), T helper type 2 (Th2), T helper type 17 (Th17), or regulatory T cell (Treg).

In some embodiments, the preparation is formulated for upregulation, activation, downregulation, or suppression of a cytokine associated with an activated immune cell, e.g., IL-5, IL-13, IL-4, IFNγ, IL-12, IL-2, IL-18, IL-17, IL-21, IL-22, IL-10, and TFG-B.

In some embodiments, the preparation is formulated for treatment of a Th1 mediated disease or disorder, e.g., Celiac disease, multiple sclerosis, or diabetes, e.g., type 1 diabetes.

In some embodiments, the preparation is formulated for treatment of a Th2 mediated disease or disorder, e.g., atopic dermatitis, allergic rhinitis, asthma, or itch.

In some embodiments, the preparation is formulated for treatment of a Th17 mediated disease or disorder, e.g., Job’s syndrome, rheumatoid arthritis, irritable bowel syndrome.

In some embodiments, the preparation is formulated for treatment of a Treg mediated disease or disorder, e.g., immunodysregulation polyendocrinopathy enteropathy-X-linked (IPEX).

In some embodiments, the preparation comprises AOM in a buffer solution, e.g., an aqueous buffer solution.

In some embodiments, the buffer solution, e.g., aqueous buffer solution, comprises disodium phosphate and magnesium chloride, for example, 50 mM Na₂HPO₄ and 2 mM MgCl₂ in water.

In some embodiments, the buffer solution e.g., aqueous buffer solution, consisting essentially of disodium phosphate and magnesium chloride, for example, 50 mM Na₂HPO₄ and 2 mM MgCl₂ in water.

In some embodiments, the buffer solution, e.g., aqueous buffer solution, consists of disodium phosphate and magnesium chloride, for example, 50 mM Na₂HPO₄ and 2 mM MgCl₂ in water.

In some embodiments, the AOM comprise ammonia oxidizing bacteria (AOB).

In some embodiments, the AOM consist essentially of AOB.

In some embodiments, the AOM consist of AOB.

In some embodiments, the AOM comprise Nitrosomonas, Nitrosococcus, Nitrosospira, Nitrosocystis, Nitrosolobus, Nitrosovibrio, and combinations thereof.

In some embodiments, the AOM is Nitrosomonas eutropha (N. eutropha).

In some embodiments, the AOM is N.eutropha D23, having ATCC accession number PTA-121157.

In some embodiments, less than about 70%, 60%, 50%, 40%, 30%, 20%, 10%, 5%, 1%, 0.5%, or 0.1% of the ammonia oxidizing microorganisms are viable.

In some embodiments, at least about 30%, 40%, 50%, 60%, 70%, 80%, 90%, 95%, 99%, 99.5%, or 99.9% of the ammonia oxidizing microorganisms are inactive.

In accordance with another aspect, there is provided a method of treating a subject comprising administering to the subject a therapeutically effective amount of the preparation of any of the preceding claims.

The disclosure contemplates all combinations of any one or more of the foregoing aspects and/or embodiments, as well as combinations with any one or more of the embodiments set forth in the detailed description and any examples.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a graph of D23 metabolic activity for live D23 and heat killed D23;

FIG. 2A is a graph of PBMC percent activity and viability for PBMCs co-cultured with D23 or heat killed D23 in the presence or absence of a Th2 stimulus;

FIG. 2B is a graph of nitrite production by PBMCs, D23, and PBMCs co-cultured with D23;

FIG. 3A is a graph of IL-5 expression for Th2 stimulated PBMCs co-cultured with D23 and heat killed D23;

FIG. 3B is a graph of IL-13 expression for Th2 stimulated PBMCs co-cultured with D23 and heat killed D23;

FIG. 3C is a graph of relative IL-4 expression for Th2 stimulated PBMCs co-cultured with D23 and heat killed D23;

FIG. 4A is a graph of IL-5 expression for Th2 stimulated PBMCs co-cultured with D23 and heat killed D23;

FIG. 4B is a graph of IL-13 expression for Th2 stimulated PBMCs co-cultured with D23 and heat killed D23;

FIG. 5A is a graph of TNFα expression for unstimulated cells co-cultured with D23 and heat killed D23;

FIG. 5B is a graph of TNFα expression for LpS stimulated cells co-cultured with D23 and heat killed D23;

FIG. 5C is a graph of TNFα expression for CD3/28 stimulated cells co-cultured with D23 and heat killed D23;

FIG. 6A is a graph of IL-6 expression for unstimulated cells co-cultured with D23 and heat killed D23;

FIG. 6B is a graph of IL-6 expression for LpS stimulated cells co-cultured with D23 and heat killed D23;

FIG. 6C is a graph of IL-6 expression for CD3/28 stimulated cells co-cultured with D23 and heat killed D23;

FIG. 7A is a graph of IL-10 expression for unstimulated cells co-cultured with D23 and heat killed D23;

FIG. 7B is a graph of IL-10 expression for LpS stimulated cells co-cultured with D23 and heat killed D23;

FIG. 7C is a graph of IL-10 expression for CD3/28 stimulated cells co-cultured with D23 and heat killed D23;

FIG. 8A is a graph of IFNγ expression for unstimulated cells co-cultured with D23 and heat killed D23;

FIG. 8B is a graph of IFNγ expression for LpS stimulated cells co-cultured with D23 and heat killed D23;

FIG. 8C is a graph of IFNγ expression for CD3/28 stimulated cells co-cultured with D23 and heat killed D23;

FIG. 9A is a graph of IL-2 expression for unstimulated cells co-cultured with D23 and heat killed D23;

FIG. 9B is a graph of IL-2 expression for LpS stimulated cells co-cultured with D23 and heat killed D23;

FIG. 9C is a graph of IL-2 expression for CD3/28 stimulated cells co-cultured with D23 and heat killed D23;

FIG. 10A is a graph of AOB metabolic activity (expressed as nitrite production) for live AOB and heat killed AOB;

FIG. 10B is a graph of AOB metabolic activity (expressed as nitric oxide production) for live AOB and heat killed AOB;

FIG. 10C is a graph of fold reduction in IL-5 expression mediated by AOB and heat killed AOB;

FIG. 10D is a graph of fold reduction in IL-13 expression mediated by AOB and heat killed AOB;

FIG. 10E is a graph of fold reduction in MHC II expression mediated by AOB and heat killed AOB;

FIG. 10F is a graph of fold reduction in CD86 expression mediated by AOB and heat killed AOB;

FIG. 11 is a graph of fold reduction in IL-5 expression mediated by D23 with TLR1, TLR4, TLR6, TLR5, TLR2, TLR9, and TLR8 inhibitor;

FIG. 12 is a graph of fold reduction in IL-5 expression mediated by D23 with TLR8 inhibitor;

FIG. 13A is a graph of the expression of IFNγ for Th2 cells, Th2 cells co-cultured with AOB, and Th2 cells co-cultured with heat killed AOB;

FIG. 13B is a graph of the expression of IL-12 for Th2 cells, Th2 cells co-cultured with AOB, and Th2 cells co-cultured with heat killed AOB;

FIG. 14 is a graph of IL-5 expression for live D23 cells, heat killed cells, aged cells, cells treated by freeze/thaw cycles, and ethanol treated cells, according to one embodiment;

FIG. 15A is a graph of Eczema Area and Severity Index (EASI) score after administration of a shelf-stable composition, according to one embodiment;

FIG. 15B is a graph of Investigator’s Global Assessment (IGA) score after administration of a shelf-stable composition, according to one embodiment;

FIG. 16 is a graph showing the percentage of subjects with improved EASI and IGA scores from baseline after administration of a shelf-stable composition, according to one embodiment;

FIG. 17 is a graph of visual analogue scale (VAS) score for itch after administration of a shelf-stable composition, according to one embodiment;

FIG. 18 is a graph of itchman score after administration of a shelf-stable composition, according to one embodiment;

FIG. 19A is a graph showing the percentage of subjects with improved VAS score from baseline after administration of a shelf-stable composition, according to one embodiment; and

FIG. 19B is a graph showing the percentage of subjects with improved itchman scores from baseline after administration of a shelf-stable composition, according to one embodiment.

### DETAILED DESCRIPTION

In accordance with one or more embodiments, the present disclosure provides for various methods or modes of introducing ammonia oxidizing microorganisms to a subject. These methods or modes comprise administering to a subject ammonia oxidizing microorganisms, for example, a preparation, composition, formulation, or product comprising ammonia oxidizing microorganisms. In at least some embodiments, ammonia oxidizing microorganisms may therefore generally be restored to a microbiome of the subject. In at least some embodiments, the ammonia oxidizing microorganism preparations, compositions, formulations described herein may be shelf-stable.

Preparations, compositions, and/or formulations, e.g., including cosmetic products, therapeutic products, consumer products, non-natural products, natural products, and fortified natural products, comprising, consisting essentially of, or consisting of ammonia oxidizing microorganisms are disclosed. These preparations, compositions, and/or formulations are disclosed herein for use in various applications, e.g., cosmetic and/or therapeutic applications. The preparations, compositions, and/or formulations may be administered in an effective amount for an intended use, e.g., a cosmetic or a therapeutic application. Preparations, compositions, and/or formulations comprising ammonia oxidizing microorganisms for various modes of administration to a subject are provided. Preparations, compositions, and/or formulations comprising ammonia oxidizing microorganisms for use in the treatment of various conditions and/or disorders in a subject are provided. Methods of treating a subject for various conditions and/or disorders via administration of ammonia oxidizing microorganisms are disclosed. Devices for use in administering ammonia oxidizing microorganisms to a subject are also provided.

### Microbiology

In accordance with one or more embodiments, essentially any ammonia oxidizing microorganism (AOM) can be used or implemented. The ammonia oxidizing microorganisms may generally be autotrophic. The ammonia oxidizing microorganisms may generate nitrite and/or nitric oxide from ammonia.

Properties of autotrophic ammonia oxidizing bacteria (AOB), for example, are well described by Whitlock in U.S. Pat. No. 7,820,420. Since that filing, the class of autotrophic microorganisms that oxidize ammonia for ATP production has been expanded to encompass ammonia oxidizing archaea (AOA), and archaea have been moved out of the class of bacteria and into their own distinct class. For the purposes of this disclosure, any and all autotrophic ammonia oxidizing microorganisms that share the properties of oxidation of ammonia to generate ATP can be implemented. AOM, including both AOB and AOA, share the necessary properties of oxidation of ammonia into NO and nitrite and all known AOM lack capacity for virulence because of their inability to use organic substrates for ATP generation. Bacteria can utilize ammonia at higher concentrations, while archaea can utilize ammonia at lower concentrations. Physiological levels of ammonia are within the range that both bacteria (AOB) and archaea (AOA) can utilize. Any reference specifically to ammonia oxidizing bacteria throughout this disclosure should be considered equally applicable to any ammonia oxidizing microorganism, e.g., any ammonia oxidizing archaea, and these terms may all be used interchangeably herein.

Ammonia oxidizing bacteria (AOB) are ubiquitous Gram-negative obligate bacteria with a unique capacity to generate energy exclusively from the conversion of ammonia to nitrite. In some embodiments, ammonia oxidizing bacteria (AOB) of the genus Nitrosomonas are Gram-negative obligate autotrophic (chemolithoautotrophic) bacteria with a unique capacity to generate nitrite and nitric oxide exclusively from ammonia as an energy source. They are widely present both in soil and water environments and are essential components of environmental nitrification processes. These bacteria have beneficial properties, e.g., in connection with various cosmetic and therapeutic uses, in accordance with one or more embodiments described herein. Without wishing to be bound to any particular theory, due to the roles of nitrite and nitric oxide as important components of several physiological functions, such as vasodilation, inflammation and wound healing, these bacteria may have various beneficial properties for both healthy and immunopathological conditions. These bacteria are safe for use in humans because they are slow-growing, cannot grow on organic carbon sources, may be sensitive to soaps and antibiotics, and have never been associated with any disease or infection in animals or humans.

Ammonia oxidizing microorganisms generate coenzyme Q 8 (CoQ8) as a byproduct of the process by which they generate nitrite and nitric oxide. CoQ8 is a coenzyme Q having 8 carbons in its isoprenoid side chain. Without wishing to be bound to any particular theory, due to the role of coenzyme Q as an important component of several cell functions, such as mediating cell signaling and preventing cell death (anti-aging), these microorganisms’ beneficial properties may further be enhanced by their specific ability to generate CoQ8.

In some embodiments, ammonia oxidizing bacteria may catalyze the following reactions.

At a neutral pH level, ammonia generated from ammonium around neutral pH conditions is the substrate of the initial reaction. The conversion of ammonia to nitrite takes place in two steps catalyzed respectively by ammonia monooxygenase (AMO) and hydroxylamine oxidoreductase (HAO), as follows:

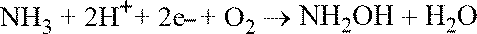

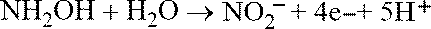

In some instances, reaction B is reported as follows, to indicate nitrous acid (HNO₂) formation at low pH:

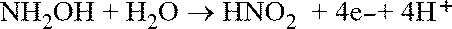

In certain embodiments, NH₄⁺ and NH₃ may be used interchangeably throughout the disclosure.

Examples of ammonia oxidizing bacteria include Nitrosomonas eutropha strains, e.g., D23 and C91 as discussed herein, and other bacteria in the genera Nitrosomonas, Nitrosococcus, Nitrosospira, Nitrosocystis, Nitrosolobus, and Nitrosovibrio. D23 Nitrosomonas eutropha strain refers to the strain, designated AOB D23-100, deposited with the American Tissue Culture Collection (ATCC) (10801 University Blvd., Manassas, VA, USA) on Apr. 8, 2014 having accession number PTA-121157. The nucleic acid sequence(s), e.g., genome sequence, of accession number PTA-121157 are hereby incorporated herein by reference in their entireties for all purposes. “AOB D23-100” may also be referred to as D23 or B244 throughout this disclosure.

Examples of ammonia oxidizing archaea include archaea in the genera Methanobrevibacter, Methanosphaera, Methanosarcina, Nitroscaldus, Nitrosopumilus, and Nitrososphaera (e.g. Nitrososphaera viennensis, Nitrososphaera gargensis). Different phylotypes of archaea, e.g., methanogens and halphilic archaeon, may be included in the preparations disclosed herein. Examples of archaea further include archaea in the lineages of phyla Euryarchaeota (e.g. Methanosarcina), Crenarchaeota, Aigarchaeota, and Thaumarchaeota (e.g. Giganthauma karukerense, Giganthauma insulaporcus, Caldiarchaeum subterraneum, Cenarchaeum symbiosum).

Each and every nucleic acid sequence and amino acid sequence disclosed in International (PCT) Patent Application Publication No. WO2015/160911 (International (PCT) Patent Application Serial No. PCT/US2015/025909 as filed on Apr. 15, 2015), is hereby incorporated herein by reference in its entirety for all purposes. Likewise, any ammonia oxidizing bacteria disclosed in International (PCT) Patent Application Publication No. WO2015/160911 (International (PCT) Patent Application Serial No. PCT/US2015/025909 as filed on Apr. 15, 2015), is also hereby incorporated herein by reference in its entirety for all purposes. In certain embodiments, the ammonia oxidizing microorganism is a strain as described therein.

In accordance with one or more embodiments, ammonia oxidizing microorganisms may exist in several metabolic states, e.g. growth state, storage state, and/or polyphosphate loading state.

In accordance with one or more embodiments, ammonia oxidizing microorganisms may have desirable properties, e.g., optimized properties, such as the ability to suppress growth of pathogenic bacteria, and an enhanced ability to produce nitric oxide and nitric oxide precursors.

Optimized Nitrosomonas eutropha (N. eutropha), as that term is used herein, refers to an N. eutropha having an optimized growth rate; an optimized NH₄⁺ oxidation rate; and/or optimized resistance to NH₄⁺. In an embodiment it differs from naturally occurring N. eutropha by at least one nucleotide, e.g., a nucleotide in a gene selected from ammonia monooxygenase, hydroxylamine oxidoreductase, cytochrome c554, and cytochrome c_{M}552. The difference can arise, e.g., through selection of spontaneously arising mutation, induced mutation, or directed genetic engineering, of the N. eutropha. In an embodiment it differs from a naturally occurring N. eutropha in that it has a constellation of alleles, not present together in nature. These differences may provide for one or more of a treatment or prevention of a disease or condition, such as but not limited to one associated with low nitrite levels.

Any ammonia oxidizing bacteria, e.g., N. eutropha, for example N. eutropha referred to as “D23”, also known as “B244” or “AOB D23-100” may have several of the above-described properties. Any ammonia oxidizing archaea (AOA) may also have several of the above-described properties.

The AOBs contemplated in this disclosure may comprise mutations relative to wild-type AOBs. These mutations may, e.g., occur spontaneously, be introduced by random mutagenesis, or be introduced by targeted mutagenesis. For instance, the AOBs may lack one or more genes or regulatory DNA sequences that wild-type AOBs typically comprise. The AOBs may also comprise point mutations, substitutions, insertions, deletions, and/or rearrangements relative to the sequenced strain or a wild-type strain. The AOBs may be a purified preparation of optimized AOBs.

In certain embodiments, the AOBs are transgenic. For instance, it may comprise one or more genes or regulatory DNA sequences that wild-type ammonia oxidizing bacteria lacks. More particularly, the ammonia oxidizing bacteria may comprise, for instance, a reporter gene, a selective marker, a gene encoding an enzyme, or a promoter (including an inducible or repressible promoter). In some embodiments the additional gene or regulatory DNA sequence is integrated into the bacterial chromosome; in some embodiments the additional gene or regulatory DNA sequence is situated on a plasmid.

In some embodiments, the AOBs differ by at least one nucleotide from naturally occurring bacteria. For instance, the AOBs may differ from naturally occurring bacteria in a gene or protein that is part of a relevant pathway, e.g., an ammonia metabolism pathway, a urea metabolism pathway, or a pathway for producing nitric oxide or nitric oxide precursors. More particularly, the AOBs may comprise a mutation that elevates activity of the pathway, e.g., by increasing levels or activity of an element of that pathway.

The above-mentioned mutations can be introduced using any suitable technique. Numerous methods are known for introducing mutations into a given position. For instance, one could use site-directed mutagenesis, oligonucleotide-directed mutagenesis, or site-specific mutagenesis. Non-limiting examples of specific mutagenesis protocols are described in, e.g., Mutagenesis, pp. 13.1-13.105 (Sambrook and Russell, eds., Molecular Cloning A Laboratory Manual, Vol. 3, 3.sup.rd ed. 2001). In addition, non-limiting examples of well-characterized mutagenesis protocols available from commercial vendors include, without limitation, Altered Sites.RTM. II in vitro Mutagenesis Systems (Promega Corp., Madison, Wis.); Erase-aBase.RTM. System (Promega, Madison, Wis.); GeneTailor.TM. Site-Directed Mutagenesis System (Invitrogen, Inc., Carlsbad, Calif.); QuikChange.RTM. II Site-Directed Mutagenesis Kits (Stratagene, La Jolla, Calif.); and Transformer.TM. Site-Directed Mutagenesis Kit (BD-Clontech, Mountain View, Calif.).

In certain embodiments of the disclosure, the ammonia oxidizing microorganisms may be axenic. The preparation (formulation or composition) of ammonia oxidizing microorganisms may comprise, consist essentially of, or consist of axenic ammonia oxidizing microorganisms.

The ammonia oxidizing bacteria of this disclosure may be from a genus selected from the group consisting of Nitrosomonas, Nitrosococcus, Nitrosospria, Nitrosocystis, Nitrosolobus, Nitrosovibrio, and combinations thereof.

This disclosure provides, inter alia, N. eutropha strain D23, a unique, e.g., optimized strain of ammonia oxidizing bacteria that can increase production of nitric oxide and nitric oxide precursors on a surface of a subject, e.g., a human subject. This disclosure also provides methods of administering and using the bacteria and preparations, compositions, formulations, and products, comprising the bacteria.

In embodiments, the ammonia oxidizing bacteria, e.g., N. eutropha is non-naturally occurring. For instance, it may have accumulated desirable mutations during a period of selection. In other embodiments, desirable mutations may be introduced by an experimenter. In some embodiments, the N. eutropha may be a purified preparation, and may be an optimized N.eutropha.

In preferred embodiments, the N. eutropha strain is autotrophic and so incapable of causing infection. A preferred strain utilizes urea as well as ammonia, so that hydrolysis of the urea in sweat would not be necessary prior to absorption and utilization by the bacteria. Also, in order to grow at low pH, the bacteria may either absorb NH₄⁺ ions or urea. The selected strain should also be capable of living on the external skin of a subject, e.g., a human, and be tolerant of conditions there.

Although this disclosure refers to N. eutropha strain D23 in detail, the preparations, methods, compositions, treatments, formulas and products may be used with one or more of: one or more other strains of N. eutropha, one or more other species of Nitrosomonas, and one or more other ammonia oxidizing microorganism, e.g. ammonia oxidizing bacteria or other ammonia oxidizing archaea.

In certain embodiments, a bacterium with the above-mentioned sequence characteristics has one or more of (1) an optimized growth rate as measured by doubling time, (2) an optimized growth rate as measured by OD600, (3) an optimized NH₄⁺ oxidation rate, (4) an optimized resistance to NH₄⁺, and (4) an optimized resistance to NO₂⁻. Particular nonlimiting subcombinations of these properties are specified in the following paragraph.

In some embodiments, the ammonia oxidizing bacteria, e.g., the N.eutropha described herein, or an axenic composition thereof, has one or more of: (1) an optimized growth rate as measured by doubling time, (2) an optimized growth rate as measured by OD600, (3) an optimized NH₄⁺ oxidation rate, (4) an optimized resistance to, NH₄⁺, and (4) an optimized resistance to, NO₂⁻. For instance, the bacterium may have properties (1) and (2); (2) and (3); (3) and (4); or (4) and (5) from the list at the beginning of this paragraph. As another example, the bacterium may have properties (1), (2), and (3); (1), (2), and (4); (1), (2), and (5); (1), (3), and (4); (1), (3), and (5); (1), (4), and (5); (2), (3), and (4); (2), (3), and (5), or (3), (4), and (5) from the list at the beginning of this paragraph. As a further example, the bacterium may have properties (1), (2), (3), and (4); (1), (2), (3), and (5); (1), (2), (4), and (5); (1), (3), (4), and (5); or (2), (3), (4), and (5) from the list at the beginning of this paragraph. In some embodiments, the bacterium has properties (1), (2), (3), (4), and (5) from the list at the beginning of this paragraph.

In certain embodiments, the N. eutropha strain comprises a nucleic acid sequence, e.g., a genome, that hybridizes to SEQ ID NO: 1 of International (PCT) Patent Application Publication No. WO2015160911 (International (PCT) Patent Application Serial No. PCT/US2015/025909 filed on Apr. 15, 2015), or to the genome of the D23 strain deposited in the form of 25 vials with the ATCC patent depository on Apr. 8, 2014, designated AOB D23-100, under accession number PTA-121157, or their complements, under low stringency, medium stringency, high stringency, or very high stringency, or other hybridization condition.

The D23 strain is not believed to be a product of nature, but rather has acquired certain mutations and characteristics during an extended period of culture and selection in the laboratory. For instance, D23 has an ability to grow in conditions of greater than about 200 or 250 mM NH₄⁺ for more than 24 hours.

In some embodiments, the N. eutropha disclosed herein differ from naturally occurring bacteria in the abundance of siderophores. For instance, the N. eutropha may have elevated or reduced levels of siderophores compared to N. eutropha C91. Generally, siderophores are secreted iron-chelating compounds that help bacteria scavenge iron from their environment. Some siderophores are peptides, and others are small organic molecules.

The practice of the present invention may employ, unless otherwise indicated, conventional methods of immunology, molecular biology, and recombinant DNA techniques within the skill of the art. Such techniques are explained fully in the literature. See, e.g., Sambrook, et al. Molecular Cloning: A Laboratory Manual (Current Edition); and Current Protocols in Molecular Biology (F.M. Ausubel, et al. eds., current edition).

### Select Definitions

An ammonia oxidizing microorganism, e.g., ammonia oxidizing bacteria, refers to a microorganism capable of oxidizing ammonia or ammonium to nitrite at a rate, e.g., a substantial rate, e.g., a pre-determined rate. The rate, e.g., a pre-determined rate, may refer to the conversion of ammonium ions (NH₄⁺) (e.g., at about 200 mM) to nitrite (NO₂⁻), for example, as determined or measured in an in vitro assay or when administered to a subject, e.g., a human. The rate may be a conversion at a rate of at least about 1 picomole per minute per mg protein, 0.01, 0.1, 1, 10, 25, 50, 75, 125, or 150 nanomoles NO₂⁻ per minute per mg protein, e.g., about 0.01-1, 0.1-50, 50-100, 100-150, 75-175, 75-125, 100-125, 125-150, or 125-175 nanomoles/minute/mg protein, e.g., about 125 nanomoles NO₂⁻ per minute per mg protein for a continuous culture, for example having an OD of about 0.5. The rate of conversion may be between about 1 picomole per minute per mg protein to about 1 millimole per minute per mg protein. The rate of conversion may be at most about 1 mole NO₂⁻ per minute per mg protein, e.g. at least about, about, or at most about 1 decimole, 1 centimole, 1 millimole, or 1 micromole NO₂⁻ per minute per mg protein.

As used herein, “axenic” refers to a composition comprising an organism that is substantially free of other organisms. For example, an axenic culture of ammonia oxidizing bacteria is a culture that is substantially free of organisms other than ammonia oxidizing bacteria. For example, an axenic culture of N. eutropha is a culture that is substantially free of organisms other than N. eutropha. In some embodiments, “substantially free” denotes undetectable by a method used to detect other organisms, e.g., plating the culture and examining colony morphology, or PCR for a conserved gene such as 16S RNA. An axenic composition may comprise elements that are not organisms, e.g., it may comprise nutrients or excipients. Any embodiment, preparation, composition, or formulation of ammonia oxidizing bacteria discussed herein may comprise, consist essentially of, or consist of optionally axenic ammonia oxidizing bacteria.

Throughout this disclosure, formulation may refer to a composition or preparation or product.

As used herein, an “autotroph”, e.g., an autotrophic bacterium, is any organism capable of self-nourishment by using inorganic materials as a source of nutrients and using photosynthesis or chemosynthesis as a source of energy. Autotrophic bacteria may synthesize organic compounds from carbon dioxide and ATP derived from other sources, oxidation of ammonia to nitrite, oxidation of hydrogen sulfide, and oxidation of Fe²⁺ to Fe³⁺. Autotrophic bacteria of the present disclosure are incapable of causing infection.

Administered “in combination,” as used herein, means that two (or more) different treatments are delivered to the subject during the course of the subject’s affliction with the disorder, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concomitant” or “concurrent delivery”. In other embodiments, the delivery of one treatment ends before the delivery of the other treatment begins. This is sometimes referred to herein as “successive” or “sequential delivery.” In embodiments of either case, the treatment is more effective because of combined administration. For example, the second treatment is a more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive (i.e., synergistic). The delivery can be such that an effect of the first treatment delivered is still detectable when the second is delivered. In some embodiments, one or more treatment may be delivered prior to diagnosis of the patient with the disorder.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, the term “optimized growth rate” refers to one or more of: a doubling time of less than about 4, 5, 6, 7, 8, 9, or 10 hours when cultured under batch conditions as described herein in Example 2; a doubling time of less than about 16, 18, 20, 22, 24, or 26 hours, when grown under chemostat conditions as described herein in Example 2; or growing from an OD600 of about 0.15 to at least about 0.3, 0.4, 0.5, 0.6, 0.7, or 0.8 over about 1 or 2 days. In an embodiment, optimized growth rate is one having a doubling time that it is at least 10, 20, 30, 40, or 50% shorter than that of a naturally occurring N. eutropha.

As used herein, “optimized NH₄⁺ oxidation rate” refers to a rate of at least about 50, 75, 125, or 150 micromoles per minute of converting NH₃ or NH₄⁺ into NO₂⁻. For instance, the rate may be at least about 50, 75, 125, or 150 micromoles per minute of converting NH₄⁺ (e.g., at about 200 mM) to NO₂⁻. In an embodiment, an optimized NH₄⁺ oxidation rate is one in which NH₃ or NH₄⁺ is converted into NO₂^{-,} at least 10, 20, 30, 40, or 50% more rapidly than is seen with a naturally occurring N. eutropha.

As used herein, “optimized resistance to NH₄⁺” refers to an ability to grow in conditions of greater than 50, 75, 100, 125, 150, 175, 200, 225, 250, 275, or 300 mM NH₃ or NH₄⁺ for at least about 24 or 48 hours. In an embodiment, an optimized resistance to NH₄⁺ refers to the ability to grow at least 10, 20, 30, 40, or 50% more rapidly, or at least 10, 20, 30, 40, or 50% longer, in the presence of a selected concentration of NH₃ or NH₄⁺ than can a naturally occurring N. eutropha.

As used herein, “transgenic” means comprising one or more exogenous portions of DNA. The exogenous DNA is derived from another organism, e.g., another bacterium, a bacteriophage, an animal, or a plant.

As used herein, treatment of a disease or condition refers to reducing the severity or frequency of at least one symptom of that disease or condition, compared to a similar but untreated patient. Treatment can also refer to halting, slowing, or reversing the progression of a disease or condition, compared to a similar but untreated patient. Treatment may comprise addressing the root cause of the disease and/or one or more symptoms.

As used herein a therapeutically effective amount refers to a dose sufficient to prevent advancement, or to cause regression of a disease or condition, or which is capable of relieving a symptom of a disease or condition, or which is capable of achieving a desired result. A therapeutically effective dose can be measured, for example, as a number of bacteria or number of viable bacteria (e.g., in CFUs) or a mass of bacteria (e.g., in milligrams, grams, or kilograms), or a volume of bacteria (e.g., in mm³).

As used herein, the term “viability” refers to the autotrophic microorganism’s, e.g., ammonia oxidizing microorganism’s, ability to oxidize ammonia, ammonium, or urea to nitrite at a pre-determined rate. In some embodiments, the rate refers to the conversion of ammonium ions (NH₄⁺) (e.g., at about 200 mM) to nitrite (NO₂⁻) at a rate of at least about 1 picomole, 0.01, 0.1, 1, 10, 25, 50, 75, 125, or 150 nanomoles NO₂⁻ per minute, e.g., about 0.01-1, 0.1-50, 50-100, 100-150, 75-175, 75-125, 100-125, 125-150, or 125-175 nanomoles/minute, e.g., about 125 nanomoles NO₂⁻ per minute. The rate of conversion may be at most about 1 mole NO₂⁻ per minute, e.g. at least about, about, or at most about 1 decimole, 1 centimole, 1 millimole, or 1 micromole NO₂⁻ per minute. Viable ammonia oxidizing microorganisms may generally comprise culturable AOMs or AOMs that are otherwise able to generate NO, nitrate, or nitrite.

As used herein, the term “activity” refers to the autotrophic microorganism’s, e.g., ammonia oxidizing microorganism’s, ability to reproduce. The active cells are referred to as colony forming units (CFU). The inactive AOM may be sterilized, dead, or otherwise not viable. In some embodiments, the AOM may be inactivated by time, temperature, or contact with a toxicant, e.g., a preservative. In particular embodiments, the inactive AOM may be heat killed. The inactive AOM may be killed by freeze/thawing, ethanol treatment, or aging. The inactive AOM may comprise one or more structural or intracellular component, e.g., a structural or intracellular component having a cosmetic or therapeutic effect. In some embodiments, the structural or intracellular component may play a role in a signaling cascade, e.g., having an effect on an activated immune cell response, e.g., having an inhibitory effect on an activated immune cell response.

As used herein, a “subject” may include an animal, a mammal, a human, a non-human animal, a livestock animal, or a companion animal. The term “subject” is intended to include human and non-human animals, for example, vertebrates, large animals, and primates. In certain embodiments, the subject is a mammalian subject, and in particular embodiments, the subject is a human subject. Although applications with humans are clearly foreseen, veterinary applications, for example, with non-human animals, are also envisaged herein. The term “non-human animals” of the disclosure includes all vertebrates, for example, non-mammals (such as birds, for example, chickens; amphibians; reptiles) and mammals, such as non-human primates, domesticated, and agriculturally useful animals, for example, sheep, dog, cat, cow, pig, rat, among others.

“Microbiome” refers to a population, e.g, one or more microorganisms that live on a surface of a subject, e.g., in the gut, mouth, skin, and/or elsewhere in a subject. The population may have one or more beneficial functions and/or benefits, relevant to supporting the life of a subject.

“Biome-friendly” refers to something, e.g, a product, e.g., a cosmetic product, e.g., a finished cosmetic product that may allow for minimal disruption of a microbiome of a subject. For example, biome-friendly refers to a product that may be applied to a subject that may allow the microbiome at the point of application to be maintained, minimally disrupted, and/or able to return to the microbiome after a period of time after application of the product. In embodiments, biome-friendly may refer to ammonia oxidizing microorganism-friendly, e.g. ammonia oxidizing bacteria- friendly in that the product may allow for minimal disruption of the ammonia oxidizing bacteria of a subject. In embodiments, “biome-friendly” may be referred to as “biome-compatible.”

A “natural product” is or may comprise a product that may be at least partially derived from nature. It may be anything or comprise anything produced by a living organism, and may include organisms themselves. Natural products may include or comprise an entire organism, and part of an organism (e.g., a leaf of a plant), an extract from an organism, an organic compound from an organism, a purified organic compound from an organism. Natural products may be or comprise organic substances found and cells, including primary metabolites (amino acids, carbohydrates, and nucleic acids) and secondary metabolites (organic compounds found in a limited range of species, e.g., polyketides, fatty acids, terpenoids, steroids, phenylpropanoids, alkaloids, specialized amino acids and peptides, specialized carbohydrates). Natural products may be or comprise polymeric organic materials such as cellulose, lignin, and proteins.

As used herein, “presence” or “level” may refer to a qualitative or quantitative amount of a component, e.g., any one or more of an ammonia oxidizing microorganisms, ammonia, ammonium ions, urea, nitrite, or nitric oxide. The presence or level may include a zero value or a lack of presence of a component.

As used herein, the term “surfactant”, includes compounds that may lower the surface tension, or interfacial tension, between two liquids or between a liquid and a solid. Surfactants may act as detergents, wetting agents, emulsifiers, foaming agents, and dispersants. Surfactants may include one or more of the following, alone, or in combination with those listed, or other surfactants or surfactant-like compounds: cocamidopropyl betaine (ColaTeric COAB), polyethylene sorbitol ester (e.g., Tween 80), ethoxylated lauryl alcohol (RhodaSurf 6 NAT), sodium laureth sulfate/lauryl glucoside/cocamidopropyl betaine (Plantapon 611 L UP), sodium laureth sulfate (e.g., RhodaPex ESB 70 NAT), alkyl polyglucoside (e.g., Plantaren 2000 N UP), sodium laureth sulfate (Plantaren 200), Dr. Bronner’s Castile soap, Dr. Bronner’s baby soap, Lauramine oxide (ColaLux Lo), sodium dodecyl sulfate (SDS), polysulfonate alkyl polyglucoside (PolySufanate 160 P), sodium lauryl sulfate (Stepanol-WA Extra K), and combinations thereof. Dr. Bronner’s Castile soap and baby soap comprises water, organic coconut oil, potassium hydroxide, organic olive oil, organic fair deal hemp oil, organic jojoba oil, citric acid, and tocopherol. Surfactants may include Sodium Laurylglucosides Hydroxypropylsulfonate (Suga®nate 160NC), lauramidopropyl betaine (Cola®Teric LMB); Cocamidopropyl hydroxysultaine (Cola®Teric CBS); disodium cocoamphodiacetate (Cola®Teric CDCX-LV); sodium laurylglucosides hydroxypropyl phosphate (Suga®Fax D12). Surfactants may include sodium lauroyl methyl isethionate (Iselux® LQ-CLR-SB); sodium methyl cocoyl taurate (Pureact WS Conc.); Aqua (and) Sodium Lauroyl Methyl Isethionate (and) Cocamidopropyl Betaine (and) Sodium Cocoyl Isethionate (and) Sodium Methyl Oleoyl Taurate (Iselux ®SFS-SB). Other surfactants are contemplated by this disclosure.

As used herein, the term “shelf-stable” may refer to a preparation, composition, or formulation having a suitable efficacy after a period of storage at room temperature. The period of storage may be greater than about 4 weeks. The period of storage at room temperature may be greater than about 6 months, greater than about 1 year, greater than about 2 years, or greater than about 5 years. Room temperature may include average daily temperatures between about 20° C. -25° C. In particular embodiments, the “shelf-stable” preparation, composition, or formulation may have a suitable efficacy after a period of storage of at least about 6 months at refrigeration temperature. The period of storage at refrigeration temperature may be greater than about 1 year, greater than about 3 years, greater than about 5 years, greater than about 7 years, greater than about 10 years, or greater than about 12 years. Refrigeration temperature may include average daily temperatures between about 2° C. - 6° C., or about 4° C. Other conditions for the period of storage may include, for example, an average daily relative humidity of less than about 80% and/or an average daily atmospheric pressure, or between about 12 - 15 psi.

A “unit” of a finished product refers to a single entity of finished preparation that may form an individual or complete component for use by end user or for sale. In some embodiments a unit may be a single entity but may form an individual component of a larger or more complex whole. In an embodiment, a unit may be an individual end use container that contains a preparation, for sale or for use by an end user.

“End user,” as that term is used herein, refers to a person who will use a finished preparation, e.g., by applying the finished preparation to himself or herself or which applies, or provides, the finished preparation, to a subject, e.g., another person, or an animal, e.g., a companion animal.

“End use container,” as that term is used herein, refers to a vessel that houses a preparation, e.g., finished preparation. The end use container may allow for delivery of the finished preparation from the vessel to the outside environment. In certain embodiments, the end use container may prevent or reduce retrograde flow of the contents of the container. In other embodiments, the end use container may not prevent or reduce retrograde flow of the contents of the container. The end use container may be configured to provide one-way flow and/or zero-dead volume. The end use container may not be configured to provide one-way flow and/or zero-dead volume.

The end-use container may be comprised of any suitable material that is compatible with the contents of the container and the external environment. For example, the end-use container may be made of glass, aluminum, or one or more polymers, for example, a high density polyethylene polymer.

### Preparations, Compositions, Formulations, and Products Comprising Ammonia Oxidizing Microorganisms

The present disclosure provides, inter alia, compositions comprising ammonia oxidizing microorganisms, preparations, e.g., purified and/or optimized preparations, comprising AOM, formulations comprising AOM, and various products comprising AOM, e.g., a natural product, a non-natural product, a fortified natural product, a consumer product, a therapeutic product, or a cosmetic product. The terms preparation, composition, formulation, and product may be used interchangeably herein.

Any embodiment, preparation, composition, formulation, or product of ammonia oxidizing microorganisms discussed herein may comprise, consist essentially of, or consist of (optionally axenic) ammonia oxidizing microorganisms, e.g., live ammonia oxidizing microorganisms.

The preparation may comprise or be supplemented with a product or byproduct of an ammonia oxidizing microorganism, e.g., nitrite, nitrate, nitric oxide, CoQ8. In at least some embodiments, the preparation may comprise or be supplemented with a composition that promotes growth or metabolism of ammonia oxidizing microorganisms, promotes production of products or byproducts of ammonia oxidizing microorganisms, promotes urease activity, or has a synergistic effect with ammonia oxidizing microorganisms, e.g., ammonia, ammonium salts, urea, and urease. For instance, the preparation may be supplemented with one or more of NO, nitrite, nitrate, CoQ8, ammonia, ammonium salts, urea, and urease. The supplement may be comprised in the same formulation as the ammonia oxidizing microorganisms or in a separate formulation for concurrent or combination administration. The supplement formulation may be prepared for delivery via any delivery mode, for example inhaled forms of NO, nitrite, or nitrate. The preparation may comprise, inter alia, at least one of ammonia, ammonium salts, and urea. The preparation may comprise or be supplemented with an anti-inflammatory agent or a composition that provides an anti-inflammatory effect.

The present disclosure provides for preparations comprising ammonia oxidizing microorganisms for cosmetic use.

The present disclosure provides for preparations comprising ammonia oxidizing microorganisms for therapeutic use.

In some embodiments, a preparation of ammonia oxidizing microorganisms may comprise a concentration or amount, e.g., an effective amount, of ammonia oxidizing microorganisms sufficient to have a desired cosmetic effect. The preparation may be formulated and/or delivered to impart the desired cosmetic effect locally and/or systemically.

In some embodiments, a preparation of ammonia oxidizing microorganisms may comprise a concentration or amount, e.g., an effective amount, of ammonia oxidizing microorganisms sufficient to have a desired therapeutic effect, e.g., to at least partially treat a condition or disease. The preparation may be formulated and/or delivered to impart the desired therapeutic effect locally and/or systemically.

In some embodiments, a preparation of ammonia oxidizing microorganisms may comprise a concentration or amount, e.g., an effective amount, of ammonia oxidizing microorganisms sufficient to alter, e.g., reduce or increase, an amount, concentration or proportion of a bacterium, or genus of bacteria in a subject. The bacteria may be non-pathogenic or pathogenic, or potentially pathogenic.

In some embodiments, a preparation of ammonia oxidizing microorganisms may comprise a concentration or amount, e.g., an effective amount, of ammonia oxidizing microorganisms sufficient to modulate a microbiome associated with a subject.

In some embodiments, a preparation of ammonia oxidizing microorganisms may comprise a concentration or amount, e.g., an effective amount, of ammonia oxidizing microorganisms sufficient to deliver NO to a subject. A preparation of ammonia oxidizing microorganisms may comprise a concentration or amount, e.g., an effective amount, of ammonia oxidizing microorganisms such that when administered, the preparation modulates, changes, or alters a level of nitrite or NO at a target tissue or in circulation. For instance, a preparation of ammonia oxidizing microorganisms may comprise a concentration or amount, e.g., an effective amount, of ammonia oxidizing microorganisms such that when administered, the preparation results in an increased level of nitrite or NO at a target tissue or in circulation.

The present disclosure provides, inter alia, non-limiting compositions comprising ammonia oxidizing microorganisms, e.g., N. eutropha, e.g., a purified preparation of an optimized N. eutropha. In some embodiments, the N.eutropha in the compositions has at least one property selected from an optimized growth rate, an optimized NH₄⁺ oxidation rate, and an optimized resistance to NH₄⁺.

In some aspects, the present disclosure provides compositions with a defined number of species. A composition may include only one type of species, e.g., one type of ammonia oxidizing microorganism. This disclosure also provides a composition having, e.g., N. eutropha and one other type of organism, and no other types of organism. In other examples, the composition has, e.g., N. eutropha and 2, 3, 4, 5, 6, 7, 8, 9, or 10 other types of organism, and no other types of organism. The other type of organism in this composition may be, for instance, a bacterium, such as an ammonia-oxidizing bacterium. Suitable ammonia-oxidizing microorganisms for this purpose include those in the genera Nitrosomonas, Nitrosococcus, Nitrosospira, Nitrosocystis, Nitrosolobus, or Nitrosovibrio. Likewise, the composition may also include AOA.

In some embodiments, the composition comprising, e.g., N. eutropha provides conditions that support N. eutropha viability. For instance, the composition may promote N. eutropha growth and metabolism or may promote a dormant state (e.g., freezing) from which viable N. eutropha can be recovered. When the composition promotes growth or metabolism, it may contain water and/or nutrients that N. eutropha consumes, e.g., as ammonium, ammonia, urea, oxygen, carbon dioxide, or trace minerals. In some embodiments, the composition comprising ammonia oxidizing microorganisms provides conditions that support ammonia oxidizing microorganisms viability. For instance, the composition may promote ammonia oxidizing microorganisms growth and metabolism or may promote a dormant state (e.g., freezing) or storage state as described herein, from which viable ammonia oxidizing microorganisms can be recovered. When the composition promotes growth or metabolism, it may contain water and/or nutrients that ammonia oxidizing microorganisms consumes, e.g., as ammonium ions, ammonia, urea, oxygen, carbon dioxide, or trace minerals.

In some embodiments, one or more other organisms, for example, organisms besides ammonia oxidizing microorganisms may be included in the preparation of ammonia oxidizing microorganisms. For example, a community of organisms or an organism of the genus selected from the group consisting of Lactobacillus, Streptococcus, Bifidobacter, and combinations thereof, may be provided in the preparation of ammonia oxidizing microorganisms. In some embodiments, the preparation may be substantially free of other organisms.

Preparations of ammonia oxidizing microorganisms may comprise between about between about 10³ to about 10¹⁴ CFU/ml. In some embodiments, the preparation of ammonia oxidizing microorganisms may comprise at least about or greater than about 10³, 10⁴, 10⁵, 10⁶, 10⁷, 10⁸, 10⁹, 10¹⁰, 10¹¹, 2 x 10¹¹, 5 x 10¹¹, 10¹², 2 x 10¹², 5 x 10¹², 10¹³, 2 x 10¹³, 5 x 10¹³, or 10¹⁴; or about 10³-10⁴, 10⁴-10⁵, 10⁶-10⁷, 10⁷-10⁸, 10⁸-10⁹, 10⁹-10¹⁰, 10¹⁰-10¹¹, 10¹¹-10¹², 10¹²-10¹³, or 10¹³-10¹⁴ CFU/ml.

In some embodiments, a preparation of ammonia oxidizing microorganisms may comprise between about 1 x 10⁹ to about 10 x 10⁹ CFU/ml. In some embodiments, an administered dose of the preparation may comprise about 3 x 10¹⁰ CFU, e.g., 3 x 10¹⁰ CFU per day. In some embodiments, an administered dose of the preparation may comprise about 1 x 10⁹ to about 10 x 10⁹ CFU per day, e.g., about 1 x 10⁹ to about 10 x 10⁹ CFU per day. In some embodiments, an administered dose of the preparation may comprise about 10³, 10⁴, 10⁵, 10⁶, 10⁷, 10⁸, 10⁹, 10¹⁰, 10¹¹, 2 x 10¹¹, 5 x 10¹¹, 10¹², 2 x 10¹², 5 x 10¹², 10¹³, 2 x 10¹³, 5 x 10¹³, or 10¹⁴; or about 10³-10⁴, 10⁴-10⁵, 10⁶-10⁷, 10⁷-10⁸, 10⁸-10⁹, 10⁹-10¹⁰, 10¹⁰-10¹¹, 10¹¹-10¹², 10¹²-10¹³, or 10¹³-10¹⁴ CFU per administration or per day.

In some embodiments, an administered dose of the preparation may comprise at least about 7 x 10¹⁰ CFU, e.g., 21 x 10¹⁰ CFU per week. In some embodiments, an administered dose of the preparation may comprise about 1 x 10⁹ to about 10 x 10⁹ CFU per week, e.g., about 1 x 10⁹ to about 10 x 10⁹ CFU per week. In some embodiments, an administered dose of the preparation may comprise about or greater than about 10³, 10⁴, 10⁵, 10⁶, 10⁷, 10⁸, 10⁹, 10¹⁰, 10¹¹, 2 x 10¹¹, 5 x 10¹¹, 10¹², 2 x 10¹², 5 x 10¹², 10¹³, 2 x 10¹³, 5 x 10¹³, or 10¹⁴; or about 10³-10⁴, 10⁴-10⁵, 10⁶-10⁷, 10⁷-10⁸, 10⁸-10⁹, 10⁹-10¹⁰, 10¹⁰-10¹¹, 10¹¹-10¹², 10¹²-10¹³, or 10¹³-10¹⁴ CFU per week.

In some embodiments, an administered dose of the preparation may comprise at least about 30 x 10¹⁰ CFU, e.g., 90 x 10¹⁰ CFU per month. In some embodiments, an administered dose of the preparation may comprise about 1 x 10⁹ to about 10 x 10⁹ CFU per month, e.g., about 1 x 10⁹ to about 10 x 10⁹ CFU per month. In some embodiments, an administered dose of the preparation may comprise about or greater than about 10³, 10⁴, 10⁵, 10⁶, 10⁷, 10⁸, 10⁹, 10¹⁰, 10¹¹, 2 x 10¹¹, 5 x 10¹¹, 10¹², 2 x 10¹², 5 x 10¹², 10¹³, 2 x 10¹³, 5 x 10¹³, or 10¹⁴; or about 10³-10⁴, 10⁴-10⁵, 10⁶-10⁷, 10⁷-10⁸, 10⁸-10⁹, 10⁹-10¹⁰, 10¹⁰-10¹¹, 10¹¹-10¹², 10¹²-10¹³, or 10¹³-10¹⁴ CFU per month.

In some embodiments, the preparation of ammonia oxidizing microorganisms may comprise between about 0.1 milligrams (mg) and about 1000 mg of ammonia oxidizing microorganisms. In certain aspects, the preparation may comprise between about 50 mg and about 1000 mg of ammonia oxidizing microorganisms. The preparation may comprise between about 0.1-0.5 mg, 0.2-0.7 mg, 0.5-1.0 mg, 0.5-2 mg, 0.5-5 mg, 2.5-5 mg, 2.5-7.0 mg, 5.0-10 mg, 7.5-15 mg, 10-15 mg, 15-20 mg, 15-25 mg, 20-30 mg, 25-50 mg, 25-75 mg, 50-75 mg, 50-100 mg, 75-100 mg, 100-200 mg, 200-300 mg, 300-400 mg, 400-500 mg, 500-600 mg, 600-700 mg, 700-800 mg, 800-900 mg, 900-1000 mg, 100-250 mg, 250-500 mg, 100-500 mg, 500-750 mg, 750-1000 mg, or 500-1000 mg.

The formulations disclosed herein may be shelf-stable. In certain embodiments, the formulations may have a suitable efficacy after a period of storage at room temperature. The suitable efficacy may be for a cosmetic or therapeutic effect. The shelf-stable preparation may provide a cosmetically effective result after the period of storage. The shelf-stable preparation may provide a therapeutically effective result after the period of storage.

In certain embodiments, the shelf-stable formulation may comprise CO₂. The shelf-stable formulation may comprise at least about 400 ppm CO₂. For example, the shelf-stable formulation may comprise at least about 400 ppm, 450 ppm, 500 ppm, 550 ppm, 600 ppm, or 650 ppm CO₂. The shelf-stable formulation may be substantially free of polyphosphate. The shelf-stable formulation may comprise less than about 1 µM polyphosphate. For example, the shelf-stable formulation may comprise less than about 1 µM, 0.5 µM, 0.1 µM, 50 nM, or 10 nM polyphosphate.

Advantageously, a formulation may have a pH level that promotes AOM, e.g., N. eutropha viability, e.g., metabolic activity. Urea would hydrolyze to ammonia and would raise the pH to 7 to 8. AOB are very active at this pH range and would lower the pH to about 6 where the NH₃ converts to ammonium and is unavailable. Lower pH levels, e.g. about pH 4, are also acceptable.

The ammonia oxidizing microorganisms, e.g., N. eutropha may be combined with one or more pharmaceutically or cosmetically acceptable excipients. In some embodiments, “pharmaceutically acceptable excipient” refers to a pharmaceutically-acceptable material, composition, or vehicle, such as a liquid or solid filler, diluent, solvent, or encapsulating material. In some embodiments, each excipient is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

In some embodiments, a cosmetically acceptable excipient refers to a cosmetically acceptable material, composition, or vehicle, such as a liquid or solid filler, diluent, solvent, or encapsulating material. In some embodiments, each excipient is cosmetically acceptable in the sense of being compatible with the other ingredients of a cosmetic formulation, and suitable for use in contact with the tissue or organ of humans and animals without excessive toxicity, irritation, allergic response, immunogenicity, or other problems or complications, commensurate with a reasonable benefit/risk ratio.

While it is possible for the active ingredient, e.g., ammonia oxidizing microorganisms, e.g., N. eutropha, to be administered alone, in many embodiments it is present in a pharmaceutical formulation or composition. Accordingly, this disclosure provides a pharmaceutical formulation comprising ammonia oxidizing microorganisms, for example, N. eutropha and a pharmaceutically acceptable excipient. Pharmaceutical compositions may take the form of a pharmaceutical formulation as described below.

In accordance with one or more embodiments, a preparation of ammonia oxidizing microorganisms may be formulated in order to facilitate a desired delivery mechanism or mode of administration thereof. The formulations, e.g., pharmaceutical or cosmetic formulations described herein include those suitable for, e.g., oral, enteral (including buccal, sublingual, sublabial, and rectal), parenteral (including subcutaneous, intradermal, intramuscular, intravenous, and intraarticular), inhalation (including fine particle dusts or mists which may be generated by means of various types of metered doses, pressurized aerosols, nebulizers or insufflators, and including intranasally or via the lungs), intranasal, eye, ear, rectal, injection, urogenital, and topical (including dermal, transdermal, transmucosal, buccal, sublingual, and intraocular) administration, although the most suitable route may depend upon, for example, a condition or disorder of a recipient.

In some embodiments, the preparation of ammonia oxidizing microorganisms may comprise, consist essentially of, or consist of ammonia oxidizing microorganisms in a buffer solution comprising, consisting essentially of, or consisting of disodium phosphate and magnesium chloride, for example, 50 mM Na₂HPO₄ and 2 mM MgCl₂.

In accordance with one or more non-limiting embodiments, a preparation comprising ammonia oxidizing microorganisms may be administered to a subject, e.g., for cosmetic or therapeutic purposes, as a solution, suspension, powder, liquid, drop, spray, aerosol, mist, emulsion, foam, cream, ointment, gel, hydrogel, resin, tablet, capsule, film, suppository, enema, douche, pessary, insert, patch, e.g., transdermal patch, or implantable device, e.g., stent, catheter, vaginal ring, or intrauterine device.

Devices configured to deliver a preparation comprising live ammonia oxidizing microorganisms via a desired mode of administration or otherwise via targeted delivery are also disclosed.

In accordance with one or more embodiments, the preparation may be formulated for targeted delivery to a subject, e.g., to a target tissue, region, system, or organ of a subject. For example, the preparation may be formulated for delivery to the eye, ear, nose, urogenital system, respiratory system, or gastrointestinal system of the subject. In some embodiments, targeted delivery may be based on a condition or disorder of a subject. For instance, formulation for targeted delivery may be based on a desired local or systemic effect to be achieved, e.g., a local or systemic therapeutic or cosmetic effect. In some embodiments, a target tissue, region, system, or organ of a subject may be selected for its association with a desired local or systemic effect.

The formulations may conveniently be presented in unit dosage form and may be prepared by any of the methods known in the art of pharmacy. Typically, methods include the step of bringing the active ingredient (e.g., ammonia oxidizing microorganisms, e.g., N.eutropha) into association with a pharmaceutical carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations may be presented as discrete units such as capsules, cachets or tablets, each containing a predetermined amount of, e.g., N. eutropha; as a powder or granules; as a solution or a suspension in an aqueous liquid or a non-aqueous liquid; or as an oil-in-water liquid emulsion or a water-in-oil liquid emulsion. Formulations, e.g., solutions, aerosols, sprays, and mists, may be presented in multi-dosage form, e.g., packaged units including a predetermined number of dosages, or single dosage form, e.g., packaged units including a single dose. The active ingredient may also be presented as a bolus, electuary or paste. Various pharmaceutically acceptable carriers and their formulation are described in standard formulation treatises, e.g., Remington’s Pharmaceutical Sciences by E. W. Martin. See also Wang, Y. J. and Hanson, M. A., Journal of Parenteral Science and Technology, Technical Report No. 10, Supp. 42:2 S, 1988.

The ammonia oxidizing microorganisms, e.g., N. eutropha compositions can, for example, be administered in a form suitable for immediate release or extended release. Suitable examples of sustained-release systems include suitable polymeric materials, for example semipermeable polymer matrices in the form of shaped articles, e.g., films, or microcapsules; suitable hydrophobic materials, for example as an emulsion in an acceptable oil; or ion exchange resins. Sustained-release systems may be administered orally; rectally; parenterally; intracisternally; intravaginally; intraperitoneally; topically, for example as a powder, ointment, gel, drop or transdermal patch; bucally; or as a spray.

Preparations for administration can be suitably formulated to give controlled release of ammonia oxidizing microorganisms, e.g., N. eutropha. For example, the pharmaceutical compositions may be in the form of particles comprising one or more of biodegradable polymers, polysaccharide jellifying and/or bioadhesive polymers, or amphiphilic polymers. These compositions exhibit certain biocompatibility features which allow a controlled release of an active substance. See U.S. Pat. No. 5,700,486.

Exemplary compositions include suspensions which can contain, for example, microcrystalline cellulose for imparting bulk, alginic acid or sodium alginate as a suspending agent, methylcellulose as a viscosity enhancer, dicalcium phosphate, starch, magnesium stearate and/or lactose and/or other excipients, binders, extenders, disintegrants, diluents and lubricants, mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use. The surfactant may be a zwitterionic surfactant, a non-ionic surfactant, or an anionic surfactant.

Excipients, such as surfactants that may be used with embodiments of the present disclosure may include one or more of cocamidopropyl betaine (ColaTeric COAB), polyethylene sorbitol ester (e.g., Tween 80), ethoxylated lauryl alcohol (RhodaSurf 6 NAT), sodium laureth sulfate/lauryl glucoside/cocamidopropyl betaine (Plantapon 611 L UP), sodium laureth sulfate (e.g., RhodaPex ESB 70 NAT), alkyl polyglucoside (e.g., Plantaren 2000 N UP), sodium laureth sulfate (Plantaren 200), Dr. Bronner’s Castile soap, Dr. Bronner’s Castile baby soap, Lauramine oxide (ColaLux Lo), sodium dodecyl sulfate (SDS), polysulfonate alkyl polyglucoside (PolySufanate 160 P), sodium lauryl sulfate (Stepanol-WA Extra K), and combinations thereof. Dr. Bronner’s Castile soap and Dr. Bronner’s baby soap comprises water, organic coconut oil, potassium hydroxide, organic olive oil, organic fair deal hemp oil, organic jojoba oil, citric acid, and tocopherol.

In some embodiments, surfactants may be used with ammonia oxidizing microorganisms in amounts that allow nitrite production to occur. In some embodiments, the preparation may have less than about 0.0001 % to about 10% of surfactant. In some embodiments, the preparation may have between about 0.1 % and about 10 % surfactant. In some embodiments, the concentration of surfactant used may be between about 0.0001% and about 10%. In some embodiments, the preparation may be substantially free of surfactant.

In some embodiments, the formulation, e.g., preparation, may include other components that may enhance effectiveness of ammonia oxidizing microorganisms, delivery thereof, or enhance a treatment or indication.

In some embodiments, a chelator may be included in the preparation. A chelator may be a compound that may bind with another compound, e.g., a metal. The chelator may provide assistance in removing an unwanted compound from an environment, or may act in a protective manner to reduce or eliminate contact of a particular compound with an environment, e.g., ammonia oxidizing microorganisms, e.g. a preparation of ammonia oxidizing microorganisms, e.g., an excipient. In some embodiments, the preparation may be substantially free of chelator.

Formulations may also contain anti-oxidants, buffers, bacteriostats that prevent the growth of undesired microorganisms, solutes, and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of a sterile liquid carrier, for example saline or water-for-injection, immediately prior to use. Extemporaneous solutions and suspensions may be prepared from powders, granules and tablets of the kind previously described. Exemplary compositions include solutions or suspensions which can contain, for example, suitable non-toxic, pharmaceutically acceptable diluents or solvents, such as mannitol, 1,3-butanediol, water, Ringer’s solution, an isotonic sodium chloride solution, or other suitable dispersing or wetting and suspending agents, including synthetic mono- or diglycerides, and fatty acids, including oleic acid, or Cremaphor. An aqueous carrier may be, for example, an isotonic buffer solution at a pH of from about 3.0 to about 8.0, a pH of from about 3.5 to about 7.4, for example from 3.5 to 6.0, for example from 3.5 to about 5.0. Useful buffers include sodium citrate-citric acid and sodium phosphate-phosphoric acid, and sodium acetate/acetic acid buffers. The composition in some embodiments does not include oxidizing agents.

Excipients that can be included are, for instance, proteins, such as human serum albumin or plasma preparations. If desired, the pharmaceutical composition may also contain minor amounts of non-toxic auxiliary substances, such as wetting or emulsifying agents, preservatives, and pH buffering agents and the like, for example sodium acetate or sorbitan monolaurate. In some embodiments, excipients, e.g., a pharmaceutically acceptable excipient or a cosmetically acceptable excipient, may comprise an anti-adherent, binder, coat, disintegrant, filler, flavor, color, lubricant, glidant, sorbent, preservative, or sweetener. In some embodiments, the preparation may be substantially free of excipients.

In some embodiments, the preparation may be substantially free of one or more of the compounds or substances listed in the disclosure.

Exemplary compositions for spray, aerosol, or mist administration include solutions in saline, which can contain, for example, benzyl alcohol or other suitable preservatives, absorption promoters to enhance bioavailability, and/or other solubilizing or dispersing agents. Conveniently in compositions for aerosol administration the ammonia oxidizing microorganisms, e.g., N. eutropha is delivered in the form of an aerosol spray presentation from a pressurized pack or a nebulizer, with the use of a suitable propellant, e.g., dichlorodifluoro-methane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin can be formulated to contain a powder mix of the N. eutropha and a suitable powder base, for example lactose or starch. In certain embodiments, N. eutropha is administered as an aerosol from a metered dose valve, through an aerosol adapter also known as an actuator. Optionally, a stabilizer is also included, and/or porous particles for deep lung delivery are included (e.g., see U.S. Pat. No. 6,447,743).

Formulations may be presented with carriers such as cocoa butter, synthetic glyceride esters or polyethylene glycol. Such carriers are typically solid at ordinary temperatures, but liquefy and/or dissolve at body temperature to release the ammonia oxidizing bacteria, e.g., N. eutropha.

Exemplary compositions for topical administration include a topical carrier such as Plastibase (mineral oil gelled with polyethylene). In some aspects, the composition and/or excipient may be in the form of one or more of a liquid, a solid, or a gel. For example, liquid suspensions may include, but are not limited to, water, saline, phosphate-buffered saline, or an ammonia oxidizing storage buffer. Gel formulations may include, but are not limited to agar, silica, polyacrylic acid (for example Carbopol®), carboxymethyl cellulose, starch, guar gum, alginate or chitosan. In some embodiments, the formulation may be supplemented with an ammonia source including, but not limited to ammonium chloride or ammonium sulfate.

In some embodiments, an ammonia oxidizing microorganism, e.g., N. eutropha composition is formulated to improve NO penetration, e.g., into the skin or other target tissue. A gel-forming material such as KY jelly or various hair gels would present a diffusion barrier to NO loss to ambient air, and so improve the skin’s absorption of NO. The NO level in the skin will generally not greatly exceed 20 nM/L because that level activates GC and would cause local vasodilatation and oxidative destruction of excess NO.

It should be understood that in addition to the ingredients particularly mentioned above, the formulations as described herein may include other agents conventional in the art having regard to the type of formulation in question.

The formulation, e.g., preparation, e.g., composition may be provided in a container, delivery system, or delivery device, having a weight, including or not including the contents of the container, that may be less than about 50, 100, 200, 300, 400, 500, 600, 700, 800, 900, 1000, 1500, or 2000 grams.

Suitable unit dosage formulations are those containing an effective dose, as hereinbefore recited, or an appropriate fraction thereof, of ammonia oxidizing microorganisms, e.g., N. eutropha.

A therapeutically effective amount of ammonia oxidizing microorganisms, e.g., N. eutropha may be administered as a single pulse dose, as a bolus dose, or as pulse doses administered over time. Thus, in pulse doses, a bolus administration of ammonia oxidizing microorganisms, e.g., N. eutropha is provided, followed by a time period wherein ammonia oxidizing microorganisms, e.g., N. eutropha is administered to the subject, followed by a second bolus administration. In specific, non-limiting examples, pulse doses are administered during the course of a day, during the course of a week, or during the course of a month.

In some embodiments, a preparation of ammonia oxidizing microorganisms, e.g., a formulation, e.g., a composition, may be applied for a pre-determined number of days. This may be based, for example, at least in part, on the severity of the condition or disease, the response to the treatment, the dosage applied and the frequency of the dose. For example, the preparation may be applied for about 1-3, 3-5, 5-7, 7-9, 5-10, 10-14, 12-18, 12-21, 21-28, 28-35, 35-42, 42-49, 49-56, 46-63, 63-70, 70-77, 77-84, 84-91 days., for about 1 month, for about 2 months, for about 3 months. In some embodiments, the ammonia oxidizing bacteria is administered for an indefinite period of time, e.g., greater than one year, greater than 5 years, greater than 10 years, greater than 15 years, greater than 30 years, greater than 50 years, greater than 75 years. In certain aspects, the preparation may be applied for about 16 days.

In some embodiments, a preparation of ammonia oxidizing microorganisms, e.g., a formulation, e.g., a composition, may be applied a pre-determined number of times per day. This may be based, for example, at least in part, on the severity of the condition or disease, the response to the treatment, the dosage applied and the frequency of the dose. For example, the preparation may be applied 1, 2, 3, 4, 5, 6, 7, 8, 9, 10, 11, 12, 13, 14, 15, 16, 17, 18, 19, 20, 21, 22, 23, 24 times per day.

In some embodiments, the preparation may be applied one time per day. In other embodiments, the preparation may be applied two times per day. In some embodiments, the preparation may be applied a first pre-determined amount for a certain number of days, and a second pre-determined amount for a certain subsequent number of days. In some embodiments, the preparation may be applied for about 16 days.

In accordance with one or more embodiments, the preparation may generally be compatible with a physiological environment associated with the subject. In at least some embodiments, compositions are formulated to have a substantially neutral pH or a physiological pH, for instance a pH that normally prevails in the target site for intended delivery, administration, or desired effect. Compositions may be formulated to have a pH between about 5.5 and about 8.5. Compositions may be formulated to comprise compatible conditions, e.g., pH, tonicity, with the target site of physiological environment associated with the subject.

The preparation may be formulated for transmucosal delivery and/or circulation, e.g. locally or systemically. In some embodiments, the preparation may be formulated such that ammonia oxidizing microorganisms, products thereof, or byproducts thereof (e.g., nitrate, nitrite, NO, or CoQ8) penetrate a deposit or target tissue at least 10%, 20%, 30%, 40%, 50%, 60%, 70%, 80%, 90%, or 100%. The preparation may be formulated such that 10%, 20%, 30%, 40%, 50%, 60%, 70%, 80%, 90%, or 100% of ammonia oxidizing microorganisms, products thereof, or byproducts thereof, penetrate a deposit or target tissue or enter circulation.

In accordance with one or more embodiments, the preparation may be in the form of a solution, suspension, emulsion, cream, ointment, gel, hydrogel, or liquid, e.g. drop, spray, aerosol, or mist, tablet, capsule, or device for administration to a subject.

In accordance with one or more embodiments, a preparation, composition, formulation, or product comprising ammonia oxidizing microorganisms may undergo quality control and/or testing while it is being made and/or upon its completion. International (PCT) Patent Application Publication No. WO2015/179669 (International (PCT) Patent Application Serial No. PCT/US2015/032017 as filed on May 21, 2015) which is hereby incorporated herein by reference in its entirety for all purposes describes various methods of preparing materials with ammonia oxidizing microorganisms and of testing such materials. For example, one or more parameters such as OD level, pH level, waste level, nutrient level, contaminant level, oxidation rate, nitrite level, protein concentration may be compared against a predetermined value to assess or evaluate a preparation comprising ammonia oxidizing microorganisms.

The present disclosure provides, inter alia, a kit comprising preparations of ammonia oxidizing microorganisms, as disclosed herein. Formulations may comprise discrete units, e.g., solid, liquid, or gas formulations of ammonia oxidizing microorganisms. Formulations, e.g., solutions, aerosols, sprays, and mists, may be presented in multi-dosage form (multiple use), e.g., packaged units including a predetermined number of dosages, or single dosage form (single use), e.g., packaged units including a single dose. Preparations of ammonia oxidizing microorganisms may be packaged in devices or containers configured to hold a volume of at least about less than 1 ml, 1 ml, 5 ml, 10 ml, 20 ml, 25 ml, 40 ml, 50 ml, 60 ml, 70 ml, 80 ml, 90 ml, 100 ml, or more than about 100 ml.

Kits may further comprise one or more device for administration of the preparation, for example, syringe, needle, catheter, enema, bulb, pipette (eye or ear dropper), and other devices for drug administration as known in the art. Kits may comprise instructions for use, for example instructions for administration of ammonia oxidizing microorganisms as disclosed herein or instructions for combination therapy including administration of ammonia oxidizing microorganisms. Kits may comprise a second or subsequent composition for administration in conjunction with an ammonia oxidizing preparation, as disclosed herein. For instance, kits may comprise a supplement or composition comprising a product or byproduct of ammonia oxidizing microorganisms, a composition that promotes growth or metabolism of ammonia oxidizing microorganisms, a composition that promotes production of products or byproducts of ammonia oxidizing microorganisms, a composition that promotes urease activity, or a composition that has a synergistic effect with ammonia oxidizing microorganisms, or a composition or pharmaceutical agent that treats, e.g., is approved to treat or commonly used to treat, a relevant disease, disorder, or a symptom of a relevant disease or disorder, for example an anti-inflammatory composition. Kits may comprise “biome-friendly” or “biome-compatible” products as disclosed herein, for example one or more microbiome-compatible cosmetic products. Any of the products contained in the kit may be specifically formulated to treat a target indication and/or formulated for a desired mode of delivery, as described herein.

### Natural Products; Consumer Products

In some specific embodiments, a preparation comprising ammonia oxidizing microorganisms as discussed herein may be a natural product or a consumer product. In other embodiments, a preparation of ammonia oxidizing microorganism may instead be used in conjunction with a natural product or consumer product.

Ammonia oxidizing microorganisms, e.g., N. eutropha may be associated with a variety of natural products, and examples of such products are set out below. These natural products may be comprised of formulations, compositions, or preparations disclosed throughout this disclosure.

Natural products may be or comprise products for commercial purposes, and may refer to cosmetics, dietary supplements, and foods, e.g., food, food supplements, medical food, food additive, nutraceutical, or drink, produced from natural sources. Natural products may have pharmacological or biological activity that may be of therapeutic benefit, e.g., in treating disease or conditions. Natural products may be included in traditional medicines, treatments for cosmetological purposes, and spa treatments. A natural product referred to herein may comprise any one or more of the components described as a natural product to be incorporated into a preparation or formulation comprising one or more other components, e.g., excipients. The preparation or formulation referred to as a natural product may comprise a natural product defined herein and one or more additional components or ingredients. Any of the compositions, preparations, or formulations discussed throughout this disclosure may be or comprise one or more natural products.

In some embodiments, the natural product or the fortified natural product may comprise at least one of mud, water, food-derived products, plant-derived products, extracts, and oils. The natural product or the fortified natural product may be used in a spa treatment. In some embodiments, the natural product or the fortified natural product may be incorporated into at least one of a powder, cream, lotion, wrap, scrub, eye mask, facial mask, body mask, aerosol, e.g., mist, spray, salve, wipe, stick, bandage, or soak.

In some embodiments, the natural product or fortified natural product may be provided as, or may be disposed in at least one of a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

Ammonia oxidizing microorganisms, e.g., N. eutropha may be associated with a variety of consumer products, and examples of such products are set out below and be comprised of formulations, compositions, or preparations disclosed throughout this disclosure. In some embodiments, the ammonia oxidizing bacteria, e.g., N. eutropha associated with a product is admixed with the product, for example, spread evenly throughout the product, and in some embodiments, ammonia oxidizing bacteria, e.g., the N. eutropha associated with a product is layered on the product.

In some embodiments, the preparation may be disposed in, or provided as, a powder, cosmetic, cream, stick, aerosol, e.g., mist, salve, wipe, or bandage.

In some embodiments, ammonia oxidizing bacteria, e.g., N. eutropha is associated with a powder. Powders are typically small particulate solids that are not attached to each other and that can flow freely when tilted. Exemplary powders for consumer use include talcum powder and some cosmetics (e.g., powder foundation).

In some embodiments, the ammonia oxidizing bacteria is associated with a cosmetic. The cosmetic may be a substance for topical application intended to alter a person’s appearance, e.g., a liquid foundation, a powder foundation, blush, or lipstick, and may be referred to as a preparation. The cosmetic may be any substance recited in the Food and Drug Administration regulations, e.g., under 21 C.F.R.§ 720.4.

In some embodiments, ammonia oxidizing bacteria, e.g., N. eutropha is associated with a cosmetic. The cosmetic may be a substance for topical application intended to alter a person’s appearance, e.g., a liquid foundation, a powder foundation, blush, or lipstick. Other components may be added to these cosmetic preparations as selected by one skilled in the art of cosmetic formulation such as, for example, water, mineral oil, coloring agent, perfume, aloe, glycerin, sodium chloride, sodium bicarbonate, pH buffers, UV blocking agents, silicone oil, natural oils, vitamin E, herbal concentrates, lactic acid, citric acid, talc, clay, calcium carbonate, magnesium carbonate, zinc oxide, starch, urea, and erythorbic acid, or any other excipient known by one of skill in the art, including those disclosed herein.

The preparation, e.g., the cosmetic, may be at least one of a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

In some embodiments, the formulations, compositions, or preparations described herein, may comprise, be provided as, or disposed in at least one of a baby product, e.g., a baby shampoo, a baby lotion, a baby oil, a baby powder, a baby cream; a bath preparation, e.g., a bath oil, a tablet, a salt, a bubble bath, a bath capsule; a powder (dusting and talcum), a sachet; hair preparations, e.g., hair conditioners, rinses, shampoos, tonics, face powders, cuticle softeners, nail creams and lotions, oral hygiene products, mouthwashes, bath soaps, douches, feminine hygiene deodorants; shaving preparations, e.g., aftershave lotions, skin care preparations, e.g., cleansing, face and neck, body and hand, foot powders and sprays, moisturizing, night preparations, paste masks, skin fresheners; and suntan preparations, e.g., gels, creams, and liquids.

In some embodiments, ammonia oxidizing microorganisms, e.g., the N. eutropha is associated with an aerosol, spray, or mist and these terms may be used interchangeably. An aerosol is typically a colloid of fine solid particles or fine liquid droplets, in a gas such as air. Aerosols may be created by placing the N. eutropha (and optionally carriers) in a vessel under pressure, and then opening a valve to release the contents. The container may be designed to only exert levels of pressure that are compatible with N. eutropha viability. For instance, the high pressure may be exerted for only a short time, and/or the pressure may be low enough not to impair viability. Examples of consumer uses of aerosols include for sunscreen, deodorant, perfume, hairspray, and insect repellant. The aerosol may be referred to as a spray or mist.

The compositions comprising ammonia oxidizing microorganisms, e.g., N. eutropha may also comprise one or more of a moisturizing agent, deodorizing agent, scent, colorant, insect repellant, cleansing agent, or UV-blocking agent.

In some embodiments, ammonia oxidizing microorganisms, e.g., N. eutropha are associated with cloth, yarn, or thread. Articles of clothing such as, for example, shoes, shoe inserts, pajamas, sneakers, belts, hats, shirts, underwear, athletic garments, helmets, towels, gloves, socks, bandages, and the like, may also be treated with ammonia oxidizing bacteria, e.g., N.eutropha. Bedding, including sheets, pillows, pillow cases, and blankets may also be treated with ammonia oxidizing bacteria, e.g., N.eutropha. In some embodiments, areas of skin that cannot be washed for a period of time may also be contacted with ammonia oxidizing bacteria, e.g., N.eutropha. For example, skin enclosed in orthopedic casts which immobilize injured limbs during the healing process, and areas in proximity to injuries that must be kept dry for proper healing such as stitched wounds may benefit from contact with the ammonia oxidizing bacteria, e.g., N.eutropha.

In some aspects, the present disclosure provides a wearable article comprising ammonia oxidizing microorganisms as described herein. A wearable article may be a light article that can be closely associated with a user’s body, in a way that does not impede ambulation. Examples of wearable articles include a wristwatch, wristband, headband, hair elastic, hair nets, shower caps, hats, hairpieces, and jewelry. The wearable article comprising an ammonia oxidizing bacteria, e.g., N.eutropha strain described herein may provide, e.g., at a concentration that provides one or more of a treatment or prevention of a skin disorder, a treatment or prevention of a disease or condition associated with low nitrite levels, a treatment or prevention of body odor, a treatment to supply nitric oxide to a subject, or a treatment to inhibit microbial growth.

In some embodiments, the ammonia oxidizing microorganisms, e.g., N.eutropha are associated with a product intended to contact the hair, for example, a brush, comb, shampoo, conditioner, headband, hair elastic, hair nets, shower caps, hats, and hairpieces. Nitric oxide formed on the hair, away from the skin surface, may be captured in a hat, scarf or face mask and directed into inhaled air.

Articles contacting the surface of a human subject, such as a diaper, may be associated with ammonia oxidizing microorganisms, e.g., N.eutropha. Because diapers are designed to hold and contain urine and feces produced by incontinent individuals, the urea in urine and feces can be hydrolyzed by skin and fecal bacteria to form free ammonia which is irritating and may cause diaper rash. Incorporation of bacteria that metabolize urea into nitrite or nitrate, such as ammonia oxidizing bacteria, e.g., N.eutropha, may avoid the release of free ammonia and may release nitrite and ultimately NO which may aid in the maintenance of healthy skin for both children and incontinent adults. The release of nitric oxide in diapers may also have anti-microbial effects on disease causing organisms present in human feces. This effect may continue even after disposable diapers are disposed of as waste and may reduce the incidence of transmission of disease through contact with soiled disposable diapers.

In some embodiments, the product comprising ammonia oxidizing microorganisms, e.g., N. eutropha is packaged. The packaging may serve to compact the product or protect it from damage, dirt, or degradation. The packaging may comprise, e.g., plastic, paper, cardboard, or wood. In some embodiments the packaging is impermeable to bacteria. In some embodiments, the packaging is permeable to oxygen and/or carbon dioxide.

### Shelf-Stable Ammonia Oxidizing Microorganism Preparations

The preparations disclosed herein may be shelf-stable. The shelf-stable preparations may exhibit a suitable metabolic activity of the AOM and/or Th1, Th2, Th17, or Treg inhibition activity of the AOM, even with a diminished viability of the AOM, e.g., a viability of less than 70%.

The shelf-stable preparations may comprise at least about 10² CFU/mL, 10³ CFU/mL, 10⁴ CFU/mL, 10⁵ CFU/mL, 10⁶ CFU/mL, or 10⁷ CFU/mL of AOM. The shelf-stable preparations may comprise less than about 10⁸ CFU/mL, 10⁷ CFU/mL, 10⁶ CFU/mL, 10⁵ CFU/mL, 10⁴ CFU/mL, or 10³ CFU/mL of AOM. For instance, the shelf-stable preparation may comprise between about 10² CFU/mL - 10³ CFU/mL, 10² CFU/mL - 10⁴ CFU/mL, or 10³ CFU/mL - 10⁵ CFU/mL of AOM. In particular, the shelf-stable preparation may comprise at least about 500 mg, at least about 750 mg, or between 750-1000 mg of AOM per dose.

The preparations disclosed herein may comprise 10³ cells/mL, 10⁴ cells/mL, 10⁵ cells/mL, or 10⁶ cells/mL, or more.

The shelf-stable preparation may have less than about 70%, 60%, 50%, 40%, 30%, 20%, 10%, 5%, 1%, 0.5%, or 0.1% of the AOM in a viable state. In certain embodiments, the shelf-stable preparation may have at least about 30%, 40%, 50%, 60%, 70%, 80%, 90%, 95%, 99%, 99.5%, or 99.9% of the AOM in an inactive state. The inactive AOM may be sterilized, dead, or otherwise not viable. In particular embodiments, the inactive AOM may be heat killed. For instance, the target percentage of AOM in the shelf-stable preparation may be heat killed. In certain embodiments, the AOM may be treated by freeze/thaw, ethanol, and/or aging. For instance, the target percentage of AOM in the shelf-stable preparation maybe killed by one or more of heat treatment, freeze treatment, ethanol treatment, and/or aging.

The shelf-stable preparation may comprise at least one preservative listed in Annex VI. In particular, the shelf-stable preparation may comprise at least 500 ppb of the at least one preservative. The shelf-stable preparation may comprise more than 500 ppb, more than 550 ppb, more than 600 ppb, more than 650 ppb, more than 700 ppb, more than 750 ppb, more than 800 ppb, more than 850 ppb, more than 900 ppb, more than 950 ppb, or more than 1000 ppb of the preservative. The shelf-stable preparation may comprise between about 500 ppb - 1000 ppb, between about 750 ppb - 1000 ppb, or between about 850 ppb - 1000 ppb of the preservative.

In some embodiments, the preparation if exposed to challenge with a pathogenic microorganism, will not support growth of the pathogenic microorganism. For instance, in certain embodiments, the preparation if exposed to challenge with a population of a pathogenic microorganism, will sterilize at least 50%, 60%, 70%, 80%, 90%, 95%, or 99% of the population of the microorganism. The pathogenic microorganism may be, e.g., a disease-causing microorganism.

In some embodiments, the shelf-stable preparation may be sterilized. For example, the shelf-stable preparation may be sterilized by heat or ultraviolet light.

The shelf-stable formulations (e.g., preparations or compositions) may conveniently be presented in unit dosage form and may be prepared by any of the methods known in the art of pharmacy or cosmetology. Typically, methods include the step of bringing the active ingredient (e.g., ammonia oxidizing microorganism) into association with a pharmaceutical carrier which constitutes one or more accessory ingredients. In general, the pharmaceutic or cosmetic formulations are prepared by uniformly and intimately bringing into association the active ingredient with liquid carriers or finely divided solid carriers or both and then, if necessary, shaping the product into the desired formulation.

Shelf-stable formulations may be presented as discrete units, each containing a predetermined amount of the active ingredient as a solution or suspension in an aqueous or nonaqueous liquid, as a powder or granules, or as an oil-in-water or water-in-oil liquid emulsion. Various pharmaceutically acceptable carriers and their formulation are described in standard formulation treatises, e.g., Remington’s Pharmaceutical Sciences by E.W. Martin. See also Wang, Y.J. and Hanson, M.A., Journal of Parenteral Science and Technology, Technical Report No. 10, Supp. 42:2 S, 1988; Aulton, M. and Taylor, K., Aulton’s Pharmaceutics: The Design and Manufacture of Medicines, 5^{th} Edition, 2017; Antoine, A., Gupta M.R., and Stagner, W.C., Integrated Pharmaceutics: Applied Preformulation, Product Design, and Regulatory Science, 2013; Dodou K. Exploring the Unconventional Routes - Rectal and Vaginal Dosage Formulations, The Pharmaceutical Journal, 29 Aug. 2012.

The shelf-stable preparation may be in an aqueous form. For instance, the preparation may be formulated as a liquid, e.g., spray, aerosol, or mist. Shelf-stable aqueous preparations may comprise, consist essentially of, or consist of ammonia oxidizing microorganisms in a buffer solution. The aqueous buffer solution may comprise, consist essentially of, or consist of disodium phosphate and magnesium chloride, for example, 50 mM Na₂HPO₄ and 2 mM MgCl₂.

The shelf-stable preparation may be in a substantially solid or gel-like form. For instance, the shelf-stable preparation may be formulated as a powder or gel. The preparation may be in a cream form. For instance, the shelf-stable preparation may be formulated as a cream, ointment, or lotion. The preparation may be formulated as a salve. Powder shelf-stable preparations may comprise lyophilized ammonia oxidizing microorganisms. Powder preparations may comprise talcum powder or cornstarch. Ointments may comprise anhydrous dispersions of the active agent, e.g., in a mineral oil-white petroleum base. Gels may comprise a polymer, e.g., poloxamers, xanthan gum, gellan gum, locust bean gum, and carrageenan. Gel, lotion, ointment, and salve preparations may comprise a thickener and/or emulsifier. Exemplary thickeners and emulsifiers include emulsifying wax, polysorbate 20, ceteareth 20, cellulose derivatives, guar gum, locust bean gum, xanthan gum, and gelatin. Such preparations may have a viscosity of at least about 1 mPa• s, 10 mPa• s, 100 mPa• s, 1 Pa· s, 5 Pa· s, 10 Pa· s, or 20 Pa.s at room temperature, e.g., between about 20° C. - 25° C.

Ointments, lotions, salves, and gels with greater viscosities may provide for a longer residence time than, for example, aqueous solutions. The longer residence time may further allow for a reduced dosing interval. Emulsions may comprise microspheres, microcapsules, nanoparticles, nanocapsules, micelles, liposomes, niosomes, dendrimers, or cyclodextrin complexes. Films may comprise a water-soluble polymeric film or a polyvinyl alcohol polymeric film that dissolves when in contact with bodily fluids, releasing the active agent.

Certain viscous liquid formulations may comprise a gel or gelling agent. For instance, a gelling agent may be a thermoreversible gel. A thermoreversible gel may be a liquid at lower or room temperature and turn to gel once inserted into a body cavity, e.g., intranasal, rectal, or vaginal cavity. The gel or gelling agent may allow easier administration and positioning of the dosage form. For instance, the gel or gelling agent may prevent the dosage form from leaking out of the body cavity. Thermoreversible polymers include poloxamer. Mucoadhesive polymers include sodium alginate. Gels or gelling agents may further comprise a solubility enhancer, for example, hydroxypropyl-betalcyclodextrin.

The time of onset of action for the formulations disclosed herein may be dependent on the formulation and may range from seconds to minutes to hours. The ammonia oxidizing microorganism compositions can, for example, be administered in a form suitable for immediate release or extended release. Preparations for administration can be suitably formulated to give controlled or extended release of ammonia oxidizing microorganisms. In some embodiments, controlled release formulations may be formulated as an ointment, gel, foam, or emulsion. Pharmaceutical extended release compositions may be formulated with one or more mucoadhesive agent, e.g., mucoadhesive gel or dry mucoadhesive tablet. The mucoadhesive agent may aid in attachment to a body cavity, e.g., rectal or intranasal mucosa. Similarly, solid dosage forms e.g., films, may be formulated with one or more mucoadhesive agent which may enhance dosage form positioning within the body cavity or may remain present when part of the solid dosage form melts or disintegrates. For instance, a solid dosage form may be formulated to dissolve rapidly when in contact with bodily fluid and turn to a mucoadhesive viscous solution that attaches to the body cavity wall and is gradually washed out without requiring removal.

In certain embodiments, the shelf-stable preparation may comprise CO₂. The shelf-stable preparation may comprise at least about 400 ppm CO₂. For example, the shelf-stable preparation may comprise at least about 400 ppm, 450 ppm, 500 ppm, 550 ppm, 600 ppm, or 650 ppm CO₂.

The shelf-stable preparation may be substantially free of polyphosphate. The shelf-stable preparation may comprise less than about 1 µM polyphosphate. For example, the shelf-stable preparation may comprise less than about 1 µM, 0.5 µM, 0.1 µM, 50 nM, or 10 nM polyphosphate.

The shelf-stable preparation may be formulated for oral, enteral (e.g., buccal, sublingual, sublabial, and rectal), parenteral (e.g., subcutaneous, intradermal, intramuscular, intravenous, and intraarticular), inhalation (e.g., fine particle dusts or mists which may be generated by means of various types of metered doses, pressurized aerosols, nebulizers or insufflators, and including intranasally or via the lungs), intranasal, eye, ear, rectal, injection, urogenital, or topical (e.g., dermal, transdermal, transmucosal, buccal, sublingual, and intraocular) administration.

The shelf-stable preparation may be formulated for treatment of one or more of: headaches, cardiovascular diseases, inflammation, immune responses, autoimmune disorders, liver diseases, infections, neurological diseases, psychiatric disorders, nitric oxide disorders, urea cycle disorders, congestion, vasodilation disorders, skin diseases, ophthalmic disorders, wound healing, reactions to insect bites, connective tissue disorders, and certain viral, bacterial, or fungal infections.

### Methods of Distribution of Ammonia Oxidizing Microorganism Preparations

In accordance with one or more embodiments, the methods disclosed herein may include methods of distributing the preparation or formulation, e.g., to a recipient. The recipient may be a distributor, wholesaler, retailer, or consumer, e.g., the end use consumer. The aspects disclosed herein may be performed at any one or more steps during manufacturing or distribution, for example, during manufacturing, storage, or transportation.

The shelf-stable preparations disclosed herein may be kept under non-refrigerated conditions. In particular, the preparations disclosed herein may have a suitable cosmetic and therapeutic efficacy at temperatures greater than about 4° C. Thus, the preparations disclosed herein may be stored for extended periods of time at about room temperature. Storage at room temperature is beneficial during manufacturing and distribution. For instance, the shelf-stable formulations may require less resources during manufacturing, storage, and transportation than non-shelf-stable formulations. Additionally, storage and transportation of the packaged container, e.g., end use containers having shelf-stable formulations may have environmental benefits, e.g., by reducing the use and dependence on insulated shipping materials and/or refrigerants and coolants.

The methods of distribution may include packaging the preparation comprising live ammonia oxidizing microorganisms into one or more containers, e.g., one or more end use containers. In some embodiments, during manufacturing and/or packaging, at least about 30%, 40%, 50%, 60%, 70%, 80%, 90%, 95%, 99%, 99.5%, or 99.9% of the ammonia oxidizing microorganisms are viable.

The end-use container may comprise a reservoir in which the preparation is disposed, and a dispenser through which the preparation from the reservoir can be dispensed. The reservoir may be a bottle, jar, or tube. The reservoir may be formed of plastic, paperboard, glass, aluminum, or any other suitable metal. The dispenser may be squeeze-actuated or pump-actuated. In some embodiments, the dispenser may be an orifice in the container. In some embodiments, the dispenser may be a pump. In other embodiments, the dispenser may be a valve, e.g., a pressure activated valve. In certain embodiments, the dispenser may inhibit retrograde flow of the dispensed preparation and/or atmospheric aerosols into the reservoir.

The total volume of the preparation packaged in the container may be between about 0.1 and about 100 fluid ounces, about 0.2 and about 50 fluid ounces, about 0.5 and about 25 fluid ounces, about 1.0 and about 10 fluid ounces, about 2.0 and about 7 fluid ounces, about 3 and about 5 fluid ounces. In some embodiments, the volume may be about 3.4 fluid ounces.

The container may be constructed to contain between about 0.1 and about 100 fluid ounces, about 0.2 and about 50 fluid ounces, about 0.5 and about 25 fluid ounces, about 1.0 and about 10 fluid ounces, about 2.0 and about 7 fluid ounces, or about 3 and about 5 fluid ounces. In some embodiments, the container may be constructed to contain about 3.4 fluid ounces.

The end use container may indicate one or more of the following: storage and handling of the preparation, formulation of the preparation, description of contents in the preparation, viability status of the AOM, and directions for use of the preparation. For instance, the end use container may inform the subject to store the packaged preparation at a temperature greater than about 4° C. In certain embodiments, the end use container may inform the subject to store the packaged preparation at room temperature, e.g., for a period of time greater than about 4 weeks. The end use container may inform the subject to store the packaged preparation at a temperature greater than about 4° C., e.g., about room temperature, for a period of time greater than about 1 day, 3 days, 5 days, 1 week, 2 weeks, 3 weeks, 4 weeks, 2 months, 3 months, 4 months, 5 months, 6 months, 1 year, or 5 years. In particular embodiments, the end use container may indicate an expiration date of the packaged preparation. The expiration date may be more than 6 months, more than 1 year, more than 5 years, or more than 10 years from the date of packaging or manufacture.

The end use container may inform the subject that less than about 70% of the AOM are viable. For instance, the end use container may inform the subject that less than about 70%, 60%, 50%, 40%, 30%, 20%, 10%, 5%, 1%, 0.5%, or 0.1% of the AOM in a viable state. The end use container may inform the subject that at least about 30%, 40%, 50%, 60%, 70%, 80%, 90%, 95%, 99%, 99.5%, or 99.9% of the AOM in an inactive state. The end use container may inform the subject that the packaged preparation has been sterilized.

The end use container may inform the subject to apply the preparation topically. The end use container may inform the subject to apply the preparation intranasally. The end use container may inform the subject to apply the preparation at least one of orally, enterally, intranasally, parenterally, subcutaneously, ocularly, otically, or respiratorilly.

In other embodiments, the end use container does not indicate one or more of the following: storage and handling of the preparation, formulation of the preparation, description of contents in the preparation, viability status of the AOM, and directions for use of the preparation.

The methods of distribution may include supplying (or causing a designee to supply) the preparation, e.g., packaged in a container, e.g., packaged in an end use container, to a recipient. The recipient may be an intermediary or a consumer, e.g., the end use consumer. The designee may be an entity designated by the consumer, e.g., a second end user. In some embodiments, supplying (or causing the designee to supply) the packaged end use container to the recipient may comprise making the packaged end use container available on an internet-based outlet. In other embodiments, supplying (or causing the designee to supply) the packaged end use container to the recipient may comprise making the packaged end use container available at a non-internet-based outlet, e.g., a store.

During distribution, the preparation or packaged end use container may be exposed to an environment having a temperature greater than about 4° C. For example, the preparation or packaged end use container may be exposed to an environment having a temperature greater than about 10° C. or an environment having a room temperature, e.g., between about 20° C. - 25° C.

The preparation or packaged end use container may be exposed to the environment having a temperature greater than about 4° C. for a greater than insubstantial period of time. For example, the preparation or packaged end use container may be exposed to this environment for more than a few minutes or a few hours. In certain embodiments, the preparation or packaged end use container may be exposed to this environment for a period of time of at least about 1 hour, 6 hours, 12 hours, 18 hours, 1 day, 3 days, 5 days, 1 week, 2 weeks, 3 weeks, 4 weeks, 2 months, 3 months, 4 months, 5 months, 6 months, 1 year, or 5 years. The preparation or packaged end use container may be exposed to the environment for a period of time sufficient for the preparation to reach temperature equilibrium with the environment, e.g., for the preparation to have a temperature greater than about 4° C., greater than about 10° C., greater than about room temperature (20° C. - 25° C.), or substantially the same temperature as the environment.

Thus, during distribution, the preparation or packaged end use container may attain a temperature greater than about 4° C. For example, the preparation or packaged end use container may attain a temperature greater than about 10° C. or room temperature, e.g., between about 20° C. - 25° C.

The environment having a temperature greater than about 4° C. may be a storage environment. The environment may be a shipping environment, e.g., a mail or a commercial delivery shipping environment. The mail or commercial delivery shipping environment may be a shipping environment to a consumer, e.g., the end use consumer and/or a location designated by the consumer. The environment may be a shipping environment, e.g., a cargo or a freight transport shipping environment. The cargo or freight transport shipping environment may be a shipping environment to a distributor, wholesaler, or retailer. Thus, in some embodiments, distribution of the preparation or formulation may comprise storing or transporting the packaged preparation in a non-refrigerated environment. In some embodiments, distribution of the preparation or formulation may comprise storing or transporting the packaged preparation in non-temperature-controlled packing materials. For example, in non-insulated packing materials and/or without a refrigerant or coolant.

In some embodiments, after distribution, less than about 70%, 60%, 50%, 40%, 30%, 20%, 10%, 5%, 1%, 0.5%, or 0.1% of the ammonia oxidizing microorganisms are viable. For example, in at least some embodiments, after distribution, at least about 30%, 40%, 50%, 60%, 70%, 80%, 90%, 95%, 99%, 99.5%, or 99.9% of the ammonia oxidizing microorganisms are inactive.

The methods of distribution may additionally comprise quality control of the preparation prior to packaging or quality control of the packaged preparation prior to supplying the packaged preparation to the recipient. Thus, in certain embodiments, the methods may comprise measuring at least one parameter of the preparation or the packaged preparation to determine a value. The methods may comprise comparing the value of the measured parameter to a range of pre-determined values corresponding to a pre-determined range of the parameter. The methods may comprise determining if the value is within the pre-determined range of the parameter. If the value is within the pre-determined range, the preparation or packaged preparation may be classified as accepted. If the value is outside the pre-determined range, the preparation or packaged preparation may be classified as non-accepted.

The methods may comprise distributing the accepted preparation or packaged preparation. The methods may comprise modifying the non-accepted preparation or packaged preparation to make it acceptable. The parameter may include, for example, metabolic activity of the AOM, viability of the AOM, or activated immune cell inhibition activity of the AOM, e.g., Th1, Th2, Th17, and/or Treg inhibition activity of the cell. In some embodiments, the method may include heat treating or heat killing a target percentage of the AOM to make the preparation or packaged preparation acceptable, e.g., to place the metabolic activity of the AOM, viability of the AOM, or activated immune cell inhibition activity of the AOM within the pre-determined range. For instance, the method may include heat treating or heat killing a target percentage of the AOM to place the Th1, Th2, Th17, and/or Treg inhibition activity of the AOM within the pre-determined range. The pre-determined range may be a range sufficient to treat a disease or disorder modulated by an activated immune cell, or a symptom thereof.

### Methods of Treatment With Ammonia Oxidizing Microorganisms

In accordance with one or more embodiments, a subject may be treated via administration of ammonia oxidizing microorganisms, e.g., a preparation comprising ammonia oxidizing microorganisms. As used herein, treatment of a subject may comprise administering an ammonia oxidizing microorganism composition for a cosmetic or therapeutic result. For instance, treatment may comprise treating or alleviating a condition, symptom, or side effect associated with a condition or achieving a desired cosmetic effect.

Subjects may include an animal, a mammal, a human, a non-human animal, a livestock animal, or a companion animal. The subject may be female or male. The subject may have various skin types. The subject may have various health-related profiles, including health history and/or genetic predispositions. The subject may generally have a normal microbiome, e.g., a physiological microbiome, or a disrupted microbiome. The subject may be characterized as one of the following ethnicity/race: Asian, black or African American, Hispanic or Latino, white, or multi-racial. The subject may have a Fitzpatrick Scale skin type test score of from I to IV. The subject may be of an age of less than 1, or between 1-5, 5-10, 10-20, 20-30, 30-40, 40-50, 50-60, or over 60 years.

The ammonia oxidizing microorganisms that may be used to treat a subject include all the ammonia oxidizing microorganisms, e.g., N. eutropha compositions described in this application, e.g. a purified preparation of optimized ammonia oxidizing microorganisms, for instance strain D23.

The methods may be provided to administer, or deliver a therapeutic product or a cosmetic product. The methods may comprise administering or introducing a preparation comprising live ammonia oxidizing microorganisms to a subject. The preparation may be formulated to treat a target indication and/or formulated for a desired mode of delivery.

In accordance with one or more embodiments, a preparation comprising live ammonia oxidizing microorganisms may be administered to a first tissue of a subject. The first tissue may be a deposit tissue. The first tissue may be a target tissue or a tissue other than a target tissue. The live ammonia oxidizing microorganisms, or a product thereof, e.g., nitrite and/or nitric oxide, may then move or be transported to a second tissue, e.g., via diffusion. The second tissue may be a target tissue. The target tissue may be associated with a desired local or systemic effect. The target tissue may be associated with an indication, disorder, or condition to be treated.

Ammonia oxidizing microorganism preparations may be administered, for example to the skin, for a cosmetic or therapeutic effect. For instance, administration may provide a cosmetic treatment, benefit, or effect. In some embodiments, administration may provide for treatment or improvement of one or more of oily appearance, pore appearance, radiance, blotchiness, skin tone evenness, visual smoothness, and tactile smoothness. In some embodiments, a cosmetic appearance of a subject may be altered such as may result from improved skin health. Signs of aging may be reduced, delayed, or reversed. Administration may result in a qualitative improvement in skin and/or scalp condition and/or quality. Skin smoothness, hydration, tightness, and/or softness in a subject may be improved. The present disclosure also provides a method of reducing body odor.

Administration may provide a therapeutic treatment, benefit, or effect. The present disclosure provides a method of modulating nitrite and supplying nitric oxide to a subject. The present disclosure provides various methods for the suppression, treatment, or prevention of diseases, disorders, infections, and conditions using ammonia oxidizing microorganisms. Ammonia oxidizing microorganisms may be used, for instance, to treat various diseases associated with suboptimal nitrite levels, skin diseases, and diseases caused by pathogenic bacteria.

In some embodiments, administration may provide for a reduction in inflammation. Indeed, a local or systemic anti-inflammatory effect may be demonstrated. In at least some embodiments, microbial growth may be inhibited. Skin and overall health may be improved. Inadequate circulation may be augmented. Endothelial function may be promoted. A change in level of nitrite or NO at a target tissue or in circulation may be demonstrated. In some embodiments, administration, e.g., administration of an effective amount, may modulate, change, or alter a level of nitrite or NO at a target tissue or in circulation. In some embodiments, administration, e.g., administration of an effective amount, may result in a change in the level of nitrite or NO at a target tissue or in circulation.

Administration of the compositions disclosed herein may provide transmucosal delivery and/or circulation, e.g. locally or systemically. In some embodiments, administration may provide that ammonia oxidizing microorganisms, products thereof, or byproducts thereof (e.g., nitrate, nitrite, NO, or CoQ8) penetrate a deposit or target tissue at least 10%, 20%, 30%, 40%, 50%, 60%, 70%, 80%, 90%, or 100%. In at least some embodiments, 10%, 20%, 30%, 40%, 50%, 60%, 70%, 80%, 90%, or 100% of ammonia oxidizing microorganisms, products thereof, or byproducts thereof, penetrate a deposit or target tissue or enter circulation upon administration of the compositions disclosed herein.

The preparations and methods of the present disclosure may provide for reducing an amount of undesirable microorganisms from an environment associated with a subject. The ammonia oxidizing microorganisms described herein may out-compete other organisms by, e.g., consuming scarce nutrients, or generating byproducts that are harmful to other organisms, e.g., changing a pH level that is not conducive to the undesirable organism’s growth.

The present disclosure also provides a method of promoting wound healing, including of chronic wounds, such as in a patient that has an impaired healing ability, e.g., a diabetic patient. A bandage including ammonia oxidizing microorganisms may optionally be applied to the wound.

It is appreciated that many modern degenerative diseases may be caused by a lack of NO species, and that AOM may be administered to supply those species, directly to a target tissue or via diffusion to a target tissue. Application of AOM may resolve long standing medical conditions. In certain embodiments, AOM are applied to a subject to offset modern bathing practices, especially with anionic detergents which remove AOM from the external skin.

In accordance with one or more embodiments, AOM convert ammonia to nitrite, an antimicrobial compound, and nitric oxide, a well-documented signaling molecule in the inflammatory process.

The present disclosure provides, inter alia, a method of modulating a composition of a microbiome, e.g., modulating or changing the proportions of a microbiome in an environment, e.g., a surface, e.g., a surface of a subject. This may, in turn, exhibit a health-related benefit. The method may comprise administering a preparation comprising ammonia oxidizing microorganisms to a subject. In some embodiments, the amount and frequency of administration, e.g., application, may be sufficient to reduce a proportion of pathogenic microorganisms.

Application of ammonia oxidizing microorganisms to a subject, e.g., a human subject may lead to unexpected changes in the microbiome. It may lead to increases in the proportion of normal commensal non-pathogenic species and reductions in the proportion of potentially pathogenic, pathogenic, or disease causing organisms.

An increase in the proportion of non-pathogenic bacteria may occur with a pre-determined period of time, e.g., in less than 1 day, 2 days, 3 days, 4 days, 5 days, 1 week, 2 weeks, 3 weeks, or 4 weeks, or in less than 1-3, 3-5, 5-7, 7-9, 5-10, 10-14, 12-18, 12-21, 21-28, 28-35, 35-42, 42-49, 49-56, 46-63, 63-70, 70-77, 77-84, 84-91 days.

A decrease in the proportion of pathogenic bacteria may occur with a pre-determined period of time, e.g., in less than 1 day, 2 days, 3 days, 4 days, 5 days, 1 week, 2 weeks, 3 weeks, or 4 weeks, or in less than 1-3, 3-5, 5-7, 7-9, 5-10, 10-14, 12-18, 12-21, 21-28, 28-35, 35-42, 42-49, 49-56, 46-63, 63-70, 70-77, 77-84, 84-91 days.

In accordance with one or more embodiments, a subject may be evaluated for need of treatment. In some embodiments, a subject may be selected on the basis of the subject being in need of a treatment. The present disclosure may further provide obtaining a sample from a subject and analyzing the sample.

In accordance with one or more embodiments, administration may be performed before, during, or subsequent to occurrence of a health-related condition, or in response to a warning sign, trigger, or symptom thereof. In accordance with one or more embodiments, a second amount of the preparation may be administered to the subject, e.g., a second dose.

In certain aspects, the present disclosure provides combination therapies comprising ammonia oxidizing microorganisms, e.g., a N. eutropha and a second treatment, e.g. a therapeutic. For instance, the disclosure provides physical admixtures of the two (or more) therapies are physically admixed. In other embodiments, the two (or more) therapies are administered in combination as separate formulation. The second therapy may be, e.g., a pharmaceutical agent, surgery, diagnostic, or any other medical approach that treats, e.g., is approved to treat or commonly used to treat, the relevant disease, disorder, or a symptom of the relevant disease or disorder. The second treatment may be administered before or after the administration. The effective amount can be administered concurrently with the second treatment. The second treatment may be administered via the same or a different mode of delivery. The subject may have a therapeutic level of the second treatment upon administration of the preparation. In certain embodiments, the second treatment may provide an anti-inflammatory effect or be administered to reduce inflammation at the target site. In at least some embodiments, the preparation may be administered concurrently or in conjunction with a product or byproduct of the ammonia oxidizing microorganisms, e.g., nitrite, nitrate, nitric oxide, CoQ8. In at least some embodiments, the preparation may be administered concurrently or in conjunction with a composition that promotes growth or metabolism of ammonia oxidizing microorganisms, promotes production of products or byproducts of ammonia oxidizing microorganisms, promotes urease activity, or has a synergistic effect with ammonia oxidizing microorganisms, e.g., ammonia, ammonium salts, urea, and urease.

The preparation may be administered with a microbiome cleansing preparation, for example a local or systemic antibiotic. The preparation may be administered after administration of a cleansing preparation or a bowel cleanse. The preparations may be administered pre- or post-surgical procedure, diagnostic procedure, or natural event, e.g., giving birth. The preparations may be administered before, during, or after deposit of an implantable or invasive device.

In accordance with one or more embodiments, the preparation may be administered as an analgesic or prophylactic. The preparation may be self-administered. The administration of the preparation may be device-assisted.

In some embodiments, the ammonia oxidizing microorganisms, e.g., a preparation of ammonia oxidizing microorganisms, are administered at a dose of about or greater than about 10³ - 10⁴ CFU, 10⁴ - 10⁵ CFU, 10⁵ - 10⁶ CFU, 10⁶ - 10⁷ CFU, 10⁷ - 10⁸ CFU, 10⁸ - 10⁹ CFU, 10⁹ - 10¹⁰ CFU, 10¹⁰ - 10¹¹ CFU, 10¹¹-10¹² CFU, 10¹²-10¹³ CFU, or 10¹³-10¹⁴ CFU per application, per day, per week, or per month. In some embodiments, the ammonia oxidizing microorganisms are administered at a dose of about 10⁹-10¹⁰ CFU, e.g., about 1 x 10⁹ - 5 x 10⁹, 1 x 10⁹ - 3 x 10⁹, or 1 x 10⁹ - 10 x 10⁹ CFU per application or per day. In some embodiments, the ammonia oxidizing microorganisms are administered in a volume of about less than 0.2, less than 0.5, less than 1, 1-2, 2-5, 5-10, 10-15, 12-18, 15-20, 20-25, or 25-50 ml per dose. In some embodiments, the solution is at a concentration of about 10³-10⁴, 10⁴-10⁵, 10⁵-10⁶, 10⁶-10⁷, 10⁷-10⁸, 10⁸-10⁹, 10⁹-10¹⁰, or 10¹⁰-10¹¹ CFU/ml. In some embodiments, the ammonia oxidizing microorganisms are administered as two 15 ml doses per day, where each dose is at a concentration of 10³ CFU/ml. In some embodiments, the ammonia oxidizing microorganisms are administered once, twice, three, or four times per day. In some embodiments, the ammonia oxidizing microorganisms is administered once, twice, three, four, five, or six times per week. In some embodiments, the ammonia oxidizing microorganisms is administered shortly after bathing. In some embodiments, the ammonia oxidizing microorganisms is administered shortly before sleep.

In some embodiments, the ammonia oxidizing microorganisms are administered for about 1-3, 3-5, 5-7, 7-9, 5-10, 10-14, 12-18, 12-21, 21-28, 28-35, 35-42, 42-49, 49-56, 46-63, 63-70, 70-77, 77-84, 84-91 days, e.g., for about 1 month, for about 2 months, for about 3 months. In some embodiments, the ammonia oxidizing microorganisms is administered for an indefinite period of time, e.g., greater than one year, greater than 5 years, greater than 10 years, greater than 15 years, greater than 30 years, greater than 50 years, greater than 75 years. Methods of treating a disease modulated by an activated immune cell are disclosed herein. For instance, the ammonia oxidizing microorganism compositions can, for example, be administered in a form suitable to provide treatment to a disease modulated by an activated immune cell. Methods of treating a disease modulated by an activated T helper cell or regulatory T cell are disclosed herein, e.g., T helper type 1 (Th1), T helper type 2 (Th2), T helper type 17 (Th17), or regulatory T cell (Treg).

In certain embodiments, the methods may comprise controlling a response characterized by upregulation, activation, downregulation, or suppression of an immune-related cytokine. Exemplary cytokines which may be upregulated, activated, downregulated, or suppressed by administration of the ammonia oxidizing microorganism compositions disclosed herein include IL-5, IL-13, IL-4, IFNy, IL-12, IL-2, IL-18, IL-17, IL-21, IL-22, IL-10, and TFG-B.

Methods of treating Autosomal dominant hyper-IgE syndrome (AD-HIES) are disclosed herein. For instance, the ammonia oxidizing microorganism compositions can, for example, be administered in a form suitable to provide treatment to Autosomal dominant hyper-IgE syndrome (AD-HIES).

Methods of treating immunodysregulation polyendocrinopathy enteropathy-X-linked (IPEX) are disclosed herein. For instance, the ammonia oxidizing microorganism compositions can, for example, be administered in a form suitable to provide treatment to immunodysregulation polyendocrinopathy enteropathy-X-linked (IPEX).

Exemplary diseases or disorders modulated by Th2 cells which may be treated by the methods disclosed herein include allergic diseases, e.g., atopic dermatitis, asthma, allergic rhinitis, and itch. In certain embodiments, treatment of a Th2 mediated disease or disorder may comprise upregulating or activating one or more Th2 associated cytokine, e.g., IL-5, IL-13, and

IL-4. For example, the methods may comprise administering a therapeutically effective amount of the ammonia oxidizing microorganism preparation to modulate a Th2 associated cytokine.

Exemplary diseases or disorders modulated by Th1 cells which may be treated by the methods disclosed herein include autoimmune diseases and tissue damage associated with chronic infections, e.g., Celiac disease, multiple sclerosis, and diabetes, e.g., type 1 diabetes. In certain embodiments, treatment of a Th1 mediated disease or disorder may comprise upregulating or activating one or more Th1 associated cytokine, e.g., IFNy, IL-12, IL-2, and IL-18. For example, the methods may comprise administering a therapeutically effective amount of the ammonia oxidizing microorganism preparation to modulate a Th1 associated cytokine.

Exemplary diseases or disorders modulated by Th17 cells which may be treated by the methods disclosed herein include autoimmune diseases, e.g., autoimmune inflammatory diseases, e.g., Autosomal dominant hyper-IgE syndrome (AD-HIES), rheumatoid arthritis, and irritable bowel syndrome. In certain embodiments, treatment of a Th17 mediated disease or disorder may comprise upregulating or activating one or more Th17 associated cytokine, e.g., IL-17, IL-21, and IL-22. For example, the methods may comprise administering a therapeutically effective amount of the ammonia oxidizing microorganism preparation to modulate a Th17 associated cytokine.

Exemplary diseases or disorders modulated by Treg cells which may be treated by the methods disclosed herein include those associated with regulation of allergic and autoimmune diseases, e.g., immunodysregulation polyendocrinopathy enteropathy-X-linked (IPEX). In certain embodiments, treatment of a Treg mediated disease or disorder may comprise upregulating or activating one or more Treg associated cytokine, e.g., IL-10 and TFG-B. For example, the methods may comprise administering a therapeutically effective amount of the ammonia oxidizing microorganism preparation to modulate a Treg associated cytokine.

The methods may also comprise administering a therapeutically effective amount of the ammonia oxidizing microorganism preparation to modulate a disease or disorder associated with expression of MHC II. Exemplary diseases or disorders associated with expression of MHC II include, for example, autoimmune diseases and allergic inflammation.

The methods may also comprise administering a therapeutically effective amount of the ammonia oxidizing microorganism preparation to modulate a disease or disorder associated with expression of CD86. Exemplary diseases or disorders associated with expression of CD86 include, for example, allergic inflammation.

The ammonia oxidizing microorganism compositions can, for example, be administered in form suitable to provide various local therapeutic treatment or systemic therapeutic treatment. For instance, administration may provide for treatment or improvement of a local effect. Suitable examples of local conditions that may be treated with compositions disclosed herein include local infection, inflammation, and symptoms associated therewith. Localized conditions may vary widely depending on the intended deposit or target tissue. Administration may provide for treatment or improvement of a systemic effect. Examples of systemic conditions that may be treated with compositions disclosed herein include headaches, cardiovascular diseases, inflammation, immune responses and autoimmune disorders, liver diseases, infections, neurological diseases, psychiatric disorders, pulmonary diseases, nitric oxide disorders, urea cycle disorders, congestion, vasodilation disorders, skin diseases, ophthalmic disorders, bowel disorders, auditory diseases, wound healing, reactions to insect bites, connective tissue disorders, and certain viral, bacterial, and fungal infections.

For instance, systemic conditions that may be treated with compositions disclosed herein include cardiovascular diseases such as cardioprotection, heart failure, hypertension, pulmonary, hypertension, pulmonary arterial hypertension; immune responses and autoimmune disorders such as alopecia and vitiligo; liver diseases such as non-alcoholic fatty liver disease (NAFLD), non-alcoholic steatohepatitis (NASH); neurological diseases and psychological disorders such as depression, insomnia, and diabetic neuropathy; nitric oxide disorders such as erectile dysfunction; wound healing, e.g., from bed sores and nursing home care, burns, diabetic ulcers e.g., foot ulcer, venous leg ulcer, biofilm, and mouth sores; skin diseases and disorders such as hyperhydrosis, pruritus, helomas, and subtypes of helomas; ophthalmic disorders such as blepharitis, dry eye, macular degeneration, and glaucoma; bowel disorders such as gluten sensitivity, irritable/inflammatory bowel disease, Crohn’s disease, colitis, and necrotizing enterocolitis; auditory diseases such as tinnitus, reduced hearing, vertigo, pruritus, swimmer’s ear, and congenital abnormalities; and vasodilation disorders such as Renaud’s disease, thermoregulation, and migraines. Various connective tissue disorders may also be treated. Certain viral, bacterial, and fungal infections may be treated with formulations disclosed herein, including infections caused by human papillomavirus (HPV), yeast infections, tinea versicolor, tinea unguium, tinea pedis/fungus, tinea cruris, jock itch, onychomycosis, dandruff, athlete’s foot, sinusitis, Methicillin-resistant Staphylococcus aureus (MRSA), staph, otitis media, swimmer’s ear, and bacterial vaginosis. Additional systemic conditions that may be treated with compositions disclosed herein include systemic inflammation, such as eczema, e.g., adult and pediatric eczema, hives, idiopathic uriticaria, lichen planus, insect bites including allergic reactions to insect bites, e.g., mosquito and demodex folliculorum mite, reactions to poison ivy, itchiness, keratosis pilaris, laryngitis, pemphigus, psoriasis, rosacea, folliculitis and subtypes of folliculitis, hidradenitis supportiva, perioral dermatitis, lupus rash, seborrheic dermatitis, e.g., adult and infantile seborrheic dermatitis, acne, e.g., adolescent acne, adult acne, and cystic acne, diaper rash, occupational hand dermatitis, sunburn, and dermatomyositis. Additionally, compositions disclosed herein may be delivered or applied to treat certain cosmetic indications, including but not limited to, contact dermatitis, diaper odor, e.g., adult and pediatric, body odor, feminine odor, flaking, nail hardness, body odor, oily skin, razor burn, skin appearance, skit blotchiness, skin hydration, and sun spots. Compositions disclosed herein may be applied as a bug repellant or an antimicrobial agent.

The methods may comprise administering the preparation having a temperature greater than about 4° C. For example, treatment may comprise administering the preparation having a temperature between about 4° C. - about 10° C., greater than about 10° C., between about 10° C. -about 20° C., greater than about 20° C., between about 20° C. - about 25° C., greater than about 25° C., between about 25° C. - about 37° C., greater than about 37° C., between about 37° C. - about 40° C., or greater than about 40° C.

The methods may comprise administering the preparation having been exposed to a temperature as described herein for a period of time of at least 1 day, 3 days, 5 days, 1 week, 2 weeks, 3 weeks, 4 weeks, 2 months, 3 months, 4 months, 5 months, 6 months, 1 year, or 5 years.

The preparation may be shelf-stable. Thus, in some embodiments, the methods may comprise administering the preparation having less than about 70%, 60%, 50%, 40%, 30%, 20%, 10%, 5%, 1%, 0.5%, or 0.1% of the ammonia oxidizing microorganisms in a viable state. In certain embodiments, the methods may comprise administering the preparation having at least about 30%, 40%, 50%, 60%, 70%, 80%, 90%, 95%, 99%, 99.5%, or 99.9% of the ammonia oxidizing microorganisms in an inactive state.

The methods may comprise administering the preparation topically. For example, the methods may comprise administering the preparation to the body of the subject, e.g., one or more of the face, neck, scalp, limb, hand, foot, back, buttock, torso, genitals, and chest of the subject.

The methods may comprise administering the preparation intranasally. For example, the methods may comprise administering the preparation to one or more nasal tissues selected from a nasal cavity, septal wall, nasal valve, nostril, nasopharanyx, vestibular area, turbinate (e.g., inferior, middle, superior), meatus (e.g., inferior, middle, superior), concha (e.g., inferior, middle, superior), maxillary sinus, sphenoidal sinus, sphenoethmoidal recess, ethmoidal bulla, semi-lunar hiatus, nasolacrimal duct, frontonasal duct, or olfactory region of the subject.

The methods may comprise administering the preparation orally, enterally, intranasally, parenterally, subcutaneously, ocularly, otically, or respiratorily. For example, the methods may comprise administering the preparation orally, enterally, topically, ocularly, via the auditory system, via the urogenital system, via the respiratory system, or via injection.

### Administration of Ammonia Oxidizing Microorganisms for the Treatment of Skin

In accordance with one or more embodiments, the preparations and methods disclosed herein may be used for the treatment of skin in a subject. In accordance with one or more embodiments, the preparations and methods disclosed herein may be used to reduce an appearance or effect of aging in a subject. In accordance with one or more embodiments, the preparations and methods disclosed herein may be used to improve skin integrity or condition in a subject. In accordance with one or more embodiments, the preparations and methods disclosed herein may be used to modulate skin integrity or condition in a subject. In accordance with one or more embodiments, the preparations and methods disclosed herein may be used to improve skin surface topology in a subject.

An effective amount of a preparation comprising ammonia oxidizing microorganisms may be administered to a subject, thereby treating skin, reducing an appearance or effect of aging, improving skin integrity or condition, modulating skin integrity or condition, or improving skin surface topology. The preparation may be administered in accordance with the various modes disclosed herein, e.g., topically.

The subject may have skin showing signs of aging. The preparations and methods disclosed herein may be used for conditioning skin of a subject showing signs of aging. The preparations and methods disclosed herein may be used for preventing, limiting, or inhibiting progression of an appearance or effect of aging in a subject showing signs of aging. An effective amount of a preparation comprising ammonia oxidizing microorganisms may be administered to a subject, thereby conditioning the skin of the subject showing signs of aging. An effective amount of a preparation comprising ammonia oxidizing microorganisms may be administered to a subject, thereby preventing, limiting, or inhibiting progression of an appearance or effect of aging in a subject showing signs of aging.

As disclosed herein, modulating may include modifying, e.g., affecting or impacting, skin integrity or condition. Conditioning may refer to altering a condition of the subject, e.g., of the skin of the subject. Preventing may include slowing progression, e.g., substantially slowing progression, or suppressing. Limiting may include, e.g., reducing, narrowing, or constraining progression. Inhibiting may include, e.g., restricting or arresting, progression.

In some embodiments, it may be determined whether a subject is in need of treatment for a skin condition. A subject may be predisposed for a skin condition, e.g., based on age, race, skin type, eye color, habit, or heredity.

In accordance with one or more embodiments, an effective amount of the preparation may be administered to a face of a subject. In accordance with one or more embodiments, the preparation may be administered to the body of the subject. For example, the preparation may be applied to one or more of the more of the forehead, eye region, neck, scalp, head, shoulder, arm, hands, leg, underarm, torso, chest, feet, knee, ankle, back, buttock, or genitals of the subject.

The preparation may be administered prior to onset of a skin condition in the subject. The preparation may be administered during incidence of a skin condition in the subject. The preparation may be administered subsequent to at least partial reduction of a skin condition in the subject. The preparation may be administered in response to an insult to skin of the subject. The preparation may be administered in response to a trigger or warning sign of a skin condition, e.g., aging, habitual sleep position, habitual facial expression, weight loss, ultraviolet (UV) light exposure, smoking, dehydration, or immersion.

In some embodiments, the preparation may be administered subsequent to washing the skin of the subject. For example, the preparation may be administered 30, 60, 90, 120, 150, or 180 minutes before or after the subject cleanses or showers. The preparation may be administered about 1, 2, 3, 4, 5, 6, 7, 8, 9, 10, 11, 12, 13, 14, 15, 16, 17, 18, 19, 20, 21, 22, 23, or 24 times per day. The preparation may be administered for about 1-3, 3-5, 5-7, 7-9, 5-10, 10-14, 12-18, 12-21, 21-28, 28-35, 35-42, 42-49, 49-56, 46-63, 63-70, 70-77, 77-84, or 84-91 days. The preparation may be administered within 30, 60, 90, 120, 150, or 180 minutes of the subject waking from sleep. The preparation may be administered within 30, 60, 90, 120, 150, or 180 minutes prior to the subject sleeping. The preparation may be administered within 30, 60, 90, 120, 150, or 180 minutes of the subject eating. In at least some embodiments, administration may be device-assisted.

In some embodiments, a second amount of the preparation may be administered to the subject. In at least some embodiments, a second treatment may be administered in combination with the preparation. In accordance with one or more embodiments, various combination therapies may be applied for the treatment of skin. For example, the preparations disclosed herein may be administered for treatment in combination with moisturizer, sunscreen, wrinkle cream, retinoid, alpha-hydroxy acid, antioxidant, tretinoin, glycosaminoglycan (GAG), lactic acid, malic acid, citric acid, tartaric acid, hydroquinone, kojic acid, L-ascorbic acid, licorice extract, N-acetylglucosamine, niacinamide, soy, dermal filler or injection, e.g. hyaluronic acid or calcium hydroxylapatite, botulinum toxin, laser resurfacing procedure, ultrasound therapy, chemical peel, e.g. glycolic acid peel, trichloroacetic acid or salicylic acid, or dermabrasion procedure. In at least some embodiments, the preparation may be administered in conjunction with nitrite, nitrate, and/or NO. The preparation may be administered in conjunction with an anti-inflammatory agent. In at least some embodiments, the subject may have a therapeutic level of a second treatment. The second treatment may be implemented prior to, concurrent with, or following the treatment methods disclosed herein.

In accordance with one or more embodiments, the preparation may be administered in conjunction with a medical approach that treats, e.g., is approved to treat or is commonly used to treat a skin condition, or a symptom of a skin condition. The preparation may be administered before or after a surgical or diagnostic procedure. A second treatment may involve a surgical procedure, e.g., a cosmetic surgical procedure, e.g., a lift procedure or a plastic surgery procedure.

In accordance with one or more embodiments, an amount and/or a frequency of administration may be sufficient to promote wound healing in the subject. An amount and/or a frequency of administration may be sufficient to improve a barrier function associated with skin of the subject. An amount and/or a frequency of administration may be sufficient to treat at least one of scarring (e.g., scar relating to sunburn, bed sore, wound, inflammatory lesion, or burn), skin thickening (e.g. keloid scarring), crack, fissure, heloma, sebum secretion, skin thickening, wrinkle, sun spot, skin tag, dark patch, stretch mark, spider vein, varicose vein, age spot, cellulitis, or pore appearance in the subject. An amount and/or a frequency of administration may be sufficient to reduce blotchiness or discoloration (e.g., vitiligo or post-inflammatory hyperpigmentation) associated with skin of the subject. An amount and/or a frequency of administration may be sufficient to reduce freckles associated with skin of the subject. An amount and/or a frequency of administration may be sufficient to reduce hives, allergic reaction, dermatitis (e.g. seborrheic dermatitis), warts, cold sores, candidiasis, or carbuncle associated with skin of the subject. An amount and/or a frequency of administration may be sufficient to promote firmness, hydration, elasticity, radiance, tone evenness, visual smoothness, or tactile smoothness associated with skin of the subject.

In accordance with one or more embodiments, administering an effective amount of the preparation may change or alter a level of nitrite or NO in the subject. Administering an effective amount of the preparation may modulate a microbiome associated with the skin of the subject.

In accordance with one or more embodiments, an amount and/or a frequency of administration may be sufficient to decrease an appearance, e.g., severity, of wrinkle in the subject. An amount and/or a frequency of administration may be sufficient to decrease a width of wrinkle in the subject. An amount and/or a frequency of administration may be sufficient to decrease a depth of wrinkle in the subject. An amount and/or a frequency of administration may be sufficient to decrease the length of wrinkle in the subject. At least one wrinkle treated in the subject may be a fine line, surface line, or deep furrow.

The subject may exhibit an improved condition subsequent to treatment, for example, as determined by a visual assessment or culture.

In accordance with one or more embodiments, any treatment of skin may be associated with, ancillary to, or result in the treatment, suppression, or prevention of various local or systemic indications, both cosmetic and therapeutic.

In accordance with one or more embodiments, preparations, devices, and/or kits as disclosed herein may be provided for the treatment of skin in a subject. These preparations, devices, and/or kits may be used in conjunction with the methods of treating skin as disclosed herein.

### Use of Microbiome Compatible Products With Administration of Ammonia Oxidizing Microorganisms

Microbiome compatible products may be used in conjunction with the preparations and methods disclosed herein. Various products may be considered to be “biome-friendly” or “biome-compatible.” Examples of biome-friendly products are disclosed in International (PCT) Patent Application Publication No. WO2017/004534 (International (PCT) Patent Application Serial No. PCT/US/2016/040723 as filed on Jul. 1, 2016) which is hereby incorporated herein by reference in its entirety for all purposes. Some biome-friendly products may be cosmetic or therapeutic in nature. In accordance with one or more embodiments, biome-friendly products may be used in combination with microorganisms, e.g., non-pathogenic microorganisms, e.g., ammonia oxidizing microorganisms, which may in turn be used in the form of a preparation or composition to be applied to a subject. Ammonia oxidizing compositions disclosed herein may be administered for a cosmetic or therapeutic indication in conjunction with a biome-friendly or biome-compatible product.

In accordance with one or more embodiments, a preparation, composition, formulation or product comprising ammonia oxidizing microorganisms, e.g., for cosmetic or therapeutic use, may itself be considered biome-friendly. In other embodiments, a preparation comprising ammonia oxidizing microorganisms may be used in conjunction with a biome-friendly product. In some embodiments, a preparation comprising ammonia oxidizing microorganisms may be mixed with a biome-friendly product or otherwise administered concurrently. In other embodiments, a preparation comprising ammonia oxidizing microorganisms may be distinct or separate from a biome-friendly product although potentially used in conjunction therewith. In some embodiments, a biome-friendly product is used alone. Ammonia oxidizing microorganism composition preparations for use in conjunction with a biome-friendly product may be formulated for cosmetic or therapeutic use.

Biome-friendly or biome-compatible products may be used in conjunction with an ammonia oxidizing microorganism preparation formulated for any mode of delivery, e.g., formulated for targeted delivery to a subject, e.g., to a target tissue, region, system, or organ of a subject. For example, the ammonia oxidizing microorganism preparation to be used in conjunction with a biome-friendly product may be formulated for delivery to the eye, ear, nose, urogenital system, respiratory system, or gastrointestinal system of the subject. In some embodiments, the ammonia oxidizing microorganism composition for use with a biome-friendly product may be formulated for targeted delivery based on a condition or disorder of a subject. For instance, the formulation for targeted delivery may be based on a desired local or systemic effect to be achieved, e.g., a local or systemic therapeutic or cosmetic effect.

Biome-friendly cosmetic products that may be used with the present disclosure may be, or include, or be disposed in any one or more of a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps, e.g., foaming body cleansers, and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

Products, e.g., microbiome-compatible cosmetic products, e.g., shampoos, conditioners, and cleansers, as described herein may be used in conjunction with the treatment of a condition, disease, or disorder. These cosmetic products may be used in conjunction with administration of the ammonia oxidizing microorganisms for therapeutic or cosmetic purposes. For example, throughout the treatment period or cosmetic period of time of administering the ammonia oxidizing bacteria to a subject, the microbiome-compatible cosmetic products may be used. The microbiome-compatible cosmetic products may be used for a period of time prior to commencement of treatment of the therapeutic or cosmetic condition through administration of ammonia oxidizing bacteria to a subject. The microbiome-compatible cosmetic products may be used for a period of time subsequent to commencement of treatment of the therapeutic or cosmetic condition through administration of ammonia oxidizing bacteria to a subject. The microbiome-compatible cosmetic products may be used for a period of time subsequent to discontinuation of therapeutic or cosmetic treatment of the condition through administration of ammonia oxidizing bacteria to a subject.

In some embodiments, the subject may apply one or more cosmetic product, and wait a period of time before administration of the ammonia oxidizing microorganisms. In other embodiments, the subject may administer the ammonia oxidizing microorganisms, and wait a period of time before applying one or more cosmetic products.

The period of time the subject may wait may be about 1 minute, 5 minutes, 10, 15, 20, 25, 30, 45, 60, 90, 120 minutes, or 3 hours, 4, 5, 6, 7, 8, 12, 18, 24 hours after applying one or more cosmetic product and prior to administration of ammonia oxidizing microorganisms.

The period of time the subject may wait may be about 1 minute, 5 minutes, 10, 15, 20, 25, 30, 45, 60, 90, 120 minutes, or 3 hours, 4, 5, 6, 7, 8, 12, 18, 24 hours after administering the ammonia oxidizing microorganisms and prior to applying one or more cosmetic products.

While specific embodiments of the subject invention have been discussed, the above specification is illustrative and not restrictive. Many variations of the invention will become apparent to those skilled in the art upon review of this specification.

### Containers, E.g., End Use Container, Delivery Devices

Containers and/or delivery devices, e.g., containers, e.g., delivery devices, e.g., end use containers are provided as a housing for the preparation, e.g., a finished preparation. In some embodiments, the container, or delivery device may also serve the purpose of delivering the preparation, e.g., finished preparation, e.g., to a surface or a subject.

The container and/or delivery device may be configured to store and/or deliver any preparation or product disclosed herein. The preparation may be delivered to a site, an environment, or a surface, e.g., of a subject, with or without additional components. In certain embodiments, other components may be delivered simultaneously or consecutively, e.g., at least partially before or at least partially after, the delivery of the preparation commences. In certain embodiments, the container or delivery device may comprise or be referred to as a delivery system. In some embodiments, the delivery of one component is still occurring when the delivery of the second begins, so that there is overlap. This is sometimes referred to herein as “simultaneous” or “concomitant” or “concurrent delivery.” In other embodiments, the delivery of one component ends before the delivery of the other treatment begins. This is sometimes referred to herein as “successive” or “sequential delivery” or “consecutive delivery.”

A barrier may be provided as part of or within the container to prevent fluid communication between the interior of the container and the exterior environment. The barrier may be in the form of a valve, e.g., check valve, filtering material, film, wax, lipid, polymer, control release material, e.g., a gel, and other materials that may either provide a permanent or temporary barrier between the interior of the container and the exterior environment.

Upon actuation of the container, the barrier may be disrupted to allow disposal of the cosmetic product from the container to the exterior environment, or a site, an environment, or a surface, e.g., of a subject, to contact the cosmetic product with a site, an environment, or a surface, e.g., of a subject.

The container may comprise a delivery system. The delivery system may be an applicator or be configured to deliver the contents of the preparation.

The delivery system may be configured to deliver a preparation to a surface of a subject, e.g., to a skin surface. The preparation may be in the form of a particle, or a plurality of particles having a particle size to enhance delivery or enhance positioning or contact with a desired target site. The preparation may be in the form of a liquid, solid, in a suspension or in a solution. The preparation may be in the form of a powder, cream, ointment, or lotion.

In certain embodiments, the delivery system may comprise a pump to deliver the contents of the interior of the container to a target site, e.g., an environment, e.g., a surface of a subject, e.g., skin of a subject.

In some embodiments, the container may be a single-use container. The container may or may not be pre-loaded (e.g., loaded by a manufacturer or user) with contents, e.g., preparation, and may be used once by a user, e.g., a consumer or medical professional to deliver the contents of the container to a target site, e.g., an environment, e.g., a surface of a subject, e.g., skin of a subject.

In other embodiments, the container may be a multiple-use container in which the container may or may not be pre-loaded (e.g., loaded by a manufacturer or user) with contents, e.g., preparation, and may be used one or more times by a user, e.g., a consumer or medical professional to deliver the contents of the container to a target site, e.g., an environment, e.g., a surface of a subject, e.g., skin of a subject. The container may be re-loaded (e.g., loaded by a manufacturer or user) with contents e.g., preparation comprising ammonia oxidizing microorganisms, and may be used again by a same or different user, e.g., a consumer or medical professional to deliver the contents of the container to a target site, e.g., an environment, e.g., a surface of a subject, e.g., skin of a subject.

Pre-loading or re-loading of the contents, e.g., preparation may comprise a sterilization process to ensure that the contents of the container are sterilized.

In some embodiments, the container, e.g., end use container, may be in the form of a syringe, bottle, ampule, applicator, pouch, e.g., spout pouch, e.g., with screw top. A pump may be attached to the bottle in order to dispense the contents from the container. The container may provide for an aerosol spray or mist. The container may be a squeezable container, e.g., squeeze bottle or tube, to allow dispensing of the contents through an opening that is covered by a closure. The container may have a screw type closure, a non-spill closure, a snap cap closure, or a snap flap closure. The container may have an over cap that resides over the dispensing area in which dispensing of the contents occurs. The closure may be fully removable or partially removable, e.g., fully removable form the body of the container, or partially removable and attached by a hinge. The container may be a single use package, e.g., a laminated packet, e.g., that may be torn open to dispense the contents, and disposed after use.

The container, e.g., end use container may be configured to inhibit retrograde flow, e.g., backflow, e.g., reverse flow, e.g., rearward movement, of material, e.g., the preparation, into the end use container. The container, e.g., end use container may be configured to inhibit retrograde flow, e.g., backflow, e.g., reverse flow, e.g., rearward movement, of material, e.g., a contaminant, into the end use container. The contaminant may be is atmospheric, e.g., an aerosol, or a liquid, e.g., water, or solid, or a gas, e.g., oxygen.

In other embodiments, the container, e.g., end use container may not be configured to inhibit retrograde flow, e.g., backflow, e.g., reverse flow, e.g., rearward movement, of material, e.g., the cosmetic product, into the end use container. The container, e.g., end use container may not be configured to inhibit retrograde flow, e.g., backflow, e.g., reverse flow, e.g., rearward movement, of material, e.g., a contaminant, into the end use container.

The end use container may comprise a reservoir in which the preparation is disposed, and a dispenser through which the preparation from the reservoir can be dispensed. In some embodiments, the dispenser inhibits retrograde flow of material into the reservoir. In other embodiments, the dispenser does not inhibit retrograde flow of material into the reservoir.

The end use container may comprise a reservoir in which the preparation is disposed, and a dispenser through which the preparation from the reservoir can be dispensed. In some embodiments, the dispenser inhibits retrograde flow of dispensed preparation, or atmospheric aerosols, into the reservoir. In other embodiments, the dispenser does not inhibit retrograde flow of dispensed preparation, or atmospheric aerosols, into the reservoir.

In certain embodiments, the end use container may be an anti-retrograde flow dispenser comprising a first pressure activated valve disposed in the dispenser and proximal to the reservoir and a second pressure activated valve disposed in the dispenser and distal to the reservoir, wherein the activation pressure of the first valve is higher than the activation pressure of the second valve.

In certain embodiments, the end use container may comprise an anti-retrograde mechanism configured to prevent movement of the preparation in a direction opposite the operational direction associated with dispensing the product.

In some embodiments, the container may be substantially free of organisms, e.g., microorganisms. In embodiments the container may be free of other organisms. The container may be sterilized to provide for a container substantially free or free of organisms, e.g., microorganisms.

The container or preparation may be disposed in a powder, cosmetic, cream, stick, aerosol, salve, wipe, or bandage. The container or preparation may be provided as a powder, cosmetic, cream, stick, aerosol, salve, wipe, or bandage.

In some embodiments, the container may comprise an indicator component. The indicator component may a color marker that may develop a color upon the contact of a microorganisms to the interior of the container.

The container may be constructed of any material suitable for housing the contents, e.g., a cosmetic product, e.g., a finished cosmetic product disclosed herein. For example the container may be constructed and arranged to be at least partially resistant to at least one of gaseous exchange, water, and/or light. For example, the container may be constructed of a glass or polymeric material. In other embodiments, the container may allow passage of gaseous exchange, water, and/or light.

The end use container may be composed of or comprise a polymer, e.g., polyethylene terephthalate (PET), high density polyethylene (HDPE), polypropylene, polycarbonate, polytetrafluoroethylene (teflon®), polyviylidene fluoride (PVDF), or a cellulosic. The end use container may be composed of or comprise glass.

A sensor, e.g., an oxygen sensor, may be included in the end use container that may indicate a presence of viable bacteria. The end use container may allow for passage of at least about 5, 10, 20, 30, 40, 50, 60, 70, 80, 90, 95, 99, or 100 percent of transmission of ionizing radiation, e.g., with gamma rays, e.g., with x-rays, e.g., from an isotope, e.g., cobalt 60, or with ultraviolet, e.g., ultraviolet C (UVC) through the end use container.

The containers described herein may be adapted to deliver one or more cosmetic products. The containers described herein may be adapted to deliver one or more therapeutic products.

The weight of the container, delivery system, or delivery device, including or not including the contents of the container may be less than about 50, 100, 200, 300, 400, 500, 600, 700, 800, 900, 1000, 1500, or 2000 grams.

### EXAMPLES

The function and advantages of these and other embodiments can be better understood from the following examples. These examples are intended to be illustrative in nature and are not considered to be limiting the scope of the invention.

### Example 1: Production of Heat Killed Ammonia Oxidizing Microorganisms to Simulate Inactivated Ammonia Oxidizing Microorganisms

N.eutropha (D23) were heat killed to simulate inactivated D23. Briefly, live D23 were fixed in storage buffer (storage state). Secreted metabolites were washed out. The D23 in storage buffer were heat killed by exposure to 60° C. for two hours. Metabolic activity of the heat killed D23 was measured by Griess reaction after 1 hour of incubation at 37° C. in buffer with 50 mM ammonia. Live D23 were incubated for 1 hour and as a control.

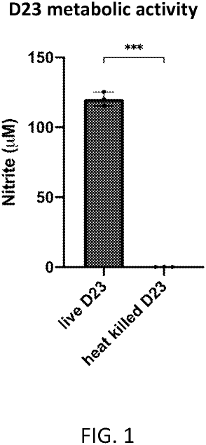

The results are shown in the graph of FIG. 1. Heat killed D23 produced no nitrite and had no measured metabolic activity.

### Example 2: Peripheral Blood Mononuclear Cells (PBMC) Treated With Live and Heat Killed Ammonia Oxidizing Microorganisms

Heat killed D23 were prepared as described in Example 1. PBMCs were treated with live and heat killed D23 to determine the treatment effect of D23 microorganism components as compared to live microorganisms. PBMCs were thawed and seeded by culturing 7x10⁶ cells/mL in RPMI 1640 cell media (distributed by Gibco™, Thermo Fisher Scientific, Waltham, MA) with 10% heat inactivated fetal bovine serum (Hi-FBS) (distributed by Thermo Fisher Scientific, Waltham, MA) and 2 mM Glutamax™ (distributed by Thermo Fisher Scientific, Waltham, MA).

After 16 hours of seeding, 3x10⁷ CFU/mL (MOI = 4.3) of live or heat killed D23 were added to the PBMC culture. After 1 hour, Th2 was stimulated with anti-CD3 antibodies and a cocktail of cytokines (Human Th2 Cell Differentiation Kit, distributed by CellxVivo™, R&D Systems, Minneapolis, MN). The cocktail did not contain anti-IFNg or anti-IL-12 antibodies.

After 3 days of Th2 stimulation, samples were collected. The samples were designated for WST-1, ELISA, and RT-qPCR/flow cytometry experiments.

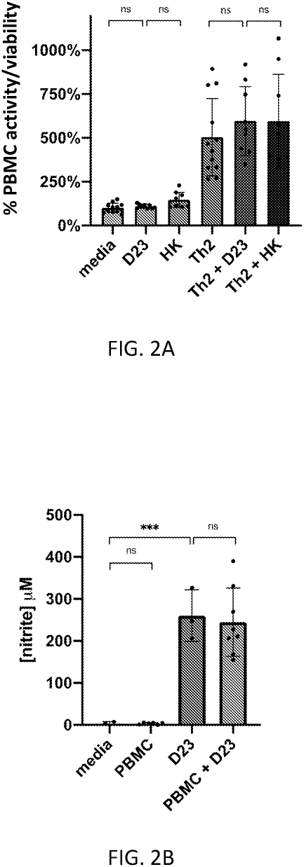

No toxicity was observed in the PBMC and D23 or heat killed D23 co-cultures. The results are shown in the graphs of FIGS. 2A-2B. FIG. 2A is a graph of percent PBMC activity and viability measured by WST-1 at 72 hours post-stimulation. FIG. 2B is a graph of nitrite levels measured by Griess reagent at 72 hours post-stimulation.

### Example 3: Th2 Inhibition by Ammonia Oxidizing Microorganisms and Heat Killed Ammonia Oxidizing Microorganisms

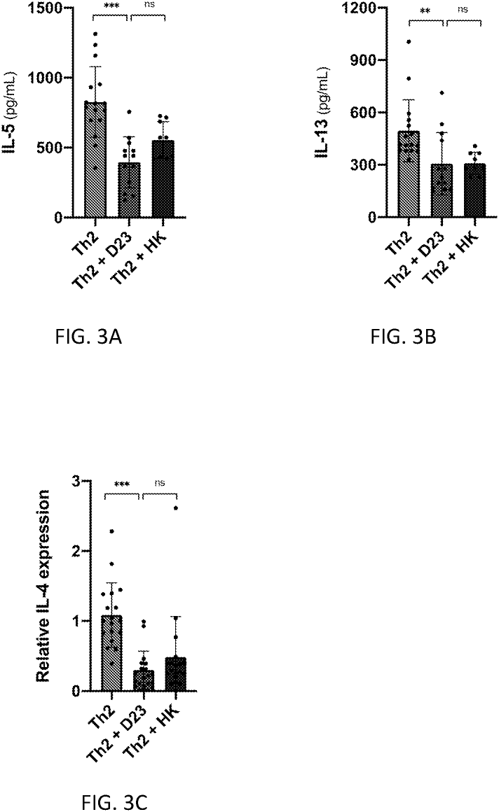

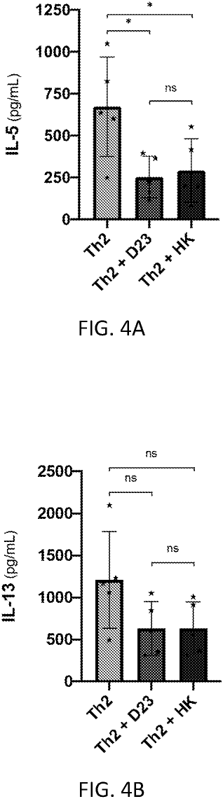

Th2 stimulated cell cultures with D23 or heat killed D23 were prepared as described in Example 2. IL-5 and IL-13 expression in treated cells from a single donor was measured at 72 hours post-stimulation by ELISA. The results are shown in the graphs of FIGS. 3A-3B. IL-4 expression in treated cells from a single donor was measured 72 hours post-stimulation by RTqPCR. Relative IL-4 expression is shown in the graph of FIG. 3C. IL-5 and IL-13 expression in treated cells from 5 donors was measured at 72 hours post-stimulation by ELISA. The average expression is shown in the graphs of FIGS. 4A-4B.

As shown in the data presented in FIGS. 3A-3C and 4A-4C, Th2 stimulated cell cultures with D23 and heat killed D23 show similar IL-5, IL-13, and IL-4 expression. Thus, heat killed D23 show similar activity with Th2 inhibition as live D23.

### Example 4: LpS, CD3/28 Stimulated Cells With Ammonia Oxidizing Microorganisms, Heat Killed Ammonia Oxidizing Microorganisms, and Ammonia

LpS and CD3/28 stimulated cells were co-cultured with D23 or heat killed D23, as previously described. The cells were also co-cultured with 6 mM NH₃ to determine the metabolic activity of microorganisms with ammonia.

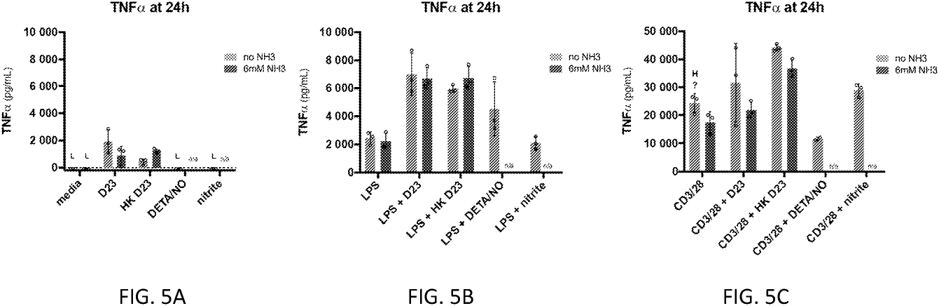

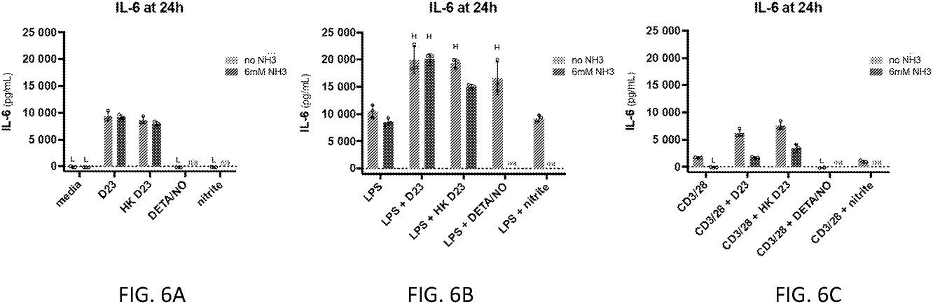

TNFα and IL-6 expression was measured 24 hours post-stimulation by CBA. The results are shown in the graphs of FIGS. 5A-5C and 6A-6C, respectively. As shown in the graphs, heat killed D23 induce an innate immune response comparable with live D23. However, no phenotype of D23 ammonia oxidation metabolism was detected in the co-cultures with 6 mM NH₃.

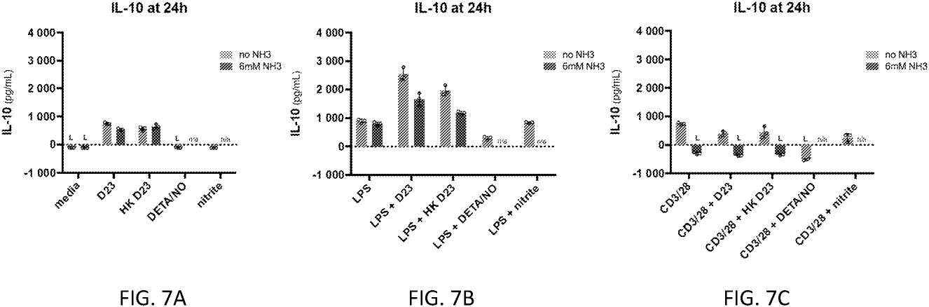

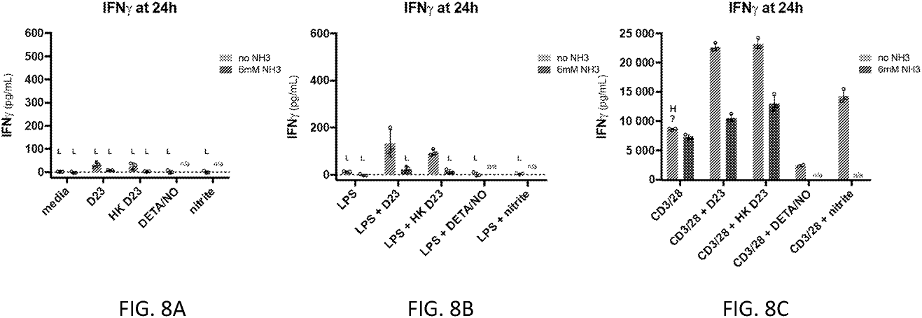

IL-10 and IFNy expression was measured 24 hours post-stimulation by CBA. The results are shown in the graphs of FIGS. 7A-7C and 8A-8C, respectively. As shown in the graphs, heat killed D23 seems to drive an adaptive immune response independently of D23 ammonia oxidation metabolism. Live D23 induces slightly more IL-10 in the presence of LpS as compared to heat killed D23 (FIG. 7B). Again, no phenotype of D23 ammonia oxidation metabolism was detected in the co-cultures with 6 mM NH₃.

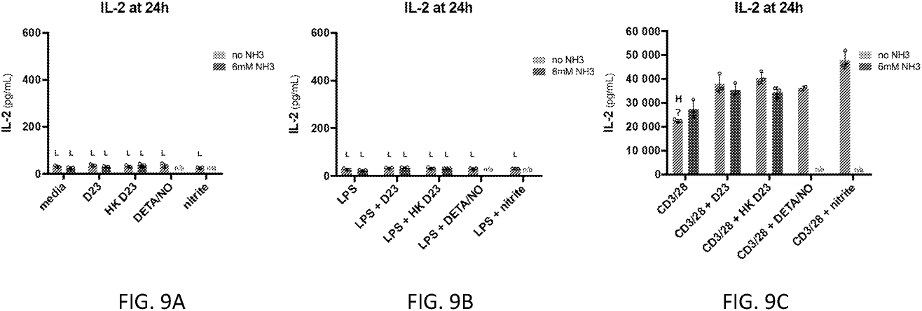

IL-2 expression was measured 24 hours post-stimulation by CBA. The results are shown in the graphs of FIGS. 9A-9C. As shown in the graphs, heat killed D23 and live D23 do not modulate IL-2, in the presence or absence of ammonia.

IL-4 and IL-17A expression was measured 24 hours post-stimulation by CBA. The data is not included because no IL-4 or IL-17A expression was detected by the assay.

As shown in the data, heat killed D23 show similar metabolic activity with the measured cytokines as live D23.

Ammonia oxidizing microorganism preparations may have a suitable efficacy (immune response) with a population of inactive ammonia oxidizing microorganisms.

### Example 5: Peripheral Blood Mononuclear Cells (PBMC) Stimulation and Treatments

PBMC were thawed, washed into Hank’s medium (ATCC) with 10% heat-inactivated fetal bovine serum (FBS) (Gibco™, Thermo Fisher Scientific, Waltham, MA) then resuspended into PBMC culture media (RPMI 1640 medium without phenol red and glutamine (Gibco™, Thermo Fisher Scientific, Waltham, MA) supplemented with 10% heat-inactivated FBS (Gibco™, Thermo Fisher Scientific, Waltham, MA) and 2 mM Glutamax (Gibco™, Thermo Fisher Scientific, Waltham, MA)). PBMC were seeded in 6 or 24 well plates (Corning, Inc., Corning, NY) and incubated at 37° C. with 5% CO₂ at a concentration of 7-8 x 10⁶ cells/mL. Live or heat killed AOB prepared as described in Example 1 were added at a multiplicity of infection (MOI) of 4 one hour prior to Th2 stimulation.

Cells were then stimulated using either the CellxVivo™ human Th2 cell differentiation kit, (R&D Systems, Minneapolis, MN) following the manufacturer’s recommendations, or 1 µg/mL Staphylococcal enterotoxin B (SEB) (Sigma Aldrich, St. Louis, MO). Neutralizing antibodies were used at 10 µg/mL and refreshed daily until sample collection. The following neutralizing antibodies were used: mouse anti-human IFNy clone K3.53 (MAB2852), mouse anti-human IL-12p70 clone 24910 (MAB219), mouse anti-human IL-10 clone 948505 (MAB9184) and the isotype controls mouse IgG2a (MAB003) and mouse IgG1 (MAB002) (all from R&D Systems, Minneapolis, MN).

TLR blocking antibodies were added to PBMC at a final concentration of 10 µg/mL one hour prior to AOB treatment and refreshed daily until sample collection. The following TLR blocking antibodies were used: TLR1, TLR2, TLR4, TLR5, TLR6 (TLR2 from R&D Systems, Minneapolis, MN, all others from Invivogen, San Diego, CA). TLR8 inhibitor compounds CU-CPT9a (Invivogen, San Diego, CA) or CU-CPT8m (MedChemExpress, Monmouth Junction, NJ) were added to PBMC 1 hour prior to AOB treatment at final concentrations between 100 nm-10 µM. TLR9 antagonist ODN TTAGGG (A151) or negative control (Invivogen, San Diego, CA) were added at a final concentration of 1 µM and refreshed daily until sample collection.

After all treatments or stimulation, plates were shaken at 600 rpm for 30 s and incubated at 37° C. with 5% CO₂. Samples were collected three days post-stimulation (unless otherwise indicated) and spun at 500 x g for 10 min. Cell-free supernatants were frozen at -20° C. for later cytokine quantification and cell pellets were processed for RNA extraction or flow cytometry analysis.

The stimulated and treated cells were used to obtain data presented in Examples 7 and 8, below.

### Example 6: Bacterial Metabolite Quantification

Nitrite and nitric oxide production were measured to determine the metabolic activity of live or heat killed AOB. Nitrite was quantified using the Griess assay. Briefly, samples were mixed 1:1 with a solution of equal volumes Griess Reagent A (1.5 N Hydrochloric Acid, 58 mM Sulfanilamide) and Griess Reagent B (0.77 mM NNEQ), incubated for 20 minutes at room temperature protected from light then quantified by measuring OD540nm using a 96 well plate spectrophotometer. Concentrations of nitrite were then calculated based on a standard curve of sodium nitrite.

Nitric oxide was quantified using the fluorescent dye DAF-2 (Abcam, Cambridge, UK). Briefly, samples were mixed 1:1 with PBMC/AOB culture media containing 5 µM DAF-2 and incubated at 37° C. with 5% CO₂ for 1 hour. A standard curve of the fluorescent molecule DAF-2T (Abcam, Cambridge, UK) was used to quantify nitric oxide production. Measurements were either taken after one hour in AOB culture media or 72 hours in PBMC culture media.

### Example 7: Th2 Inhibition by Ammonia Oxidizing Microorganisms Involves RNA Sensor Toll-Like Receptor 8 (TLR8) Signaling

Multiple types of bacterial immuno-modulatory components have been identified, from metabolites (e.g. SCFAs, indoles) to cell wall components (e.g. LPS, PSA) and nucleic acids (e.g. CpG DNA, RNA). It was shown that AOB’s ability to block Th2 polarization does not substantially rely on metabolites. Heat killed AOB appeared as effective as live AOB in Th2 mediated inhibition. Therefore, the immuno-modulatory molecule nitric oxide produced by AOB is unlikely to be involved in this phenotype.

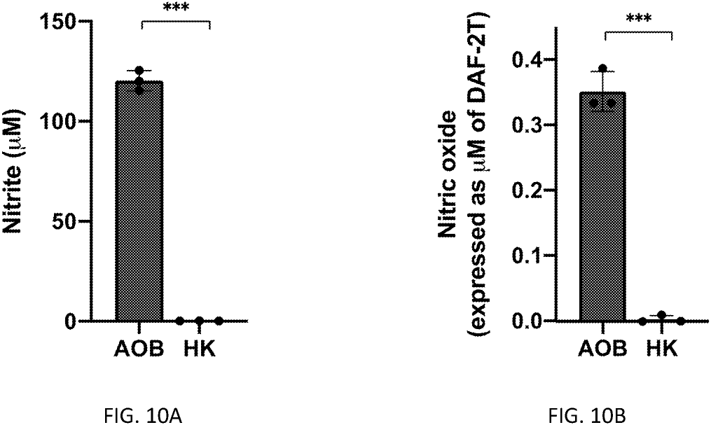

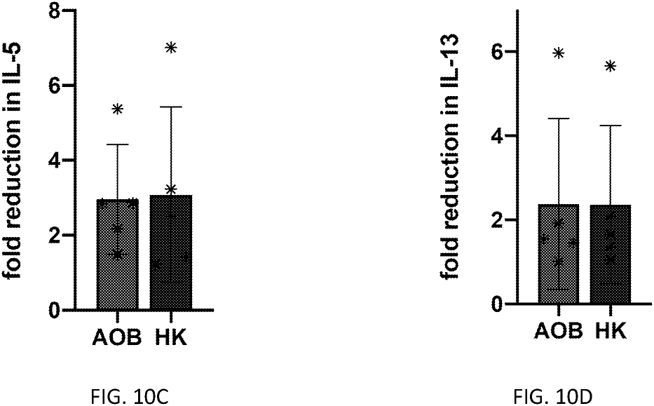

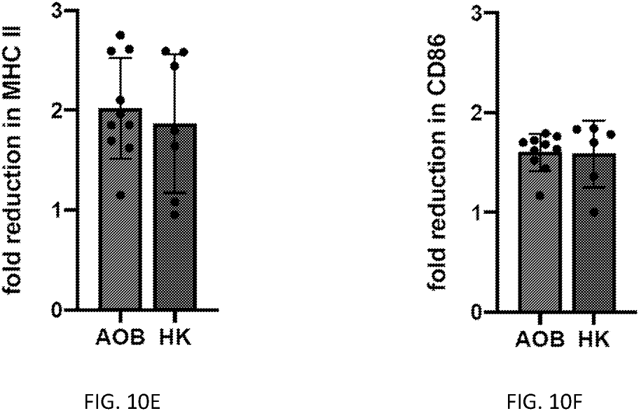

Briefly, a study was performed to identify AOB bacterial component(s) responsible for Th2 inhibition. To discriminate between secreted metabolites and cell wall/intracellular components, live AOB were compared heat killed AOB that were washed to remove any residual secreted molecules. The heat killed AOB were prepared as described in Example 1. As opposed to live AOB, heat killed AOB were unable to produce nitrite or nitric oxide indicating the lack of metabolic activity (FIGS. 10A-10B, p<0.001). Heat killed AOB were as efficient as live AOB at inhibiting the Th2 markers IL-5 and IL-13 (FIGS. 10C-10D). Heat killed AOB were as efficient as live AOB at inhibiting CD11c+ surface proteins MHC II and CD86 (FIGS. 10E-10F). These data indicate that AOB metabolic activity and associated secreted molecules, such as the immunomodulatory molecule nitric oxide, are not required for Th2 inhibition. Rather, these findings point towards a role of structural or intracellular components.

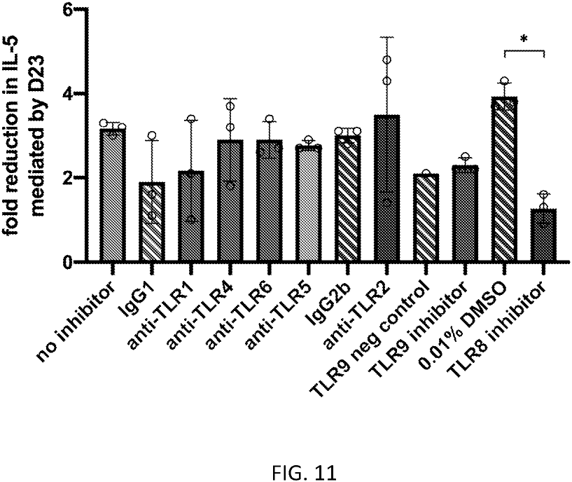

The role of toll-like receptors (TLRs) in Th2 inhibition was studied. Major TLR classes were examined by applying individual TLR inhibitors or neutralizing antibodies to PBMC prior to AOB treatment and Th2 stimulation. The impact of the TLR inhibitors or neutralizing antibodies on AOB-mediated Th2 inhibition was measured. None of TLR1/2/4/6/9 inhibitions significantly affected AOB’s ability to impact IL-5 production (FIG. 11).

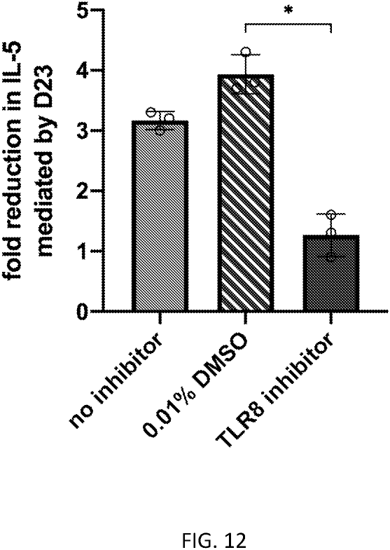

Using a collection of TLR inhibitors and blocking antibodies it was found that TLR8 signaling seems critical for AOB mediated Th2 inhibition. Inhibiting the RNA sensor TLR8 resulted in a 67.6% reduction of AOB mediated IL-5 inhibition compared to the vehicle control (FIG. 12, p<0.05).

TLR8 is known to detect single stranded RNA and is located inside the endosome. While not wishing to be bound by theory, it is believed that AOB are likely endocytosed and RNA released by cell lysis triggers TLR8 signaling. TLR8 stimulation was reported to induce IL-10 production in other models, although the exact mechanism is still unclear.

These data suggest that AOB RNA may play an important role in the early signaling cascade that leads to a suppression of Th2 responses by AOB.

### Example 8: Mechanism of Ammonia Oxidizing Microorganism Mediated Th2 Modulation

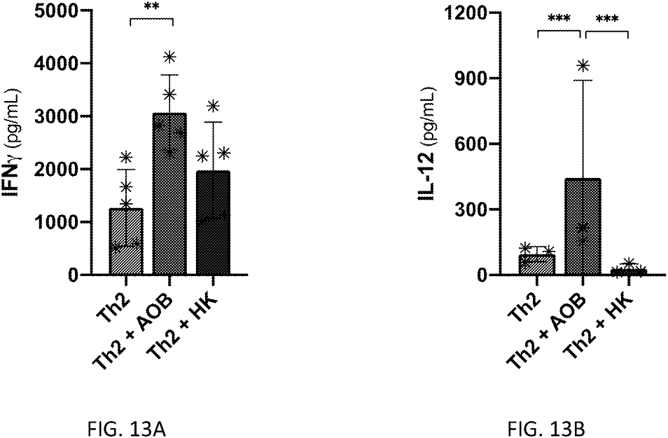

It was determined that AOB-mediated Th2 modulation does not substantially involve Th1. Neutralizing the Th1 effector IFNy or the Th1 differentiating factor IL-12 did not significantly impair the AOB’s ability to block Th2. Heat killed AOB, which blocked Th2 polarization as efficiently as live AOB, did not induce as strong a Th1 response as live AOB (FIGS. 13A-13B).

### Example 9: Therapeutic Safety of Shelf-Stable Ammonia Oxidizing Microorganism Preparations

AOB are predicted to be safe as supported by the Generally Recognized as Safe (GRAS) label issued by the FDA for D23. There are no known reports of live AOB being associated with infections or pathologies. Phase I human safety trials with live AOB reported no adverse events. There are no known reports of adverse events of live AOB when used as a cosmetic.

As shown above, heat killed AOB inhibit Th2 polarization as efficiently as live AOB. The potential use of heat killed AOB would be expected to further increase their safety profile by preventing colonization and circumventing any unexpected potential infectivity or in situ toxin production, common concerns for the use of live bacterial/biotherapeutic products.

As also shown above, heat killed AOB do not induce as strong a Th1 response as live AOB (FIGS. 13A-13B). Thus, heat killed AOB could be beneficial in the context of atopic diseases with a Th1 component (e.g. celiac disease) or atopic patients also suffering from Th1-mediated diseases (e.g. multiple sclerosis, type 1 diabetes). Thus, heat killed AOB may be used to treat a Th2 mediated disease, without exacerbating a diagnosed Th1 mediated disease.

Non-viable, inactive, and/or heat killed ammonia oxidizing bacteria N. eutropha D23 show a promising therapeutic potential on atopic diseases due to its ability to block Th2 polarization and key cytokines involved in IgE production, eosinophilia & itch, and a favorable safety profile. The mechanism of action leading to the Th2 pathway inhibition is believed to be IL-10 mediated inhibition of dendritic cell activation triggered by TLR8 sensing of AOB RNA.

### Example 10: Fold Reduction in IL-5 Mediated by D23 Cells Treated Under Different Conditions

Additional tests were performed to determine the efficacy of D23 cells treated under different conditions. Freeze/thaw treated cells, ethanol treated cells, aged cells, heat treated cells, and live cells were studied in the same IL-5 assay. Freeze/thaw (F/T) cells were treated according to the following protocol. Live D23 cells were centrifuged in 1.5 mL centrifuge tubes at 16,000 x G for 5 minutes. The supernatant was removed. 1 mL of PBS was aliquoted into each tube. 1 mL of the D23 stocks was aliquoted into 1.5 mL cryotubes. The cells underwent two freeze-thaw cycles at -80° C. and room temperature. The cells were kept at 4° C. after thawing. It is believed the cells were killed by the freeze/thaw treatment.

Ethanol (EtOH) cells were treated according to the following protocol. 5 mL of D23 stock was pipetted into two 50 mL tubes. The tubes were centrifuge at 3000 x G for 45 minutes. The supernatant was removed. 5 mL of 70% ethanol was pipetted into each tube. The tubes were placed in a running biosafety cabinet uncapped and allowed to completely dry out. Once dry, 5 mL of PBS were pipetted into each tube. Both tubes were consolidated into one stock. It is believed the cells were killed by the ethanol treatment.

Aged cells were stored at 4° C. for the duration of aging. The first set of cells (old 1) was aged approximately 27 months. The second set of cells (old 2) was aged approximately 17 months. The third set of cells (old 3) was aged approximately 12 months.

Heat killed (HK) cells were treated as previously described. Briefly, live D23 were fixed in storage buffer (storage state). Secreted metabolites were washed out. The D23 in storage buffer were heat killed by exposure to 60° C. for two hours.

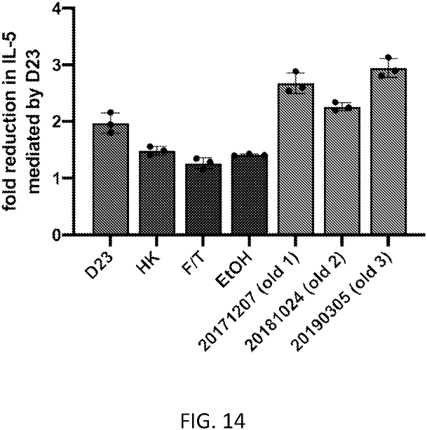

A reduction in IL-5 was observed for all cell treatments. The data is presented in the graph of FIG. 14. Accordingly, freeze/thaw cells, ethanol treated cells, and aged cells show a similar effect as live and heat killed D23 cells.

### Example 11: Treatment of Eczema by Topical Administration of Shelf-Stable Compositions Containing Ammonia Oxidizing Microorganisms

A shelf-stable N.eutropha D23 cream composition was made containing: Water (Aqua), Glycerin, Caprylic/Capric Triglyceride, Jimmondsia Chinensis (Jojoba) Seed Oil Squalane, Prunus Amygdalus Dulcis (Sweet Almond) Oil , Butyrospermum Parkii (Shea Butter) Carbomer, Polyacrylic acid, Ceteareth 20, Gyceryl Stearate, Potassium Sorbate, Citric Acid, Tocopheryl Acetate, N. eutropha, Sodium Bicarbonate, and Sodium Hyaluronate. The composition contained 7.5 x 10⁷ D23 cells/mL.

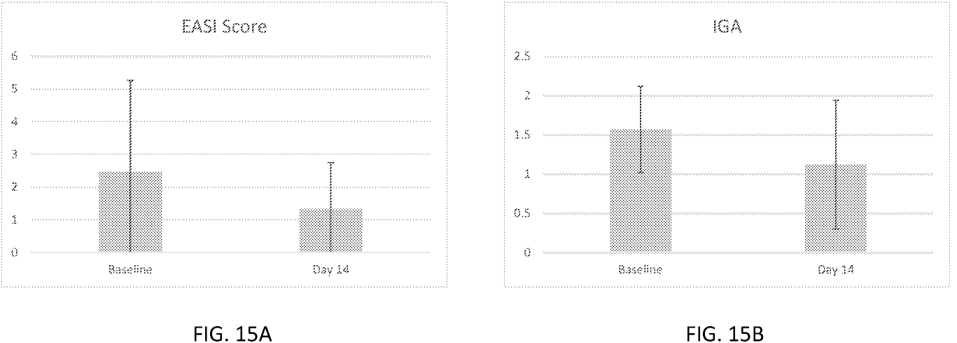

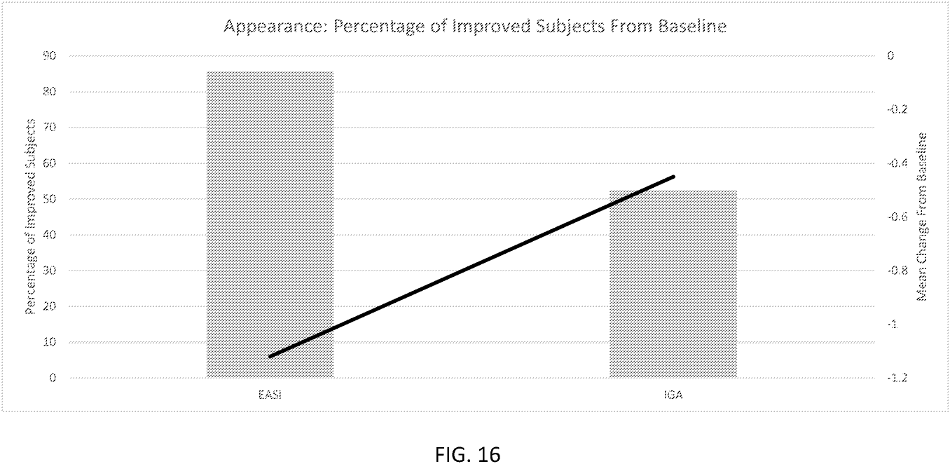

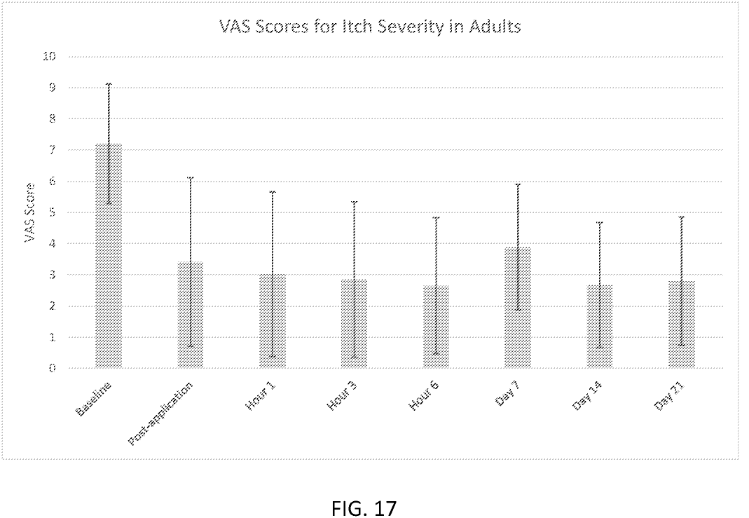

Twenty-one subjects applied the composition topically to a target tissue exhibiting symptoms of eczema twice daily and as needed for 14 days. Treatment was discontinued and a follow-up occurred at 21 days. The subjects scored a mean change in appearance of the eczema from baseline. The results are presented in the graphs of FIGS. 15A-15B and 16.

As shown in the graph of FIG. 15A, average Eczema Area and Severity Index (EASI) score decreased after 14 days. In particular, average EASI score decreased from about 2 to about 1. EASI is a validated scoring system that grades the physical signs of atopic dermatitis and eczema. Thus, administration of the compositions disclosed herein may provide for a decrease in EASI score. In some instances, administration may provide for a decrease in EASI score by at least about 5 points, for example, at least about 4 points, about 3 points, about 2 points, or about 1 point. In some instances, administration may provide for a decrease in EASI score by at least about 100%, for example, at least about 75%, at least about 50%, or at least about 25%.

As shown in the graph of FIG. 15B, average Investigator’s Global Assessment (IGA) score also decreased after 14 days. In particular, average IGA score decreased from about 1.5 to about 1. IGA is a five-point scale that provides a global clinical assessment of atopic dermatitis severity ranging from 0 to 4, where 0 indicates clear, 2 is mild, 3 is moderate, and 4 indicates severe atopic dermatitis. A decrease in score relates to an improvement in signs and symptoms. In some instances, administration may provide for a decrease in IGA score by at least about 5 points, for example, at least about 4 points, about 3 points, about 2 points, about 1 point, about 0.5 points, or about 0.25 points. In some instances, administration may provide for a decrease in IGA score by at least about 100%, for example, at least about 75%, at least about 50%, at least about 25%, at least about 20%, at least about 15%, or at least about 10%.

As shown in the graph of FIG. 16, the percentage of improved subjects from baseline was high for EASI score (slightly over 80%) and for IGA score (slightly over 50%). The mean change from baseline for EASI score was about -1.1 points, while the mean change from baseline for IGA score was about -0.5 points.

Accordingly, the shelf-stable D23 compositions disclosed herein are effective for treatment of eczema.

### Example 12: Treatment of Pruritus by Topical Administration of Shelf-Stable Compositions Containing Ammonia Oxidizing Microorganisms

Fourteen adult subjects applied the shelf-stable D23 cream composition of Example 11 to a target tissue for treatment of pruritus twice daily and as needed for 14 days. Treatment was discontinued and a follow-up occurred at 21 days. The subjects scored itch severity on a visual analogue scale (VAS) of 1-10 with 0 being no pruritus; < 3 being mild pruritus; ≥ 3 to <7 being moderate pruritus; ≥ 7 to <9 being severe pruritus; and ≥ 9 being very severe pruritus. The results are presented in the graph of FIG. 17.

As shown in the graph of FIG. 17, VAS score declined over the study period. In particular, VAS score declined from an average of about 7 (severe pruritus) at baseline to an average of slightly less than 3 (mild pruritus) at 14 and 21 days. In some instances, administration may provide for a decrease in VAS score for itch by at least about 5 points, for example, at least about 4 points, about 3 points, about 2 points, or about 1 point. In some instances, administration may provide for a decrease in VAS score for itch by at least about 100%, for example, at least about 75%, at least about 50%, or at least about 25%.

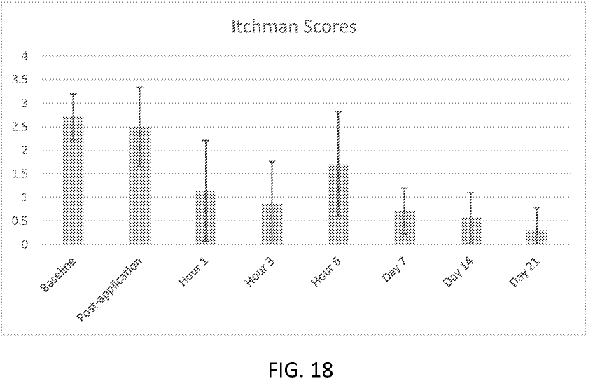

Seven pediatric subjects applied the shelf-stable D23 cream composition of Example 11 to a target tissue for treatment of pruritus twice daily and as needed for 14 days. Treatment was discontinued and a follow-up occurred at 21 days. The subjects scored itch severity on an itchman scale with 0 being comfortable; 1 being itches a little; 2 being itches more; 3 being itches a lot; and 4 being itches most terribly. The results are presented in the graph of FIG. 18.

As shown in the graph of FIG. 18, itchman score declined over the study period. In particular, itchman score declined from an average of about 2.75 (itches more to itches a lot) at baseline to an average of 0.5 to 0.25 (comfortable to itches a little) at 14 and 21 days, respectively. In some instances, administration may provide for a decrease in itchman score for by at least about 5 points, for example, at least about 4 points, about 3 points, about 2 points, or about 1 point. In some instances, administration may provide for a decrease in itchman score by at least about 100%, for example, at least about 75%, at least about 50%, or at least about 25%.

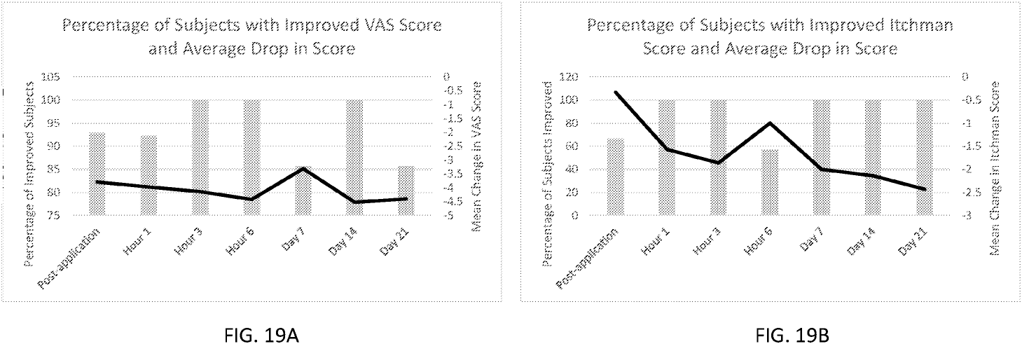

As shown in the graphs of FIGS. 19A-19B, the percentage of improved adult subjects from baseline was high for VAS score (85% - 100%) and the percentage of improved pediatric subjects from baseline was high for itchman scores (60% - 100%). The mean change from baseline of VAS score for adult subjects score was -3.5 to -4.5 points, while the mean change from baseline of itchman score for pediatric subjects was -0.5 to -2.5 points.

Accordingly, the shelf-stable D23 compositions disclosed herein are effective for treatment of pruritus.

ANNEX VI - ASEAN Cosmetic Documents ANNEX VI - Part 1 - List of preservatives allowed for use of cosmetic products

ANNEX Vl - PART 1

LIST OF PRESERVATIVES ALLOWED

| Reference Number | Substance | Maximum authorized concentration | Limitations and requirements | Conditions of use and warnings wherein must be printed on the label |
|---|---|---|---|---|
| a | b | c | d | e |
| 1 | Benzoic acid (CAS No. 66-86-01 and its sodium and (CAS No 832-321) | Rinse off products except and care products 2.5% (acid) Oral care products: 1.7% (acid) Leave on products. 0.5% (acid) | | |
| 1a | Salts of benzoic acid other than that listed under reference number 1 and esters of benzoic acid | 0.5% (acid) | | |
| | Frapicnic acid and the salts | 3% (acid) | | |
| 3 | Satcylic acid and its salts (+) | 0.5% (acid) | Not to be used in preperations for children under three years of age. except for shampoos | • Not to be used for children under three years of age⁽²⁾ |
| 4 | Sorbic acid (beta-2.4-dioxane acid) and its salts | 0.6% (acid) | | |
| 5 | Formaldehyde and paraformaldenhyde | 0.2% (except for products for oral hygiene; 0.1% (for oral hygiene) expressed to free formiatehyde | Prohibited in senses disbursers (sprays) | |
| 6 | Entry moved or deleted | | | |
| 7 | Bipheryl-2-of (a-phenytehenes and its salts | 0.2% expressed as phenol | | |
| 8 | Zinc pyrithione (+ (CAS No (3453-41-3) | Hair products, 1.0% Other products 0.5% | Rinse off products only. Not for use in products for oral hygiene | |
| 9 | inorganic sulptites and hydrogenesuphites (s) | 0.2% expressed as free SO_{2.} | | |
| 10 | Entry moved or deleted | | | |
| 11 | Chlorotetamolian | 0.5% | prohibited in aerosol dispensed (sprays) | Contains embodiments |
| 12 | 4-Hydroxybenzoic acid and its salts and esters | 0.4% (acid) for 1 esters 0.8%(acid) for mixture of esters | | |
| 13 | 3-acetyl-8-methytoyrane-2.4(1H)-doine (Dehydroacetic acid) and its salts | 0.8% (acid) | Prohibited in aerosol dispensers (sprays) | |
| 14 | Formic acid and its sodium salt | 0.8% (expressed as acid) | | |
| 15 | 3,3-D-4,4-hexamethylenediamydibenzamidine (Ciromohennazation) and its salts (including inematite) | 0.8% | | |
| 16 | Thaizmezal(INN) | 0.007% (of Mg) %muted with other material compounds authorized by this Directive the maximium concentration of Mg remains fixed at 0.007% | For eye make-up and eye make-up remover only | Contains shipment |
| 26 | A-Chloro-3, 5-xylecci | 0.5% | | |
| 27 | | 0.5% | | |
| 28 | | 0.9% | | |
| 29 | | 1.0% | | |
| 30 | | | | |
| 31 | | 0.2% | | |
| 32 | | 0.5% | | |
| 33 | | 0.5% | | |
| 34 | | 1% | | |
| 35 | | 1% | Rinse-off products For other products | |
| 36 | Entry deleted | | | |
| 37 | | 0.1% | | |
| 38 | | 0.1% | | |
| 49 | | | | |
| 50 | | 0.3% | | |
| 51 | | 0.5% | | |
| 52 | | | | |
| 53 | | 0.1% | | |
| 54 | | | | |
| 55 | | 0.15% | Only for products to be removed by rinsing | |

With regard to Annex VI:
1. Preservatives are substances which may be added to cosmetic products for the primary purpose of inhibiting the development of micro-organisms in such products.
2. The substances marked with the symbol (+) may also be added to cosmetics products in concentration other than those laid down in the ANNEX for other purposes apperent from the presentation of the products, e.g. as deodorants in soaps or as anti-dandruff agents in shampoos.
3. Other substances used in the formulation of cosmetic products may also have anti-microbial properties and thus help in the preservation of the products, as, for instance, many essential oils and some alcohols. These substances are not included in the ANNEX.
4. For the purposes of this list
   “Salts” is taken to mean: salts of the cations sodium, potassium, calcium, magnesium, ammonium, and ethanolamines, salts of the anions chloride, bromide, sulphate acetate.
   “Esters” is taken to mean: esters of methyl, ethyl, propyl, isopropyl, butyl, isobutyl, phenyl.
5. All finished products containing formaldehyde or substances in this ANNEX and which release formaldehyde must be labelled with the warning “contains formaldehyde” where the concentration of formaldehyde in the finished product exceeds 0.05%.

1. A method of distributing a preparation, comprising:
providing a preparation comprising live ammonia oxidizing microorganisms (AOM),
wherein, during distribution, the preparation is exposed to an environment having a temperature greater than about 4° C.,
thereby distributing the preparation.

2. A method of treating a subject, comprising:
administering to the subject a therapeutically effective amount of a preparation comprising ammonia oxidizing microorganisms (AOM),
wherein less than about 70%, 60%, 50%, 40%, 30%, 20%, 10%, 5%, 1%, 0.5%, or 0.1% of the ammonia oxidizing microorganisms are viable.

3. A method of treating a subject, comprising:
administering to the subject a therapeutically effective amount of a preparation comprising ammonia oxidizing microorganisms (AOM),
wherein the preparation is at a temperature greater than or about room temperature e.g., between about 20° C. - 25° C., when administered.

4. A method of treating Autosomal dominant hyper-IgE syndrome (AD-HIES) in a subject, comprising:
administering to the subject a therapeutically effective amount of a preparation comprising ammonia oxidizing microorganisms (AOM),
thereby treating the Autosomal dominant hyper-IgE syndrome (AD-HIES).

5. A method of treating immunodysregulation polyendocrinopathy enteropathy-X-linked (IPEX) in a subject, comprising:
administering to the subject a therapeutically effective amount of a preparation comprising ammonia oxidizing microorganisms (AOM),
thereby treating the immunodysregulation polyendocrinopathy enteropathy-X-linked (IPEX).

6. A method of producing a shelf-stable cosmetic, therapeutic, or consumer product, comprising:
formulating a preparation comprising ammonia oxidizing microorganisms (AOM) into a powder, cream, ointment, or lotion; and
packaging the preparation into the cosmetic, therapeutic, or consumer product,
wherein less than about 70%, 60%, 50%, 40%, 30%, 20%, 10%, 5%, 1%, 0.5%, or 0.1% of the ammonia oxidizing microorganisms in the cosmetic or therapeutic product are viable.

7. The method of any of the preceding claims, comprising providing the preparation comprising live ammonia oxidizing microorganisms.

8. The method of any of the preceding claims, wherein less than about 70%, 60%, 50%, 40%, 30%, 20%, 10%, 5%, 1%, 0.5%, or 0.1% of the ammonia oxidizing microorganisms are viable.

9. The method of any of the preceding claims, wherein, the preparation is exposed to an environment having a temperature greater than about 4° C.

10. The method of any of the preceding claims, wherein the preparation is packaged into a container.

11. The method of any of the preceding claims, wherein the preparation is packaged into a plurality of containers, e.g., at least 2, 3, 6, 8, 10, or 20 containers.

12. The method of any of the preceding claims, wherein the preparation is packaged into an end use container.

13. The method of any of the preceding claims, wherein the preparation is packaged into a plurality of separate end use containers, e.g., at least 2, 4, 6, 8, 10, 20, 50, or 100 end use containers.

14. The method of any of the preceding claims, comprising supplying (or causing a designee to supply) the preparation or packaged end use container to a recipient.

15. The method of any of the preceding claims, wherein, during distribution, the preparation or packaged end use container is exposed to an environment having a temperature greater than about 10° C.

16. The method of any of the preceding claims, wherein, during distribution, the preparation or packaged end use container is exposed to an environment having a room temperature, e.g., between about 20° C. - 25° C.

17. The method of any of the preceding claims, wherein, during distribution, the preparation or packaged end use container attains a temperature greater than about 4° C.

18. The method of any of the preceding claims, wherein, during distribution, the preparation or packaged end use container attains a temperature greater than about 10° C.

19. The method of any of the preceding claims, wherein, during distribution, the preparation or packaged end use container attains a temperature greater than about room temperature, e.g., between about 20° C. - 25° C.

20. The method of any of the preceding claims, wherein, during distribution, the preparation or packaged end use container is exposed to the environment for a period of time of at least about 1 hour, 6 hours, 12 hours, 18 hours, 1 day, 3 days, 5 days, 1 week, 2 weeks, 3 weeks, 4 weeks, 2 months, 3 months, 4 months, 5 months, 6 months, 1 year, or 5 years.

21. The method of any of the preceding claims, wherein the environment is a storage environment.

22. The method of any of the preceding claims, wherein the environment is a shipping environment, e.g., a mail or a commercial delivery shipping environment.

23. The method of any of the preceding claims, wherein the environment is a shipping environment, e.g., a cargo or a freight transport shipping environment.

24. The method of any of the preceding claims, wherein, during distribution, the preparation or packaged end use container attains a temperature greater than about 4° C. for at least about 1 hour, 6 hours, 12 hours, 18 hours, 1 day, 3 days, 5 days, 1 week, 2 weeks, 3 weeks, 4 weeks, 2 months, 3 months, 4 months, 5 months, 6 months, 1 year, or 5 years.

25. The method of any of the preceding claims, wherein, during distribution, the preparation or packaged end use container attains a temperature greater than about 10° C. for at least about 1 hour, 6 hours, 12 hours, 18 hours, 1 day, 3 days, 5 days, 1 week, 2 weeks, 3 weeks, 4 weeks, 2 months, 3 months, 4 months, 5 months, 6 months, 1 year, or 5 years.

26. The method of any of the preceding claims, wherein, during distribution, the preparation or packaged end use container attains a temperature greater than about room temperature, e.g., between about 20° C. - 25° C., for at least about 1 hour, 6 hours, 12 hours, 18 hours, 1 day, 3 days, 5 days, 1 week, 2 weeks, 3 weeks, 4 weeks, 2 months, 3 months, 4 months, 5 months, 6 months, 1 year, or 5 years.

27. The method of any of the preceding claims, wherein supplying (or causing the designee to supply) the preparation or packaged end use container to the recipient comprises making the preparation or packaged end use container available on an internet-based outlet.

28. The method of any of the preceding claims, wherein supplying (or causing the designee to supply) the preparation or packaged end use container to the recipient comprises making the preparation or packaged end use container available at a non-internet-based outlet, e.g., a store.

29. The method of any of the preceding claims, wherein, upon packaging, e.g., into an end use container, at least about 30%, 40%, 50%, 60%, 70%, 80%, 90%, 95%, 99%, 99.5%, or 99.9% of the ammonia oxidizing microorganisms are viable.

30. The method of any of the preceding claims, wherein, upon packaging, e.g., into an end use container, at least about 30%, 40%, 50%, 60%, 70%, 80%, 90%, 95%, 99%, 99.5%, or 99.9% of the ammonia oxidizing microorganisms are active.

31. The method of any of the preceding claims, wherein, after distribution, less than about 70%, 60%, 50%, 40%, 30%, 20%, 10%, 5%, 1%, 0.5%, or 0.1% of the ammonia oxidizing microorganisms are viable.

32. The method of any of the preceding claims, wherein, after distribution, at least about 30%, 40%, 50%, 60%, 70%, 80%, 90%, 95%, 99%, 99.5%, or 99.9% of the ammonia oxidizing microorganisms are inactive.

33. The method of any of the preceding claims, wherein, upon packaging, e.g., into an end use container, less than about 70%, 60%, 50%, 40%, 30%, 20%, 10%, 5%, 1%, 0.5%, or 0.1% of the ammonia oxidizing microorganisms are viable.

34. The method of any of the preceding claims, wherein, upon packaging, e.g., into an end use container, less than about 70%, 60%, 50%, 40%, 30%, 20%, 10%, 5%, 1%, 0.5%, or 0.1% of the ammonia oxidizing microorganisms are inactive.

35. The method of any of the preceding claims, wherein at least about 30%, 40%, 50%, 60%, 70%, 80%, 90%, 95%, 99%, 99.5%, 99.9%, or substantially all of the ammonia oxidizing microorganisms are heat killed, e.g., prior to packaging.

36. The method of any of the preceding claims, wherein the container, e.g., end use container, comprises a polymer bottle, e.g., a spray, aerosol, or mist bottle.

37. The method of any of the preceding claims, wherein the container, e.g., end use container, comprises a squeezable container, e.g., squeeze bottle or tube.

38. The method of any of the preceding claims, wherein the container, e.g., end use container, is substantially free of a vacuum bag.

39. The method of any of the preceding claims, wherein the container, e.g., end use container, is not configured to inhibit or reduce retrograde flow.

40. The method of any of the preceding claims, wherein the container, e.g., end use container comprises polymer, e.g., polyethylene terephthalate (PET), high density polyethylene (HDPE), polypropylene, polycarbonate, polytetrafluoroethylene (teflon®), polyviylidene fluoride (PVDF), or a cellulosic, glass, aluminum, or cardboard.

41. The method of any of the preceding claims, wherein the container, e.g., end use container, is configured to allow passage of oxygen.

42. The method of any of the preceding claims, wherein the container, e.g., end use container, is configured to allow passage of at least about 5, 10, 20, 30, 40, 50, 60, 70, 80, 90, 95, 99, or 100 percent of transmission of ionizing radiation, e.g., with gamma rays, e.g., with x-rays, e.g., from an isotope, e.g., cobalt 60, or with ultraviolet, e.g., ultraviolet C (UVC) through the container.

43. The method of any of the preceding claims, comprising treating a disease or disorder modulated by an activated immune cell in a subject.

44. The method of any of the preceding claims, wherein the activated immune cell is a T helper cell or a regulatory T cell.

45. The method of any of the preceding claims, wherein the activated immune cell is T helper type 1 (Th1), T helper type 2 (Th2), T helper type 17 (Th17), or regulatory T cell (Treg).

46. The method of any of any of the preceding claims, wherein at least about 30%, 40%, 50%, 60%, 70%, 80%, 90%, 95%, 99%, 99.5%, or 99.9% of the ammonia oxidizing microorganisms are inactive.

47. The method of any of the preceding claims, wherein administration provides for upregulation, activation, downregulation, or suppression of a cytokine associated with an activated immune cell, e.g., IL-5, IL-13, IL-4, IFNγ, IL-12, IL-2, IL-18, IL-17, IL-21, IL-22, IL-10, and TFG-β.

48. The method of any of the preceding claims, wherein the preparation is at a temperature greater than about 4° C., when administered.

49. The method of any of the preceding claims, wherein the preparation is at a temperature greater than about 10° C., when administered.

50. The method of any of the preceding claims, wherein the preparation is at a temperature greater than about room temperature e.g., between about 20° C. - 25° C., when administered.

51. The method of any of the preceding claims, wherein a period of time of at least about 1 day, 3 days, 5 days, 1 week, 2 weeks, 3 weeks, 4 weeks, 2 months, 3 months, 4 months, 5 months, 6 months, 1 year, or 5 years has elapsed between packaging and administration.

52. The method of any of the preceding claims, wherein the subject is identified as having a disease or disorder modulated by an activated immune cell.

53. The method of any of the preceding claims, wherein treatment comprises providing a therapeutic effect for a disease or disorder modulated by an activated immune cell.

54. The method of any of the preceding claims, wherein the subject is identified as having a T helper type 1 (Th1) cell mediated disease.

55. The method of any of the preceding claims, wherein the Th1 mediated disease is Celiac disease, multiple sclerosis, or diabetes, e.g., type 1 diabetes.

56. The method of any of the preceding claims, wherein treatment comprises providing a therapeutic effect for a Th1 mediated disease or a symptom thereof.

57. The method of any of the preceding claims, wherein the subject is identified as having a T helper type 2 (Th2) cell mediated disease or disorder.

58. The method of any of the preceding claims, wherein the Th2 mediated disease or disorder is associated with atopic dermatitis, allergic rhinitis, asthma, or itch.

59. The method of any of the preceding claims, wherein treatment comprises providing a therapeutic effect for a Th2 mediated disease or a symptom thereof.

60. The method of any of the preceding claims, wherein the subject is identified as having a T helper type 17 (Th17) cell mediated disease or disorder.

61. The method of any of the preceding claims, wherein the Th17 mediated disease or disorder is Autosomal dominant hyper-IgE syndrome (AD-HIES), rheumatoid arthritis, or irritable bowel syndrome.

62. The method of any of the preceding claims, wherein treatment comprises providing a therapeutic effect for a Th17 mediated disease or a symptom thereof.

63. The method of any of the preceding claims, wherein the subject is identified as having a regulatory T cell (Treg) cell mediated disease or disorder.

64. The method of any of the preceding claims, wherein the Treg mediated disease or disorder is immunodysregulation polyendocrinopathy enteropathy-X-linked (IPEX).

65. The method of any of the preceding claims, wherein treatment comprises providing a therapeutic effect for a Treg mediated disease or a symptom thereof.

66. The method of any of the preceding claims, wherein the preparation is formulated as a spray, aerosol, or mist.

67. The method of any of the preceding claims, wherein the preparation is formulated as a powder, cream, ointment, or lotion.

68. The method of any of the preceding claims, wherein the preparation comprises a thickener and/or emulsifier.

69. The method of any of the preceding claims, wherein the preparation has a viscosity of at least about 1 mPa·s, 10 mPa·s, 100 mPa·s, 1 Pa·s, 5 Pa·s, 10 Pa·s, or 20 Pa·s at room temperature, e.g., between about 20° C. - 25° C.

70. The method of any of the preceding claims, wherein the preparation comprises a talcum powder or cornstarch.

71. The method of any of the preceding claims, wherein the preparation comprises a component that is toxic to AOM, e.g., an antimicrobial or a preservative, e.g., a preservative listed in Annex VI.

72. The method of any of the preceding claims, further comprising combining the preparation with at least one preservative listed in Annex VI.

73. The method of any of the preceding claims, comprising combining the preparation with at least 500 ppb of at least one preservative listed in Annex VI.

74. The method of any of the preceding claims, wherein the preparation or formulation is administered topically.

75. The method of any of the preceding claims, wherein the preparation or formulation is administered to the body of the subject, e.g., to one or more of the face, neck, scalp, limb, hand, foot, back, buttock, torso, genitals, and chest of the subject.

76. The method of any of the preceding claims, wherein the preparation or formulation is administered intranasally.

77. The method of any of the preceding claims, comprising administering the preparation or formulation to the subject orally, enterally, intranasally, parenterally, subcutaneously, ocularly, otically, or respiratorilly.

78. The method of any of the preceding claims, wherein the preparation comprises AOM in a buffer solution, e.g., an aqueous buffer solution.

79. The method of any of the preceding claims, wherein the buffer solution, e.g., aqueous buffer solution, comprises disodium phosphate and magnesium chloride, for example, 50 mM Na₂HPO₄ and 2 mM MgCl₂ in water.

80. The method of any of the preceding claims, wherein the buffer solution e.g., aqueous buffer solution, consisting essentially of disodium phosphate and magnesium chloride, for example, 50 mM Na₂HPO₄ and 2 mM MgCl₂ in water.

81. The method of any of the preceding claims, wherein the buffer solution, e.g., aqueous buffer solution, consists of disodium phosphate and magnesium chloride, for example, 50 mM Na₂HPO₄ and 2 mM MgCl₂ in water.

82. The method of any of the preceding claims, wherein the AOM comprise ammonia oxidizing bacteria (AOB).

83. The method of any of the preceding claims, wherein the AOM consist essentially of AOB.

84. The method of any of the preceding claims, wherein the AOM consist of AOB.

85. The method of any of the preceding claims, wherein the AOM comprise Nitrosomonas, Nitrosococcus, Nitrosospira, Nitrosocystis, Nitrosolobus, Nitrosovibrio, and combinations thereof.

86. The method of any of the preceding claims, wherein the AOM is Nitrosomonas eutropha (N.eutropha).

87. The method of any of the preceding claims, wherein the AOM is N. eutropha D23, having ATCC accession number PTA-121157.

88. The method of any of the preceding claims, wherein the AOM comprise ammonia oxidizing archaea (AOA).

89. The method of any of the preceding claims, wherein administration provides for treatment of one or more of: headaches, cardiovascular diseases, inflammation, immune responses, autoimmune disorders, liver diseases, infections, neurological diseases, psychiatric disorders, pulmonary diseases, nitric oxide disorders, urea cycle disorders, congestion, vasodilation disorders, skin diseases, ophthalmic disorders, bowel disorders, auditory diseases, wound healing, reactions to insect bites, connective tissue disorders, and certain viral, bacterial, or fungal infections.

90. The method of any of the preceding claims, wherein administration provides for treatment or improvement of a local effect.

91. The method of any of the preceding claims, wherein administration provides for treatment or improvement of a systemic effect.

92. The method of any of the preceding claims, comprising:
obtaining the preparation comprising ammonia oxidizing microorganisms (AOM);
preparing a cosmetic, therapeutic, or consumer product from the preparation;
measuring at least one of AOM metabolic activity and Th1, Th2, Th17, or Treg inhibition activity of the AOM in the preparation or product to provide an activity value;
comparing the activity value to a range of pre-determined values corresponding to a pre-determined range of amounts of AOM metabolic activity and Th1, Th2, Th17, or Treg inhibition activity;
determining if the activity value is a value in the range of pre-determined values, wherein:
if the activity value is in the range of pre-determined values, classifying the preparation or product as accepted; or
if the activity value is outside the range of pre-determined values, classifying the preparation or product as not accepted.

93. The method of any of the preceding claims, further comprising heat killing a target percentage of the ammonia oxidizing microorganisms, e.g., if the preparation is not accepted.

94. A method of treating a subject, comprising:
administering to the subject a therapeutically effective amount of a preparation comprising ammonia oxidizing microorganisms (AOM),
wherein the preparation was distributed by a method of any of the preceding claims.

95. A shelf-stable preparation, comprising:
at least about 10³ cells/mL of ammonia oxidizing microorganisms (AOM); and
one or more of the following properties:
less than about 70%, 60%, 50%, 40%, 30%, 20%, 10%, 5%, 1%, 0.5%, or 0.1% of the ammonia oxidizing microorganisms are viable;
the preparation has a viscosity of at least about 1 mPa·s, 10 mPa·s, 100 mPa·s, 1 Pa·s, 5 Pa·s, 10 Pa·s, or 20 Pa·s at room temperature, e.g., between about 20° C. - 25° C.;
the preparation is formulated as a powder, cream, ointment, salve, or lotion;
the preparation comprises a component that is toxic to AOM, e.g., an antimicrobial or preservative, e.g., a preservative listed in Annex VI; and
the preparation has been sterilized.

96. A shelf-stable preparation, comprising:
at least about 10³ CFU/mL in 750-1000 mg of ammonia oxidizing microorganisms (AOM); and
at least one preservative listed in Annex VI, wherein the preparation comprises at least 500 ppb of the at least one preservative.

97. A shelf-stable preparation, comprising:
at least about 10³ CFU/mL in 750-1000 mg of ammonia oxidizing microorganisms (AOM); and
at least one preservative listed in Annex VI,
wherein the preparation if exposed to challenge with a pathogenic microorganism, will not support growth of the pathogenic microorganism.

98. The preparation of any of the preceding claims, wherein the preparation comprises at least 500 ppb of the at least one preservative listed in Annex VI.

99. The preparation of any of the preceding claims, wherein the preparation comprises a component that is toxic to AOM, e.g., an antimicrobial.

100. The preparation of any of the preceding claims, comprising at least about 10³ cells/mL, 10⁴ cells/mL, 10⁵ cells/mL, or 10⁶ cells/mL.

101. The preparation of any of the preceding claims, comprising at least about 10³ CFU/mL, 10⁴ CFU/mL, 10⁵ CFU/mL, or 10⁶ CFU/mL.

102. The preparation of any of the preceding claims, wherein the preparation has been sterilized.

103. The preparation of any of the preceding claims, wherein the preparation is substantially free of polyphosphate.

104. The preparation of any of the preceding claims, wherein, the preparation if exposed to challenge with a population of a pathogenic microorganism, will sterilize at least 30%, 40%, 50%, 60%, 70%, 80%, 90%, 95%, or 99% of the population of the microorganism.

105. The preparation of any of the preceding claims, wherein the preparation is formulated as a liquid, e.g., spray, aerosol, or mist.

106. The preparation of any of the preceding claims, wherein the preparation is formulated as a powder, cream, ointment, salve, or lotion.

107. The preparation of any of the preceding claims, wherein the preparation comprises a thickener and/or emulsifier.

108. The preparation of any of the preceding claims, wherein the preparation has a viscosity of at least about 1 mPa·s, 10 mPa·s, 100 mPa·s, 1 Pa·s, 5 Pa·s, 10 Pa·s, or 20 Pa·s at room temperature, e.g., between about 20° C. - 25° C.

109. The preparation of any of the preceding claims, wherein the preparation comprises a talcum powder or cornstarch.

110. The preparation of any of the preceding claims, packaged in an end use container.

111. The preparation of any of the preceding claims, wherein the end use container indicates one or more of the following:
storage and handling of the preparation;
formulation of the preparation;
description of contents in the preparation;
viability status of the AOM; and
directions for use of the preparation.

112. The preparation of any of the preceding claims, wherein the end use container does not indicate one or more of the following:
storage and handling of the preparation;
formulation of the preparation;
description of contents in the preparation;
viability status of the AOM; and
directions for use of the preparation.

113. The preparation of any of the preceding claims, wherein the end use container informs the subject to apply the preparation topically.

114. The preparation of any of the preceding claims, wherein the end use container informs the subject to apply the preparation intranasally.

115. The preparation of any of the preceding claims, wherein the end use container informs the subject to apply the preparation at least one of orally, enterally, intranasally, parenterally, subcutaneously, ocularly, otically, or respiratorilly.

116. The preparation of any of the preceding claims, wherein the end use container, comprises a polymer bottle, e.g., a spray, aerosol, or mist bottle.

117. The preparation of any of the preceding claims, wherein the end use container, comprises a squeezable container, e.g., squeeze bottle or tube.

118. The preparation of any of the preceding claims, wherein the end use container, is substantially free of a vacuum bag.

119. The preparation of any of the preceding claims, wherein the end use container, is not configured to inhibit or reduce retrograde flow.

120. The preparation of any of the preceding claims, wherein the end use container comprises polymer, e.g., polyethylene terephthalate (PET), high density polyethylene (HDPE), polypropylene, polycarbonate, polytetrafluoroethylene (teflon®), polyviylidene fluoride (PVDF), or a cellulosic, glass, aluminum, or cardboard.

121. The preparation of any of the preceding claims, wherein the end use container, is configured to allow passage of oxygen.

122. The preparation of any of the preceding claims, wherein the end use container, is configured to allow passage of at least about 5, 10, 20, 30, 40, 50, 60, 70, 80, 90, 95, 99, or 100 percent of transmission of ionizing radiation, e.g., with gamma rays, e.g., with x-rays, e.g., from an isotope, e.g., cobalt 60, or with ultraviolet, e.g., ultraviolet C (UVC) through the container.

123. The preparation of any of the preceding claims, wherein the preparation is formulated for oral, enteral (e.g., buccal, sublingual, sublabial, and rectal), parenteral (e.g., subcutaneous, intradermal, intramuscular, intravenous, and intraarticular), inhalation (e.g., fine particle dusts or mists which may be generated by means of various types of metered doses, pressurized aerosols, nebulizers or insufflators, and including intranasally or via the lungs), intranasal, eye, ear, rectal, injection, urogenital, or topical (e.g., dermal, transdermal, transmucosal, buccal, sublingual, and intraocular) administration.

124. The preparation of any of the preceding claims, wherein the preparation is formulated for treatment of one or more of: headaches, cardiovascular diseases, inflammation, immune responses, autoimmune disorders, liver diseases, infections, neurological diseases, psychiatric disorders, nitric oxide disorders, urea cycle disorders, congestion, vasodilation disorders, skin diseases, ophthalmic disorders, wound healing, reactions to insect bites, connective tissue disorders, and certain viral, bacterial, or fungal infections.

125. The preparation of any of the preceding claims, wherein the preparation is formulated for treatment of a disease or disorder modulated by an activated immune cell.

126. The preparation of any of the preceding claims, wherein the activated immune cell is a T helper cell or regulatory T cell, e.g., T helper type 1 (Th1), T helper type 2 (Th2), T helper type 17 (Th17), or regulatory T cell (Treg).

127. The preparation of any of the preceding claims, wherein the preparation is formulated for upregulation, activation, downregulation, or suppression of a cytokine associated with an activated immune cell, e.g., IL-5, IL-13, IL-4, IFNγ, IL-12, IL-2, IL-18, IL-17, IL-21, IL-22, IL-10, and TFG-B.

128. The preparation of any of the preceding claims, wherein the preparation is formulated for treatment of a Th1 mediated disease or disorder, e.g., Celiac disease, multiple sclerosis, or diabetes, e.g., type 1 diabetes.

129. The preparation of any of the preceding claims, wherein the preparation is formulated for treatment of a Th2 mediated disease or disorder, e.g., atopic dermatitis, allergic rhinitis, asthma, or itch.

130. The preparation of any of the preceding claims, wherein the preparation is formulated for treatment of a Th17 mediated disease or disorder, e.g., Job’s syndrome, rheumatoid arthritis, irritable bowel syndrome.

131. The preparation of any of the preceding claims, wherein the preparation is formulated for treatment of a Treg mediated disease or disorder, e.g., immunodysregulation polyendocrinopathy enteropathy-X-linked (IPEX).

132. The preparation of any of the preceding claims, wherein the preparation comprises AOM in a buffer solution, e.g., an aqueous buffer solution.

133. The preparation of any of the preceding claims, wherein the buffer solution, e.g., aqueous buffer solution, comprises disodium phosphate and magnesium chloride, for example, 50 mM Na₂HPO₄ and 2 mM MgCl₂ in water.

134. The preparation of any of the preceding claims, wherein the buffer solution e.g., aqueous buffer solution, consisting essentially of disodium phosphate and magnesium chloride, for example, 50 mM Na₂HPO₄ and 2 mM MgCl₂ in water.

135. The preparation of any of the preceding claims, wherein the buffer solution, e.g., aqueous buffer solution, consists of disodium phosphate and magnesium chloride, for example, 50 mM Na₂HPO₄ and 2 mM MgCl₂ in water.

136. The preparation of any of the preceding claims, wherein the AOM comprise ammonia oxidizing bacteria (AOB).

137. The preparation of any of the preceding claims, wherein the AOM consist essentially of AOB.

138. The preparation of any of the preceding claims, wherein the AOM consist of AOB.

139. The preparation of any of the preceding claims, wherein the AOM comprise Nitrosomonas, Nitrosococcus, Nitrosospira, Nitrosocystis, Nitrosolobus, Nitrosovibrio, and combinations thereof.

140. The preparation of any of the preceding claims, wherein the AOM is Nitrosomonas eutropha (N. eutropha).

141. The preparation of any of the preceding claims, wherein the AOM is N. eutropha D23, having ATCC accession number PTA-121157.

142. The preparation of any of the preceding claims, wherein less than about 70%, 60%, 50%, 40%, 30%, 20%, 10%, 5%, 1%, 0.5%, or 0.1% of the ammonia oxidizing microorganisms are viable.

143. The preparation of any of the preceding claims, wherein at least about 30%, 40%, 50%, 60%, 70%, 80%, 90%, 95%, 99%, 99.5%, or 99.9% of the ammonia oxidizing microorganisms are inactive.

144. A method of treating a subject, comprising:
administering to the subject a therapeutically effective amount of the preparation of any of the preceding claims,
thereby treating the subject.
